(19) United States
(12) Patent Application Publication
(10) Pub. No.: US 2007/0027145 A1
(43) Pub. Date: Feb. 1, 2007

(54) Quinazoline derivatives as inhibitors of vegf receptor tyrosine kinases

(76) Inventor: Laurent Francois Andre Hennequin, Reims (FR)

Correspondence Address: MORGAN LEWIS & BOCKIUS LLP, 1111 PENNSYLVANIA AVENUE NW, WASHINGTON, DC, 20004, US

(21) Appl. No.: 10/566,841

(22) PCT Filed: Aug. 5, 2004
(86) PCT No.: PCT/GB04/03393

§ 371 Date: Feb. 2, 2006

(30) Foreign Application Priority Data
Aug. 6, 2003 (GB) 0318423.1

(51) Int. Cl. A61K 31/541; A61K 31/5377; A61K 31/517; C07D 417/02; C07D 413/02; C07D 403/02

(52) U.S. Cl. 514/227.8; 514/234.2; 514/252.16; 514/266.4; 514/266.3; 514/266.2; 544/60; 544/120; 544/284; 544/287; 544/293

(57) ABSTRACT

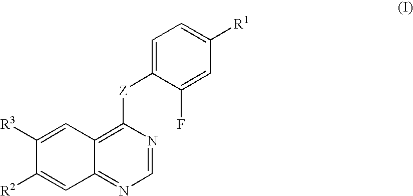

The present invention relates to compounds of the Formula (I): wherein Z is —NH—, —O— or —S—; R¹ represents bromo or chloro; R³ represents C₁₋₃alkoxy or hydrogen; R² is selected from one of the following three groups: (i) Q¹X¹- wherein X¹ and Q¹ are as defined herein; (ii) Q¹⁵W³— wherein Q¹⁵ and W³ are as defined herein; and (iii) Q²¹W⁴C₁₋₅alkylX¹ wherein X¹, W⁴ and Q²¹ are as defined herein; and salts thereof; their use in the manufacture of a medicament for use in the production of an antiangiogenic and/or vascular permeability reducing effect in warm blooded animals; processes for the preparation of such compounds; pharmaceutical compositions containing a compound of formula (I) or a pharmaceutically acceptable salt thereof and methods of treating disease states involving angiogenesis by administering a compound of formula (I) or a pharmaceutically acceptable salt thereof. The compounds of formula (I) inhibit the effects of VEGF, a property of value in the treatment of a number of disease states including cancer and rheumatoid arthritis.

The present invention relates to quinazoline derivatives, processes for their preparation, pharmaceutical compositions containing them as active ingredient, methods for the treatment of disease states associated with angiogenesis and/or increased vascular permeability, to their use as medicaments and to their use in the manufacture of medicaments for use in the production of antiangiogenic and/or vascular permeability reducing effects in warm-blooded animals such as humans.

Normal angiogenesis plays an important role, in a variety of processes including embryonic development, wound healing and several components of female reproductive function. Undesirable or pathological angiogenesis has been associated with disease states including diabetic retinopathy, psoriasis, cancer, rheumatoid arthritis, atheroma, Kaposi's sarcoma and haemangioma (Fan et al, 1995, Trends Pharmacol. Sci. 16: 57-66; Folkman, 1995, Nature Medicine 1: 27-31). Alteration of vascular permeability is thought to play a role in both normal and pathological physiological processes (Cullinan-Bove et al, 1993, Endocrinology 133: 829-837; Senger et al, 1993, Cancer and Metastasis Reviews, 12: 303-324). Several polypeptides with in vitro endothelial cell growth promoting activity have been identified including, acidic and basic fibroblast growth factors (aFGF & bFGF) and vascular endothelial growth factor (VEGF). By virtue of the restricted expression of its receptors, the growth factor activity of VEGF, in contrast to that of the FGFs, is relatively specific towards endothelial cells. Recent evidence indicates that VEGF is an important stimulator of both normal and pathological angiogenesis (Jakeman et al, 1993, Endocrinology, 133: 848-859; Kolch et al, 1995, Breast Cancer Research and Treatment, 36:139-155) and vascular permeability (Connolly et al, 1989, J. Biol. Chem. 264: 20017-20024). Antagonism of VEGF action by sequestration of VEGF with antibody can result in inhibition of tumour growth (Kim et al, 1993, Nature 362: 841-844). Basic FGF (bFGF) is a potent stimulator of angiogenesis (e.g. Hayek et al, 1987, Biochem. Biophys. Res. Commun. 147: 876-880) and raised levels of FGFs have been found in the serum (Fujimoto et al, 1991, Biochem. Biophys. Res. Commun. 180: 386-392) and urine (Nguyen et al, 1993, J. Natl. Cancer. Inst. 85: 241-242) of patients with cancer.

Receptor tyrosine kinases (RTKs) are important in the transmission of biochemical signals across the plasma membrane of cells. These transmembrane molecules characteristically consist of an extracellular ligand-binding domain connected through a segment in the plasma membrane to an intracellular tyrosine kinase domain. Binding of ligand to the receptor results in stimulation of the receptor-associated tyrosine kinase activity which leads to phosphorylation of tyrosine residues on both the receptor and other intracellular molecules. These changes in tyrosine phosphorylation initiate a signalling cascade leading to a variety of cellular responses. To date, at least nineteen distinct RTK subfamilies, defined by amino acid sequence homology, have been identified. One of these subfamilies is presently comprised by the fms-like tyrosine kinase receptor, Flt-1, the kinase insert domain-containing receptor, KDR (also referred to as Flk-1), and another fms-like tyrosine kinase receptor, Flt-4. Two of these related RTKS, Flt-1 and KDR, have been shown to bind VEGF with high affinity (De Vries et al, 1992, Science 255: 989-991; Terman et al, 1992, Biochem. Biophys. Res. Comm. 1992, 187: 1579-1586). Binding of VEGF to these receptors expressed in heterologous cells has been associated with changes in the tyrosine phosphorylation status of cellular proteins and calcium fluxes.

The present invention is based on the discovery of compounds that inhibit the effects of VEGF, a property of value in the treatment of disease states associated with angiogenesis and/or increased vascular permeability such as cancer, diabetes, psoriasis, rheumatoid arthritis, Kaposi's sarcoma, haemangioma, lymphoedema, acute and chronic nephropathies, atheroma, arterial restenosis, autoimmune diseases, acute inflammation, excessive scar formation and adhesions, endometriosis, dysfunctional uterine bleeding and ocular diseases with retinal vessel proliferation including macular degeneration.

VEGF is a key stimulus for vasculogenesis and angiogenesis. This cytokine induces a vascular sprouting phenotype by inducing endothelial cell proliferation, protease expression and migration, and subsequent organisation of cells to form a capillary tube (Keck, P. J., Hauser, S. D., Krivi, G., Sanzo, K., Warren, T., Feder, J., and Connolly, D. T., Science (Washington DC), 246: 1309-1312, 1989; Lamoreaux, W. J., Fitzgerald, M. E., Reiner, A., Hasty, K. A., and Charles, S. T., Microvasc. Res., 55: 29-42, 1998; Pepper, M. S., Montesano, R., Mandroita, S. J,, Orci, L. and Vassalli J. D., Enzyme Protein, 49: 138-162, 1996.). In addition, VEGF induces significant vascular permeability (Dvorak, H. F., Detmar, M., Claffey, K. P., Nagy, J. A., van de Water, L., and Senger, D. R., (Int. Arch. Allergy Immunol., 107: 233-235, 1995; Bates, D. O., Heald, R. I., Curry, F. E. and Williams, B. J. Physiol. (Lond.), 533: 263-272, 2001), promoting formation of a hyper-permeable, immature vascular network which is characteristic of pathological angiogenesis.

It has been shown that activation of KDR alone is sufficient to promote all of the major phenotypic responses to VEGF, including endothelial cell proliferation, migration, and survival, and the induction of vascular permeability (Meyer, M., Clauss, M., Lepple-Wienhues, A., Waltenberger, J., Augustin, H. G., Ziche, M., Lanz, C., Büttner, M., Rziha, H-J., and Dehio, C., EMBO J., 18: 363-374, 1999; Zeng, H., Sanyal, S. and Mukhopadhyay, D., J. Biol. Chem., 276: 32714-32719, 2001; Gille, H., Kowalski, J., Li, B., LeCouter, J., Moffat, B, Zioncheck, T. F., Pelletier, N. and Ferrara, N., J. Biol. Chem., 276: 3222-3230, 2001).

International patent applications publication numbers WO 98/13354, WO 01/32651 and WO 01/77085 describe VEGF receptor tyrosine kinase inhibitors. International patent application publication number WO 01 /21594 describes a broad scope of quinazoline derivatives but with a different activity to those of the present invention; compounds of WO 01/21594 inhibit aurora-2 kinase. Compounds of WO 98/13354 and WO 01/32651 possess activity against VEGF receptor tyrosine kinase (RTK) and also possess some activity against epidermal growth factor (EGF) RTK. International patent application publication number WO 02/18372 and European Patent Application No. EP0566226 describe anilinoquinazolines which inhibit EGF RTK. International patent applications publication numbers WO 00/55141 and WO 04/006846 also describe inhibitors of EGF RTK. The compounds of WO 98/13354 and WO 01/32651 are generally more potent against KDR than against Flt-1 and generally they are more potent against VEGF RTK than against EGF RTK. A potential problem with some VEGF RTK inhibitors is that they have been found to act as potassium channel blockers and are positive in a hERG assay; such activity may give rise to ECG (electrocardiogram) changes in vivo.

Surprisingly we have now found compounds of the present invention to be potent KDR and/or Flt-1 inhibitors as well as potent inhibitors of EGF RTK and to be inactive or only weakly active in a hERG assay.

According to one aspect of the present invention there is provided a compound of the formula I: 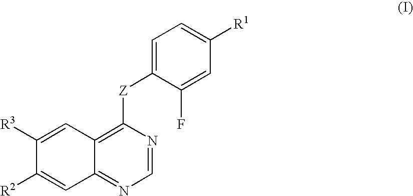
wherein:
Z is —NH—, —O— or —S—;
R¹ represents bromo or chloro;
R³ represents C₁₋₃alkoxy or hydrogen;
R² is selected from one of the following three groups:
(i) Q¹X¹-
wherein X¹ represents —O—, —S— or —NR⁴— wherein R⁴ is hydrogen, C₁₋₃alkyl or C₁₋₃alkoxyC₂₋₃alkyl and Q¹ is selected from one of the following ten groups:
1) Q² (wherein Q² is a 5-6-membered saturated or partially unsaturated heterocyclic group with 1-2 heteroatoms, selected independently from O, S and N, which heterocyclic group bears at least one substituent selected from C₂₋₅alkenyl, C₂₋₅alkyl C₁₋₆fluoroalkyl, aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, C₁₋₆fluoroalkanoyl, carbamoylC₁₋₆alkyl, C₁₋₄alkylcarbamoylC₁₋₆alkyl, di(C₁₋₄alkyl)carbamoylC₁₋₆alkyl, C₁₋₆alkylsulphonyl and C₁₋₆fluoroalkylsulphonyl and which heterocyclic group may optionally bear a further 1 or 2 substituents selected from C₂₋₅alkenyl, C₂₋₅alkynyl, C₁₋₆fluoroalkyl, C₁₋₆alkanoyl, aminoC₂₋₆alkanoyl, C₁₋₄alkylaminoC₂₋₆alkanoyl di(C₁₋₄alkyl)aminoC₂₋₆alkanoyl, C₁₋₄alkoxyC₁₋₄alkylaminoC₂₋₆alkanoyl, C₁₋₆fluoroalkanoyl, carbamoyl, C₁₋₄alkylcarbamoyl, di(C₁₋₄alkylcarbamoyl, carbamoylC₁₋₆alkyl, C₁₋₄alkylcarbarmoylC₁₋₆alkyl, di(C₁₋₄alkyl)carbamoylC₁₋₆alkyl, C₁₋₆alkylsulphonyl, C₁₋₆fluoroalkylsulphonyl, oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄alkyl, C₁₋₄alkylsulphonylC₁₋₄alkyl, C₁₋₄alkoxycarbonyl, C₁₋₄aminoalkyl, C₁₋₄alkylamino, di(C₁₋₄alkyl)amino, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g} ring D (wherein f is 0 or 1, g is 0 or 1 and ring D is a 5-6-membered saturated or partially unsaturated heterocyclic group with 1-2 heteroatoms, selected independently from O, S and N, which cyclic group may bear one or more substituents selected from C₁₋₄alkyl),
or Q² bears a single substituent selected from methylenedioxy and ethylenedioxy);
with the proviso that if Q¹ is Q² and X¹ is —O— then Q² must bear at least one substituent selected from C₂₋₅alkenyl, C₂₋₅alkynyl, C₁₋₄alkoxyC₁₋₄alkylaminoC₂₋₆alkanoyl, carbamoylC₁₋₆alkyl, C₁₋₄alkylcarbamoylC₁₋₆alkyl, and di(C₁₋₄alkyl)carbamoylC₁₋₆alkyl and optionally may bear a further 1 or 2 substituents as defined hereinbefore;
2) C₁₋₅alkylW¹Q² (wherein W¹ represents —O—, —S—, —SO—, —SO₂—, —C(O)—, —OC(O)—, —NQ³C(O)—, —C(O)NQ⁴-, —SO₂NQ⁵-, —NQ⁶SO₂— or —NQ⁷- (wherein Q³, Q⁴, Q⁵, Q⁶ and Q⁷ each independently represents hydrogen, C₁₋₃alkyl, C₁₋₃alkoxyC₂₋₃alkyl, C₂₋₅alkenyl, C₂₋₅alkenyl, or C₁₋₄haloalkyl) and Q² is as defined hereinbefore;
3) C₁₋₅alkylQ² (wherein Q² is as defined hereinbefore);
4) C₂₋₅alkenylQ² (wherein Q² is as defined hereinbefore);
5) C₂₋₅alkynylQ² (wherein Q² is as defined hereinbefore);
6) C₁₋₄alkylW²C₁₋₄alkylQ² (wherein W² represents —O—, —S—, —SO—, —SO₂—, —C(O)—, —OC(O)—NQ⁸C(O)—, —C(O)NQ⁹-, —SO₂NQ¹⁰-, —NQ¹¹SO— or —NQ¹²- (wherein Q⁸, Q⁹, Q¹⁰, Q¹¹ and Q¹² each independently represents hydrogen, C₁₋₃alkyl, C₁₋₃alkoxyC₂₋₃alkyl, C₂₋₅alkenyl, C₂₋₅alkynyl or C₁₋₄haloalkyl) and Q² is as defined hereinbefore);
7) C₂₋₅alkenylW²C₁₋₄alkylQ² (wherein W² and Q² are as defined hereinbefore);
8) C₂₋₅alkynylW²C₁₋₄alkylQ² (wherein W² and Q² are as defined hereinbefore);
9) C₁₋₄alkylQ¹³(C₁₋₄alkyl)ⱼ(W²)ₖQ¹⁴ (wherein W² is as defined hereinbefore, j is 0 or 1, k is 0 or 1, and Q¹³ and Q¹⁴ are each independently selected from hydrogen, C₁₋₃alkyl, cyclopentyl, cyclohexyl and a 5-6-membered saturated or partially unsaturated heterocyclic group- with 1-2 heteroatoms, selected independently from O, S and N, which C₁₋₃alkyl group may bear 1 or 2 substituents selected from oxo, hydroxy, halogeno and C₁₋₄alkoxy and which cyclic group may bear 1, 2 or 3 substituents selected from C₂₋alkenyl, C₂₋₅alkynyl, C₁₋₆fluoroalkyl, C₁₋₆alkanoyl, aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, C₁₋₆fluoroalkanoyl, carbamoyl, C₁₋₄alkylcarbamoyl, di(C₁₋₄alkyl)carbamoyl, carbamoylC₁₋₆alkyl, C₁₋₄alkylcarbamoylC₁₋₆alkyl, di(C₁₋₄alkyl)carbamoylC₁₋₆alkyl, C₁₋₆alkylsulphonyl, C₁₋₆fluoroalkylsulphonyl, oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄alkyl, C₁₋₄alkylsulphonylC₁₋₄alkyl, C₁₋₄alkoxycarbonyl, C₁₋₄aminoalkyl, C₁₋₄alkylamino, di(C₁₋₄alkyl)amino, C₁₋₄alkylaminoC₁₋₄alkyl, di(C₁₋₄alkyl)aminoC₁₋₄alkyl C₁₋₄alkylaminoC₁₋₄alkoxy, di(C₁₋₄alkyl)aminoC₁₋₄alkoxy and a group —(—O—)_{f}(C₁₋₄alkyl)_{g} ring D (wherein f is 0 or 1, g is 0 or 1 and ring D is a 5-6-membered saturated or partially unsaturated heterocyclic group with 1-2 heteroatoms, selected independently from O, S and N, which heterocyclic group may bear one or more substituents selected from C₁₋₄alkyl), with the provisos that Q¹³ cannot be hydrogen and one or both of Q¹³ and Q¹⁴ must be a 5-6-membered saturated or partially unsaturated heterocyclic group as defined hereinbefore which heterocyclic group bears at least one substituent selected from C₂₋₅alkenyl, C₂₋₅alkynyl, C₁₋₄fluoroalkyl, C₁₋₆alkanoyl, aminoC₂₋₆alkanoyl, C₁₋₄-alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, C₁₋₆fluoroalkanoyl, carbamoyl, C₁₋₄alkylcarbamoyl, di(C₁₋₄alkyl)carbamoyl, carbamoylC₁₋₆alkyl, C₁₋₄-alkylcarbamoylC₁₋₆alkyl, di(C₁₋₄alkyl)carbamoylC₁₋₆alkyl, C₁₋₆alkylsulphonyl and C₁₋₆fluoroalkylsulphonyl and which heterocyclic group optionally bears 1 or 2 further substituents selected from those defined hereinbefore); and
10) C₁₋₄alkylQ¹³-C(O)-C₁₋₄alkylQ¹⁴ⁿ wherein Q¹³ is as defined hereinbefore and is not hydrogen and Q¹⁴ⁿ is a 5-6membered saturated or partially unsaturated heterocyclic group containing at least one nitrogen atom and optionally containing a further heteroatom selected from N and O wherein Q¹⁴ⁿ is linked to C₁₋₆alkyl via a nitrogen atom or a carbon atom and wherein Q¹⁴ⁿ optionally bears 1, 2 or 3 substituents selected from C₂₋₅alkenyl, C₂₋₅alkynyl, C₁₋₆fluoroalkyl, C₁₋₆alkanoyl, aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₁₋₄alkanoyl, C₁₋₆fluoroalkanoyl, carbamoyl, C₁₋₄alkylcarbamoyl, di(C₁₋₄alkyl)carbamoyl, carbamoylC₁₋₆alkyl, C₁₋₄alkylcarbamoylC₁₋₆alkyl, di(C₁₋₄alkyl)carbamoylC₁₋₄alkyl, C₁₋₆alkylsulphonyl, C₁₋₆fluoroalkylsulphonyl, oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄alkyl, C₁₋₄alkylsulphonylC₁₋₄alkyl, C₁₋₄alkoxycarbonyl, C₁₋₄aminoalkyl, C₁₋₄alkylamino, di(C₁₋₄alkyl)amino, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g} ring D (wherein f is 0 or 1, g is 0 or 1 and ring D is a 5-6-membered saturated or partially unsaturated heterocyclic group with 1-2 heteroatoms, selected independently from O, S and N, which heterocyclic group may bear one or more substituents selected from C₁₋₄alkyl)
or Q¹⁴ⁿ bears a single substituent selected from methylenedioxy and ethylenedioxy);
(ii) Q¹⁵W³—
wherein W³ represents —NQ¹⁶C(O)—, —C(O)NQ¹⁷-, —SO₁NQ¹⁸-, —NQ¹⁹SO₂— or —NQ²⁰- (wherein Q¹⁶, Q¹⁷, Q¹⁸, Q¹⁹ and Q²⁰ each independently represents C₂₋₅alkenyl, C₂₋₅alkynyl, C₁₋₄haloalkyl), and Q¹⁵ is C₁₋₆haloalkyl, C₂₋₅alkenyl or C₂₋₅alkynyl; and
(iii) Q²¹W⁴C₁₋₅alkylX¹ wherein X¹ is as defined hereinbefore, W⁴ represents —NQ²²C(O)—, —C(O)NQ²³-, —SO₂NQ²⁴-, —NQ SO₂— or —NQ²⁶ (wherein Q²², Q²³, Q²⁴, Q²⁵ and Q²⁶ each independently represents hydrogen, C₁₋₃alkyl, C₁₋₃alkoxyC₂₋₃alkyl, C₂₋₅alkenyl, C₂₋₅alkynyl or C₁₋₄haloalkyl), and Q²¹ represents C₁₋₆haloalkyl, C₂₋₅alkenyl or C₂₋₅alkynyl;
   or a salt thereof or a prodrug thereof.

According to one aspect of the present invention Z is —NH—.

According to one aspect of the present invention R³ is methoxy.

According to one aspect of the present invention X¹ is —O—;

According to one aspect of the present invention R² is selected from group (i) of the groups (i), (ii) and (iii) defined hereinbefore.

According to one aspect of the present invention R² is selected from group (ii) of the groups (i), (ii) and (iii) defined hereinbefore.

According to one aspect of the present invention R² is selected from group (iii) of the groups (i), (ii) and (iii) defined hereinbefore.

According to one aspect of the present invention R² is selected from:
Q¹X¹-
wherein X¹ is as defined hereinbefore and Q¹ is selected from one of the following ten groups:
1) Q² (wherein Q² is a 5-6-membered saturated or partially unsaturated heterocyclic group with 1-2 heteroatoms, selected independently from O, S and N, which heterocyclic group bears at least one substituent selected from C₂₋₅alkenyl, C₂₋₅alkynyl, C₁₋₆fluoroalkyl, aminoC₂₋₆alkanoyl, C₁₋₄alkylaminoC₂₋₆alkanoyl, -di(C₁₋₄alkyl)aminoC₂₋₆alkanoyl, C₁₋₄alkoxyC₁₋₄alkylaminoC₂₋₆alkanoyl, C₁₋₆-fluoroalkanoyl, carbamoylC₁₋₆alkyl, C₁₋₄alkylcarbamoylC₁₋₆alkyl, di(C₁₋₄alkyl)carbamoylC₁₋₆alkyl, C₁₋₆alkylsulphonyl and C₁₋₄fluoroalkylsulphonyl and which heterocyclic group may optionally bear a further 1 or 2 substituents selected from C₂₋₅alkenyl, C₂₋₅alkynyl, C₁₋₆fluoroalkyl, C₁₋₆alkanoyl, aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, C₁₋₆fluoroalkanoyl, carbamoyl, C₁₋₄alkylcarbamoyl, di(C₁₋₄alkyl)carbamoyl, carbamoylC₁₋₆alkyl, C₁₋₄alkylcarbamoylC₁₋₆alkyl, di(C₁₋₄alkyl)carbamoylC₁₋₆alkyl, C₁₋₆alkylsulphonyl, C₁₋₆fluoroalkylsulphonyl, oxo, hydroxy, halogeno, cyano, C₁₋₄cyanoalkyl, C,₄alkyl, C₁₋₄hydroxyalkyl, C₁₋₄alkoxy, C₁₋₄alkoxyC₁₋₄alkyl, C₁₋₄alkylsulphonylC₁₋₄alkyl, C₁₋₄alkoxycarbonyl, C₁₋₄aminoalkyl, C₁₋₄alkylamino, di(C₁₋₄alkyl)amino, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g} ring D (wherein f is 0 or 1, g is 0 or 1 and ring D is a 5-6-membered saturated or partially unsaturated heterocyclic group with 1-2 heteroatoms, selected independently from O, S and N, which cyclic group may bear one or more substituents selected from C₁₋₄alkyl),
or Q² bears a single substituent selected from methylenedioxy and ethylenedioxy);
with the proviso that if Q¹ is Q² and X¹ is —O— then Q² must bear at least one substituent selected from C₂₋₅alkenyl, C₂₋₅alkynyl, C₁₋₄alkoxyC₁₋₄alkylaminoC₂₋₆alkanoyl, carbamoylC₁₋₆alkyl, C₁₋₄alkylcarbamoylC₁₋₆alkyl, and di(C₁₋₄alkyl)carbamoylC₁₋₆alkyl and optionally may bear a further 1 or 2 substituents as defined hereinbefore;
2) C₁₋₅alkylW¹Q² (wherein W¹ represents —O—, —S—, —SO—, —SO₂—, —C(O)—, —OC(O)—, —NQ³C(O)—, —C(O)NQ⁴-, —SO₂NQ⁵-, —NQ⁶SO₂— or —NQ⁷- (wherein Q³, Q⁴, Q⁵, Q⁶ and Q⁷ each independently represents hydrogen, C₁₋₃alkyl, C₁₋₃alkoxyC₂₋₃alkyl, C₂₋₅alkenyl, C₂₋₅alkyl or C₁₋₄haloalkyl) and Q² is as defined hereinbefore;
3) C₁₋₅alkylQ² (wherein Q² is as defined hereinbefore);
4) C₂₋₅alkenylQ² (wherein Q² is as defined hereinbefore);
5) C₂₋₅alkynylQ² (wherein Q² is as defined hereinbefore);
6) C₁₋₄alkylW²C₁₋₄alkylQ² (wherein W² represents —O—, —S—, —SO—, —SO₂—, —C(O)—, —OC(O)—NQ⁸C(O)—, —C(O)NQ⁹-, —SO₂NQ¹⁰-, —NQ¹¹SO₂— or —NQ¹²- (wherein Q⁸, Q⁹, Q¹⁰, Q¹¹ and Q¹² each independently represents hydrogen, C₁₋₃alkyl, C₁₋₃alkoxyC₂₋₃alkyl, C₂₋₅alkenyl, C₂₋₅alkynyl or C₁₋₄haloalkyl) and Q² is as defined hereinbefore);
7) C₂₋₅alkenylW²C₁₋₄alkylQ² (wherein W² and Q² are as defined hereinbefore);
8) C₂₋₅alkynylW²C₁₋₄alkylQ W² Q are as defined hereinbefore);
9) C₁₋₄alkylQ¹³(C₁₋₄alkyl)ⱼ(W²)ₖQ¹⁴ (wherein W² is as defined hereinbefore, j is 0 or 1, k is 0 or 1, and Q¹³ and Q¹⁴ are each independently selected from hydrogen, C₁₋₃alkyl, cyclopentyl, cyclohexyl and a 5-6-membered saturated or partially unsaturated heterocyclic group with 1-2 heteroatoms, selected independently from O, S and N, which C₁₋₃alkyl group may bear 1 or 2 substituents selected from oxo, hydroxy, halogeno and C₁₋₄alkoxy and which cyclic group may bear 1, 2 or 3 substituents selected from C₂₋₅alkenyl, C₂₋₅alkynyl, C₁₋₆fluoroalkyl, C₁₋₆alkanoyl, aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, C₁₋₆fluoroalkanoyl, carbamoyl, C₁₋₄alkylcarbamoyl, di(C₁₋₄alkyl)carbamoyl, carbamoylC₁₋₆alkyl, C₁₋₄alkylcarbamoylC₁₋₆alkyl, di(C₁₋₄alkyl)carbamoylC₁₋₆alkyl, C₁₋₆alkylsulphonyl, C₁₋₆fluoroalkylsulphonyl, oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄alkyl, C₁₋₄alkylsulphonylC₁₋₄alkyl, C₁₋₄alkoxycarbonyl, C₁₋₄aminoalkyl, C₁₋₄alkylamino, di(C₁₋₄alkyl)amino, C₁₋₄alkylaminoC₁₋₄alkyl, di(C₁₋₄kyl)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g} ring D (wherein f is 0 or 1, g is 0 or 1 and ring D is a 5-6-membered saturated or partially unsaturated heterocyclic group with 1-2 heteroatoms, selected independently from O, S and N, which heterocyclic group may bear one or more substituents selected from C₁₋₄alkyl), with the provisos at Q¹³ cannot be hydrogen and one or both of Q¹³ and Q¹⁴ must be a 5-6-membered saturated or partially unsaturated heterocyclic group as defined hereinbefore which heterocyclic group bears at least one substituent selected from C₂₋₅alkenyl, C₂₋₅alkynyl, C₁₋₆fluoroalkyl, C₁₋₆alkanoyl, aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, C₁₋₆fluoroalkanoyl, carbamoyl, C₁₋₄alkylcarbamoyl, di(C₁₋₄alkyl)carbamoyl, carbamoylC₁₋₆alkyl, C₁₋₄alkylcarbamoylC₁₋₆alkyl, di(C₁₋₄alkyl)carbamoylC₁₋₆alkyl, C₁₋₆alkylsulphonyl and C₁₋₆fluoroalkylsulphonyl and which heterocyclic group optionally bears 1 or 2 further substituents selected from those defined hereinbefore); and
10) C₁₋₄alkylQ¹³-C(O)-C₁₋₄alkylQ¹⁴ⁿ wherein Q¹³ is as defined hereinbefore and is not hydrogen and Q¹⁴ⁿ is a 5-6-membered saturated or partially unsaturated heterocyclic group containing at least one nitrogen atom and optionally containing a further heteroatom selected from N and O wherein Q¹⁴ⁿ is linked to C₁₋₆alkyl via a nitrogen atom and wherein Q¹⁴ⁿ optionally bears 1, 2 or 3 substituents selected from C₂₋₅-alkenyl, C₂₋₅alkynyl, C₁₋₆fluoroalkyl, C₁₋₆alkanoyl, aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, C₁₋₆fluoroalkanoyl, carbamoyl, C₁₋₄alkylcarbamoyl, di(C₄₁₋alkyl)carbamoyl, carbamoylC₁₋₆alkyl, C₁₋₄alkylcarbamoylC₁₋₆alkyl, di(C₁₋₄alkyl)carbamoylC₁₋₆alkyl, C₁₋₆alkylsulphonyl, C₁₋₆fluoroalkylsulphonyl, oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄alkyl, C₁₋₄alkylsulphonylC₁₋₄alkyl, C₁₋₄alkoxycarbonyl, C₁₋₄aminoalkyl, C₁₋₄alkylamino, di(C₁₋₄alkyl)amino, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g} ring D (wherein f is 0 or 1, g is 0 or 1 and ring D is a 5-6-membered saturated or partially unsaturated heterocyclic group with 1-2 heteroatoms, selected independently from O, S and N, which heterocyclic group may bear one or more substituents selected from C₁₋₄alkyl)
or Q¹⁴ⁿ bears a single substituent selected from methylenedioxy and ethylenedioxy).
According to one aspect of the present invention R² is selected from:
Q¹X¹-
wherein X¹ is as defined hereinbefore and Q¹ is selected from one of the following ten groups:
1) Q² (wherein Q² is a 5-6-membered saturated or partially unsaturated heterocyclic group with 1-2 heteroatoms, selected independently from O, S and N, which heterocyclic group bears at least one substituent selected from C₂₋₅alkenyl, C₂₋₅alkynyl, aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, C₁₋₆fluoroalkanoyl, carbamoylC₁₋₆alkyl, C₁₋₄alkylcarbamoylC₁₋₆alkyl, di(C₁₋₄alkyl)carbamoylC₁₋₆alkyl, C₁₋₄alkylsulphonyl and C₁₋₆fluoroalkylsulphonyl and which heterocyclic group may optionally bear a further 1 or 2 substituents selected from C₂₋₅alkenyl, C₂₋₅alkynyl, C₁₋₆fluoroalkyl, C₁₋₆alkanoyl, aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, C₁₋₆fluoroalkanoyl, carbamoyl, C₁₋₄alkylcarbamoyl, di(C₁₋₄alkyl)carbamoyl, carbamoylC₁₋₆alkyl, C₁₋₄alkylcarbamoylC₁₋₆alkyl, di(C₁₋₄alkyl)carbamoylC₁₋₆alkyl, C₁₋₆alkylsulphonyl, C₁₋₆fluoroalkylsulphonyl, oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄alkyl, C₁₋₄alkylsulphonylC₁₋₄alkyl, C₁₋₄alkoxycarbonyl, C₁₋₄aminoalkyl, C₁₋₄alkylamino, di(C₁₋₄alkyl)amino, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g} ring D (wherein f is 0 or 1, g is 0 or 1 and ring D is a 5-6-membered saturated or partially unsaturated heterocyclic group with 1-2 heteroatoms, selected independently from O, S and N, which cyclic group may bear one or more substituents selected from C₁₋₄alkyl),
or Q² bears a single substituent selected from methylenedioxy and ethylenedioxy);
with the proviso that if Q¹ is Q² and X¹ is —O— then Q² must bear at least one substituent selected from C₂₋₅alkenyl, C₂₋₅alkynyl, C₁₋₄alkoxyC₁₋₄alkylaminoC₂₋₆alkanoyl, carbamoylC₁₋₆alkyl, C₁₋₄alkylcarbamoylC₁₋₆alkyl, and di(C₁₋₄alkyl)carbamoylC₁₋₆alkyl and optionally may bear a further 1 or 2 substituents as defined hereinbefore;
2) C₁₋₅alkylW¹Q² (wherein W¹ represents —O—, —S—, —SO—, —SO₂—, —C(O)—, —OC(O)—, —NQ³C(O)—, —C(O)NQ⁴-, —SO₂NQ⁵-, —NQ⁶S₂— or —NQ⁷- (wherein Q³, Q⁴, Q⁵, Q⁶ and Q⁷ each independently represents hydrogen, C₁₋₃alkyl, C₁₋₃alkoxyC₂₋₃alkyl, C₂-₅alkenyl, C₂₋₅alkyl or C₁₋₄haloalkyl) and Q² is as defined hereinbefore;
3) C₁₋₅alkylQ² (wherein Q² is as defined hereinbefore);
4) C₂₋₅alkenylQ² (wherein Q² is as defined hereinbefore);
5) C₂₋₅alkynylQ² (wherein Q² is as defined hereinbefore);
6) C₁₋₄alkylW²C₁₋₄alkylQ² (wherein W² represents —O—, —S—, —SO—, —SO₂—, —C(O)—, —OC(O)—NQ⁸C(O)—, —C(O)NQ⁹-, —SO₂NQ¹⁰-, —NQ¹¹SO₂— or —NQ¹²- (wherein Q⁸, Q⁹, Q¹⁰, Q¹¹ and Q¹² each independently represents hydrogen, C₁₋₃alkyl, C₁₋₃alkoxyC₂₋₃alkyl, C₂₋₅alkenyl, C₂₋₅alkynyl or C₁₋₄haloalkyl) and Q² is as defined hereinbefore);
7) C₂₋₅alkenylW²C₁₋₄alkylQ² (wherein W² and Q² are as defined hereinbefore);
8) C₂₋₅alkynylW²C₁₋₄alkylQ² (wherein W² and Q² are as defined hereinbefore);
9) C₁₋₄alkylQ¹³(C₁₋₄alkyl)ⱼ(W²)ₖQ¹⁴ (wherein W² is as defined hereinbefore, j is 0 or 1, k is 0 or 1, and Q¹³ and Q¹⁴ are each independently selected from hydrogen, C₁₋₃alkyl, cyclopentyl, cyclohexyl and a 5-6-membered saturated or partially unsaturated heterocyclic group with 1-2 heteroatoms, selected independently from O, S and N, which C₁₋₃alkyl group may bear 1 or 2 substituents selected from oxo, hydroxy, halogeno and C₁₋₄alkoxy and which cyclic group may bear 1, 2 or 3 substituents selected from C₂₋₅alkenyl, C₂₋₅alkynyl, C₁₋₆fluoroalkyl, C₁₋₆alkanoyl, aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, C₁₋₆fluoroalkanoyl, carbamoyl, C₁₋₄alkylcarbamoyl, di(C₁₋₄alkyl)carbamoyl, carbamoylC₁₋₆alkyl, C₁₋₄alkylcarbamoylC₁₋₆alkyl, di(C₁₋₄alkyl)carbamoylC₁₋₆alkyl, C₁₋₆alkylsulphonyl, C₁₋₆fluoroalkylsulphonyl, oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄alkyl, C₁₋₄alkylsulphonylC₁₋₄alkyl, C₁₋₄alkoxycarbonyl, C₁₋₄aminoalkyl, C₁₋₄alkylamino, di(C₁₋₄alkyl)amino, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g} ring D (wherein f is 0 or 1, g is 0 or 1 and ring D is a 5-6-membered saturated or partially unsaturated heterocyclic group with 1-2 heteroatoms, selected independently from O, S and N, which heterocyclic group may bear one or more substituents selected from C₁₋₄alkyl), with the provisos that Q¹³ cannot be hydrogen and one or both of Q¹³ and Q¹⁴ must be a 5-6-membered saturated or partially unsaturated heterocyclic group as defined hereinbefore which heterocyclic group bears at least one substituent selected from C₂₋₅alkenyl, C₂₋₅alkynyl, C₁₋₆alkanoyl, aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, C₁₋₆fluoroalkanoyl, carbamoyl, C₁₋₄alkylcarbamoyl, di(C₁₋₄alkyl)carbamoyl, carbamoylC₁₋₆alkyl, C₁₋₄alkylcarbamoylC₁₋₄alkyl, di(C₁₋₄alkyl)carbamoylC₁₋₆alkyl, C₁₋₆alkylsulphonyl and C₁₋₆fluoroalkylsulphonyl and which heterocyclic group optionally bears 1 or 2 further substituents selected from those defined hereinbefore); and
10) C₁₋₄alkylQ¹³C(O)-C₁₋₄alkylQ¹⁴ⁿ wherein Q¹³ is as defined hereinbefore and is not hydrogen and Q¹⁴ⁿ is a 5-6-membered saturated or partially unsaturated heterocyclic group containing at least one nitrogen atom and optionally containing a further heteroatom selected from N and O wherein Q¹⁴ⁿ is linked to C₁₋₆alkyl via a nitrogen atom and wherein Q¹⁴ⁿ optionally bears 1, 2 or 3 substituents selected from C₂₋₅alkenyl, C₂₋₅alkynyl, C₁₋₆fluoroalkyl, C₁₋₆alkanoyl, aminoC₂₋₆alkanoyl, C₁₋₄alkylaminoC₂₋₆alkanoyl, di(C-alkyl)aminoC₂₋₆alkanoyl, C₁₋₄alkoxyC₁₋₄alkylaminoC₂₋₆alkanoyl, C₁₋₆fluoroalkanoyl, carbamoyl, C₁₋₄alkylcarbamoyl, di(C₁₋₄alkyl)carbamoyl, carbamoylC₁₋₆alkyl, C₁₋₄alkylcarbamoylC₁₋₆alkyl, di(C₁₋₄alkyl)carbamoylC₁₋₆alkyl, C₁₋₆alkylsulphonyl, C₁₋₆fluoroalkylsulphonyl, oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄alkyl, C₁₋₄alkylsulphonylC₁₋₄alkyl, C₁₋₄alkoxycarbonyl, C₁₋₄aminoalkyl, C₁₋₄alkylamino, di(C₁₋₄alkyl)amino, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g} ring D (wherein f is 0 or 1, g is 0 or 1 and ring D is a 5-6-membered saturated or partially unsaturated heterocyclic group with 1-2 heteroatoms, selected independently from O, S and N, which heterocyclic group may bear one or more substituents selected from C₁₋₄alkyl)
or Q¹⁴ⁿ bears a single substituent selected from methylenedioxy and ethylenedioxy).
According to one aspect of the present invention R² is selected from:
Q¹X¹-
wherein X¹ is as defined hereinbefore and Q¹ is selected from one of the following nine groups:
1) Q² (wherein Q² is a 5-6-membered saturated or partially unsaturated heterocyclic group with 1-2 heteroatoms, selected independently from O, S and N, which heterocyclic group bears at least one substituent selected from C₂₋₅alkenyl, C₂₋₅alkynyl, aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, C₁₋₆fluoroalkanoyl, carbamoylC₁₋₆alkyl, C₁₋₄alkylcarbamoylC₁₋₆alkyl, di(C₁₋₄alkyl)carbamoylC₁₋₆alkyl, C₁₋₆alkylsulphonyl and C₁₋₆fluoroalkylsulphonyl and which heterocyclic group may optionally bear a further 1 or 2 substituents selected from C₂₋₅alkenyl, C₂₋₅alkynyl, C₁₋₆fluoroalkyl, C₁₋₆alkanoyl, aminoC₂₋₆alkanoyl C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, C₁₋₆fluoroalkanoyl, carbamoyl, C₁₋₄alkylcarbamoyl, di(C₁₋₄alkyl)carbamoyl, carbamoylC₁₋₆alkyl, C₁₋₄alkylcarbamoylC₁₋₆alkyl, di(C₁₋₄alkylcarbamoylC₁₋₆alkyl, C₁₋₆alkylsulphonyl, C₁₋₆fluoroalkylsulphonyl, oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄alkyl, C₁₋₄alkylsulphonylC₁₋₄alkyl, C₁₋₄alkoxycarbonyl, C₁₋₄aminoalkyl, C₁₋₄alkylamino, di(C₁₋₄alkyl)amino, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g} ring D (wherein f is 0 or 1, g is 0 or 1 and ring D is a 5-6-membered saturated or partially unsaturated heterocyclic group with 1-2 heteroatoms, selected independently from O, S and N, which cyclic group may bear one or more substituents selected from C₁₋₄alkyl),
or Q² bears a single substituent selected from methylenedioxy and ethylenedioxy);
with the proviso that if Q¹ is Q² and X¹ is —O— then Q² must bear at least one substituent selected from C₂₋₅alkenyl, C₂₋₅alkynyl, C₁₋₄alkoxyC₁₋₄alkylaminoC₂₋₆alkanoyl, carbamoylC₁₋₆alkyl, C₁₋₄alkylcarbamoylC₁₋₆alkyl, and di(C₁₋₄alkyl)carbamoylC₁₋₆alkyl and optionally may bear a further 1 or 2 substituents as defined hereinbefore;
2) C₁₋₅alkylW¹Q² (wherein W¹ represents —O—, —S—, —SO—, —SO₂—, —C(O)—, —OC(O)—, —NQ³C(O)—, —C(O)NQ⁴-, —SO₂NQ⁵-, —NQ⁶SO₂— or —NQ⁷- (wherein Q³, Q⁴, Q⁵, Q⁶ and Q⁷ each independently represents hydrogen, C₁₋₃alkyl, C₁₋₃alkoxyC₂₋₃alkyl, C₂₋₅alkenyl, C₂₋₅alkynyl or C₁₋₄haloalkyl) and Q² is as defined hereinbefore;
3) C₁₋₅alkylQ² (wherein Q² is as defined hereinbefore);
4) C₂₋₅alkenylQ² (wherein Q² is as defined hereinbefore),
5) C₂₋₅alkynylQ² (wherein Q² is as defined hereinbefore);
6) C₁₋₄alkylW²C₁₋₄alkylQ² (wherein W² represents —O—, —S—, —SO—, —SO₂—, —C(O)—, —OC(O)—NQ⁸C(O)—, —C(O)NQ⁹-, —SO₂NQ¹⁰, —NQ¹¹SO₂— or —NQ¹²- (wherein Q⁸, Q⁹, Q¹⁰, Q¹¹ and Q¹² each independently represents hydrogen, C₁₋₃alkyl C₁₋₃alkoxyC₂₋₃alkyl, C₂₋₅alkenyl, C₂₋₅alkynyl or C₁₋₄haloalkyl) and Q² is as defined hereinbefore);
7) C₂₋₅alkenylW²C₁₋₄alkylQ² (wherein W² and Q² are as defined hereinbefore);
8) C₂₋₅alkynylW²C₁₋₄alkylQ² (wherein W² and Q² are as defined hereinbefore); and
9) C₁₋₄alkylQ¹³(C₁₋₄alkyl)ⱼ(W²)ₖQ¹⁴ (wherein W² is as defined hereinbefore, j is 0 or 1, k is 0 or 1 and Q¹³ and Q¹⁴ are each independently selected from hydrogen, C₁₋₃alkyl, cyclopentyl, cyclohexyl and a 5-6-membered saturated or partially unsaturated heterocyclic group with 1-2 heteroatoms, selected independently from O, S and N, which C₁₋₃alkyl group may bear 1 or 2 substituents selected from oxo, hydroxy, halogeno and C₁₋₄alkoxy and which cyclic group may bear 1, 2 or 3 substituents selected from C₂₋₅alkenyl, C₂₋₅alkynyl, C₁₋₆fluoroalkyl, C₁₋₆alkanoyl , aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, C₁₋₆fluoroalkanoyl, carbamoyl, C₁₋₄alkylcarbamoyl, di(C₁₋₄alkyl)carbamoyl, carbamoylC₁₋₆alkyl, C₁₋₄alkylcarbamoylC₁₋₄alkyl, di(C₁₋₄alkyl)carbamoylC₁₋₆alkyl, C₁₋₆-alkylsulphonyl, C₁₋₆-fluoroalkylsulphonyl, oxo, hydroxy, halogeno, cyano, C₁₋₄cyanoalkyl, C₁₋₄alkyl, C₁₋₄-hydroxyalkyl, C₁₋₄alkoxy, C₁₋₄alkoxyC₁₋₄alkyl, C₁₋₄-alkylsulphonylC₁₋₄alkyl, C₁₋₄alkoxycarbonyl, C₁₋₄aminoalkyl, C₁₋₄alkylamino, di(C₁₋₄alkyl)amino, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g} ring D (wherein f is 0 or 1, g is 0 or 1 and ring D is a 5-6-membered saturated or partially unsaturated heterocyclic group with 1-2 heteroatoms, selected independently from O, S and N, which heterocyclic group may bear one or more substituents selected from C₁₋₄alkyl), with the provisos that Q¹³ cannot be hydrogen and one or both of Q¹³ and Q¹⁴ must be a 5-6-membered saturated or partially unsaturated heterocyclic group as defined hereinbefore which heterocyclic group bears at least one substituent selected from C₂₋₅alkenyl C₂₋₅alkyl, C₁₋₆alkanoyl aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, C₁₋₆fluoroalkanoyl, carbamoyl, C₁₋₄alkylcarbamoyl, di(C₁₋₄alkyl)carbamoyl, carbamoylC₁₋₄alkyl, C₁₋₄alkylcarbamoylC₁₋₆alkyl, di(C₁₋₄alkyl)carbamoylC₁₋₆alkyl, C₁₋₆alkylsulphonyl and C₁₋₄fluoroalkylsulphonyl and which heterocyclic group optionally bears 1 or 2 further substituents selected from those defined hereinbefore).

According to one aspect of the present invention R² is selected from:
Q¹X¹-
wherein X¹ is as defined hereinbefore and Q¹ is selected from one of the following eight groups:
1) Q² (wherein Q² is a 5-6-membered saturated or partially unsaturated heterocyclic group with 1-2 heteroatoms, selected independently from O, S and N, which heterocyclic group bears at least one substituent selected from C₂₋₅alkenyl, C₂₋₅alkynyl, aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, C₁₋₄fluoroalkanoyl, carbamoylC₁₋₆alkyl, C₁₋₄alkylcarbamoylC₁₋₆alkyl, di(C₁₋₄alkyl)carbamoylC₁₋₆alkyl, C₁₋₆alkylsulphonyl and C₁₋₆fluoroalkylsulphonyl and which heterocyclic group may optionally bear a further 1 or 2 substituents selected from C₂₋₅alkenyl, C₂₋₅alkynyl, C₁₋₆fluoroalkyl, C₁₋₆alkanoyl, aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, C₁₋₆fluoroalkanoyl, carbamoyl, C₁₋₄alkylcarbamoyl, di(C₁₋₄alkyl)carbamoyl, carbamoylC₁₋₆alkyl, C₁₋₄alkylcarbamoylC₁₋₆alkyl, di(C₁₋₄alkyl)carbamoylC₁₋₆alkyl, C₁₋₆alkylsulphonyl, C₁₋₆fluoroalkylsulphonyl, oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄alkyl, C₁₋₄alkylsulphonylC₁₋₄alkyl, C₁₋₄alkoxycarbonyl, C₁₋₄aminoalkyl, C₁₋₄alkylamino, di(C₁₋₄alkyl)amino, C₁₋₄alkylaminoC₁₋₄alkyl, di(C₁₋₄alkyl)aminoC₁₋₄alkyl, C₁₋₄alkyl, C₁₋₄alkoxy, di(C₁₋₄alkyl)aminoC₁₋₄alkoxy and a group —(—O—)_{f}(C₁₋₄alkyl)_{g} ring D (wherein f is 0 or 1, g is 0 or 1 and ring D is a 5-6-membered saturated or partially unsaturated heterocyclic group with 1-2 heteroatoms, selected independently from O, S and N, which cyclic group may bear one or more substituents selected from C₁₋₄alkyl),
or Q² bears a single substituent selected from methylenedioxy and ethylenedioxy); with the proviso that if Q¹ is Q² and X¹ is —O— then Q² must bear at least one substituent selected from C₂₋₅alkenyl, C₂₋₅alkyl, C₁₋₄alkoxyC₁₋₄alkylaminoC26alkanoyl, carbamoylC₁₋₆alkyl, C₁₋₄alkylcarbamoylC₁₋₆alkyl, and di(C₁₋₄alkyl)carbamoylC₁₋₆alkyl and optionally may bear a further 1 or 2 substituents as defined hereinbefore;
2) C₁₋₅alkylW¹Q² (wherein W¹ represents —O—, —S—, —SO—, —SO₂—, —C(O)—, —OC(O)—, —NQ³C(O)—, —C(O)NQ⁴-, —SO₂NQ⁵-, —NQ⁶SO₂— or —NQ⁷- (wherein Q³, Q⁴, Q⁵, Q⁶ and Q⁷ each independently represents hydrogen, C₁₋₃alkyl, C₁₋₃alkoxyC₂₋₃alkyl, C₂₋₅alkenyl, C₂₋₅alkynyl or C₁₋₄alkenyl) and Q² is as defined hereinbefore;
3) C₁₋₅alkylQ² (wherein Q² is as defined hereinbefore);
4) C₂₋₅alkenylQ² (wherein Q² is as defined hereinbefore);
5) C₂₋₅alkynylQ² (wherein Q² is as defined hereinbefore);
6) C₁₋₄alkylW²C₁₋₄alkylQ² (wherein W² represents —O—, —S—, —SO—, —SO₂—, —C(O)—, —OC(O)—NQ⁸C(O)—, —C(O)NQ⁹-, —SO₂NQ¹⁰-, —NQ¹¹SO₂— or —NQ¹²- (wherein Q⁸, Q⁹, Q¹⁰, Q¹¹ and Q¹² each independently represents hydrogen, C₁₋₃alkyl, C₁₋₃alkoxyC₂₋₃alkyl, C₂₋₅alkenyl, C₂₋₅alkynyl or C₁₋₄haloalkyl) and Q² is as defined hereinbefore);
7) C₂₋₅alkenylW²C₁₋₄alkylQ² (wherein W² and Q² are as defined hereinbefore); and
8) C₂₋₅alkynylW²C₁₋₄alkylQ² (wherein W² and Q² are as defined hereinbefore).

According to one aspect of the present invention there is provided a compound of the formula I as defined hereinbefore
wherein Z, R¹ and R³ are as defined hereinbefore and
R² is Q¹X¹-
wherein X¹ represents —O—, —S— or —NR⁴— wherein R⁴ is hydrogen, C₁₋₃alkyl or C₁₋₃alkoxyC₂₋₃alkyl and Q¹ is selected from one of the following ten groups:
1) Q² (wherein Q² is a 5-6-membered saturated or partially unsaturated heterocyclic group with 1-2 heteroatoms, selected independently from O, S and N, which heterocyclic group bears at least one substituent selected from aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, carbamoylC₁₋₆alkyl, C₁₋₄alkylcarbamoylC₁₋₆alkyl and di(C₁₋₄alkyl)carbamoylC₁₋₆alkyl and which heterocyclic group may optionally bear a further 1 or 2 substituents selected, from C₂₋₅alkenyl, C₂₋₅alkynyl, C₁₋₆fluoroalkyl, C₁₋₆alkanoyl, aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, C₁₋₆fluoroalkanoyl, carbamoyl, C₁₋₄alkylcarbamoyl, di(C₁₋₄alkyl)carbamoyl, carbamoylC₁₋₄-alkyl, C₁₋₄alkylcarbamoylC₁₋₆alkyl, di(C₁₋₄alkylcarbamoylC₁₋₆alkyl, C₁₋₆alkylsulphonyl, C₁₋₆fluoroalkylsulphonyl, oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄alkyl, C₁₋₄alkylsulphonylC₁₋₄alkyl, C₁₋₄alkoxycarbonyl, C₁₋₄aminoalkyl, C₁₋₄alkylamino, di(C₁₋₄alkyl)amino, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g} ring D (wherein f is 0 or 1, g is 0 or 1 and ring D is a 5-6-membered saturated or partially unsaturated heterocyclic group with 1-2 heteroatoms, selected independently from O, S and N, which cyclic group may bear one or more substituents selected from C₁₋₄alkyl),
or Q² bears a single substituent selected from methylenedioxy and ethylenedioxy);
with the proviso that if Q¹ is Q² and X¹ is —O— then Q² must bear at least one substituent selected from C₁₋₄alkoxyC₁₋₄alkylaminoC₂₋₆alkanoyl, carbamoylC₁₋₆alkyl, C₁₋₄alkylcarbamoylC₁₋₆alkyl, and di(C₁₋₄alkyl)carbamoylC₁₋₆alkyl and optionally may bear a further 1 or 2 substituents as defined hereinbefore;
2) C₁₋₅alkylW¹Q² (wherein W¹ represents —O—, —S—, —SO—, —SO₂—, —C(O)—, —OC(O)—, —NQ³C(O)—, —C(O)NQ⁴-, —SO₂NQ⁵-, —NQ⁶SO₂— or —NQ⁷- (wherein Q³, Q⁴, Q⁵, Q⁶ and Q⁷ each independently represents hydrogen, C₁₋₃alkyl, C₁₋₃alkoxyC₂₋₃alkyl, C₂₋₅alkenyl, C₂₋₅alkynyl or C₁₋₄haloalkyl) and Q² is as defined hereinbefore;
3) C₁₋₅alkylQ² (wherein Q² is as defined hereinbefore);
4) C₂₋₅alkenylQ² (wherein Q² is as defined hereinbefore);
5) C₂₋₅alkynylQ² (wherein Q² is as defined hereinbefore);
6) C₁₋₄alkylW²C₁₋₄alkylQ² (wherein W² represents —O—, —S—, —SO—, —SO₂—, —C(O)—, —OC(O)—NQ⁸C(O)—, —C(O)NQ⁹-, —SO₂NQ¹⁰, —NQ¹¹SO₂— or —NQ¹²- (wherein Q⁸, Q⁹, Q¹⁰, Q¹¹ and Q¹² each independently represents hydrogen, C₁₋₃alkyl, C₁₋₃alkoxyC₂₋₃alkyl, C₂₋₅alkenyl, C₂₋₅alkynyl or C₁₋₄haloalkyl) and Q² is as defined hereinbefore);
7) C₂₋₅alkenylW²C₁₋₄alkylQ² (wherein W² and Q² are as defined hereinbefore);
8) C₂₋₅alkynylW²C₁₋₄alkylQ² (wherein W² and Q² are as defined hereinbefore);
9) C₁₋₄alkylQ¹³(C₁₋₄alkyl)ⱼ(W²)ₖQ¹⁴ (wherein W² is as defined hereinbefore, j is 0 or 1, k is 0 or 1, and Q¹³ and Q¹⁴ are each independently a 5-6-membered saturated or partially unsaturated heterocyclic group with 1-2 heteroatoms, selected independently from O, S and N, which heterocyclic group may bear 1, 2 or 3 substituents selected from C₂₋₅alkenyl, C₂₋₅alkynyl, C₁₋₆fluoroalkyl, C₁₋₆alkanoyl, aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, C₁₋₆fluoroalkanoyl, carbamoyl, C₁₋₄alkylcarbamoyl, di(C₁₋₄alkyl)carbamoyl, carbamoylC₁₋₆alkyl, C₁₋₄alkylcarbamoylC₁₋₆alkyl, di(C₁₋₄alkyl)carbamoylC₁₋₆alkyl, C₁₋₆alkylsulphonyl, C₁₋₆fluoroalkylsulphonyl, oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄alkyl, C₁₋₄alkylsulphonylC₁₋₄alkyl, C₁₋₄alkoxycarbonyl, C₁₋₄aminoalkyl, C₁₋₄alkylamino, di(C₁₋₄alkyl)amino, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g} ring D (wherein f is 0 or 1, g is 0 or 1 and ring D is a 5-6-membered saturated or partially unsaturated heterocyclic group with 1-2 heteroatoms, selected independently from O, S and N, which heterocyclic group may bear one or more substituents selected from C₁₋₄alkyl), with the proviso that one or both of Q¹³ and Q¹⁴ bears at least one substituent selected from aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, carbamoylC₁₋₆alkyl, C₁₋₄alkylcarbamoylC₁₋₆alkyl and di(C₁₋₄alkyl)carbamoylC₁₋₆alkyl, and which heterocyclic group optionally bears 1 or 2 further substituents selected from those defined hereinbefore); and
10) C₁₋₄alkylQ¹³-C(O)-C₁₋₄alkylQ¹⁴ⁿ wherein Q¹³ is as defined hereinbefore and Q¹⁴ⁿ is a 5-6-membered saturated or partially unsaturated heterocyclic group containing at least one nitrogen atom and optionally containing a further heteroatom selected from N and O wherein Q¹⁴ⁿ is linked to C₁₋₆alkyl via a nitrogen atom or a carbon atom and wherein Q¹⁴ⁿ optionally bears 1, 2 or 3 substituents selected from C₂₋₅alkenyl, C₂₋₅alkynyl, C₁₋₆fluoroalkyl, C₁₋₆alkanoyl, aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, C₁₋₄fluoroalkanoyl, carbamoyl, C₁₋₄alkylcarbamoyl, di(C₁₋₄alkyl)carbamoyl, carbamoylC₁₋₆alkyl, C₁₋₄alkylcarbamoylC₁₋₆alkyl, di(C₁₋₄alkyl)carbamoylC₁₋₆alkyl, C₁₋₆alkylsulphonyl, C₁₋₆fluoroalkylsulphonyl, oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄alkyl, C₁₋₄alkylsulphonylC₁₋₄alkyl, C₁₋₄alkoxycarbonyl, C₁₋₄aminoalkyl, C₁₋₄alkylamino; di(C₁₋₄alkyl)amino, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g} ring D (wherein f is 0 or 1, g is 0 or 1 and ring D is a 5-6-membered saturated or partially unsaturated heterocyclic group with 1-2 heteroatoms, selected independently from O, S and N, which heterocyclic group may bear one or more substituents selected from C₁₋₄alkyl)
or Q¹⁴ⁿ bears a single substituent selected from methylenedioxy and ethylenedioxy);
   or a salt thereof or a prodrug thereof.

According to another aspect of the present invention there is provided a compound according to formula I of the formula Ia: 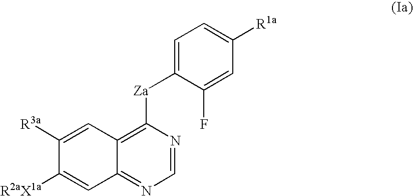
wherein:
Za is —NH—, —O— or —S—;
R^{1a} represents bromo or chloro;
R^{3a} represents C₁₋₃alkoxy or hydrogen;
X^{1a} represents —O—, —S— or —NR^{4a}—wherein R^{4a} is hydrogen, C₁₋₃alkyl or C₁₋₃alkoxyC₂₋₃alkyl;
R^{2a} is selected from one of the following groups:
1) C₁₋₅alkylR^{5a} (wherein R^{5a} is a 5- or 6-membered heterocyclic ring selected from morpholine, pyrrolidine, piperidine and piperazine which heterocyclic zing bears at least one substituent selected from aminoC₂₋₄alkanoyl, C₁₋₄alkylaminoC₂₋₄alkanoyl, di(C₁₋₄alkyl)aminoC₂₋₄alkanoyl, C₁₋₄alkoxylC₁₋₄alkylaminoC₂₋₄alkanoyl, methylenedioxy and ethylenedioxy);
2) C₂₋₅alkenylR^{5a} (wherein R^{5a} is as defined hereinbefore);
3) C₂₋₅alkynylR^{5a} (wherein R^{5a} is as defined hereinbefore);
4) C₁₋₅alkylR^{6a}C(O)(CH₂)ₘₐR^{7a} (wherein ma is 1 or 2, R^{6a} is a 5- or 6-membered heterocyclic ring selected from morpholine, pyrrolidine, piperidine and piperazine which heterocyclic ring may bear one or two substituents selected from fluoro, hydroxy and methyl and R^{7a} is a 5- or 6-membered heterocyclic ring selected from pyrrolidine, piperidine, piperazine and morpholine which heterocyclic ring is linked to (CH₂)ₘₐ via a nitrogen atom or a carbon atom and which heterocyclic ring may bear one or more substituents selected from hydroxy, halogeno, C₁₋₄alkanoyl, methylenedioxy and ethylenedioxy); and
5) C₁₋₅alkylR^{6a} (CH₂)ₘₐC(O)R^{8a} (wherein ma and R^{6a} are as defined hereinbefore and R^{8a} is a 5- or 6-membered heterocyclic ring selected from pyrrolidine, piperidine, piperazine and morpholine which heterocyclic ring is linked to C(O) via a nitrogen atom or a carbon atom and which heterocyclic ring may bear one or more substituents selected from hydroxy, halogeno, C₁₋₄alkanoyl, methylenedioxy and ethylenedioxy)
   or a salt thereof.

According to another aspect of the present invention there is provided a compound according to formula I of the formula Ia: 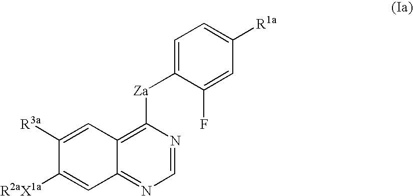
wherein:
Za, R^{1a}, R^{3a} and X^{1a} are as described hereinbefore and
R^{2a} is selected from one of the following groups:
1) C₁₋₅alkylR^{5a} (wherein R^{5a} is a 5- or 6-membered heterocyclic ring selected from morpholine, pyrrolidine, piperidine and piperazine which heterocyclic ring bears at least one substituent selected from aminoC₂₋₄alkanoyl, C₁₋₄alkylaminoC₂₋₄alkanoyl, di(C₁₋₄alkyl)aminoC₂₋₄alkanoyl, C₁₋₄alkoxyC₁₋₄alkylaminoC₂₋₄alkanoyl methylenedioxy and ethylenedioxy);
2) C₂₋₅alkenylR^{5a} (wherein R^{5a} is as defined hereinbefore);
3) C₂₋₅alkynylR^{5a} (wherein R^{5a} is as defined hereinbefore); and
4) C₁₋₅alkylR^{6a}C(O)(CH₂)ₘₐR^{7a} (wherein ma is 1 or 2, R^{6a} is a 5- or 6-membered heterocyclic ring selected from morpholine, pyrrolidine, piperidine and piperazine which heterocyclic ring may bear one or two substituents selected from fluoro,.hydroxy and methyl, and R^{7a} is a 5- or 6-membered heterocyclic ring selected from pyrrolidine, piperidine, piperazine and morpholine which heterocyclic ring is linked to (CH₂)ₘₐ via a nitrogen atom or a carbon atom and which heterocyclic ring may bear one or more substituents selected from hydroxy, halogeno, C₁₋₄alkanoyl, methylenedioxy and ethylenedioxy);
   or a salt thereof.

According to another aspect of the present invention there is provided a compound according to formula I of the formula Ia: 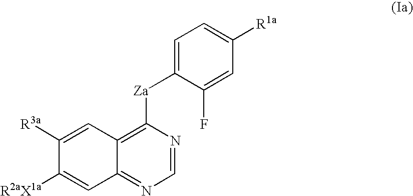
wherein:
Za, R^{1a}, R^{3a} and X^{1a} are as described hereinbefore and
R^{2a} is selected from one of the following groups:
1) C₁₋₅alkylR^{5a} (wherein R^{5a} is a 5- or 6-membered heterocyclic ring selected from morpholine, pyrrolidine, piperidine and piperazine which heterocyclic ring bears at least one substituent selected from aminoC₂₋₄alkanoyl, C₁₋₄alkylaminoC₂₋₄alkanoyl, di(C₁₋₄alkyl)aminoC₂₋₄alkanoyl, C₁₋₄alkoxyC₁₋₄alkylaminoC₂₋₄alkanoyl, methylenedioxy and ethylenedioxy);
2) C₂₋₅-alkenylR^{5a} (wherein R^{5a} is as defined hereinbefore);
3) C₂₋₅alkynylR^{5a} (wherein R^{5a} is as defined hereinbefore); and
4) C₁₋₅alkylR^{6a}C(O)(CH₂)ₘₐR^{7a} (wherein ma is 1 or 2, R^{6a} is a 5- or 6-membered heterocyclic ring selected from morpholine, pyrrolidine, piperidine and piperazine which heterocyclic ring may bear one or two substituents selected from fluoro, hydroxy and methyl, and R^{7a} is a 5- or 6-membered heterocyclic ring selected from pyrrolidine, piperidine, piperazine and morpholine which heterocyclic ring is linked to (CH₂)ₘₐ via a nitrogen atom and which heterocyclic ring may bear one or more substituents selected from hydroxy, halogeno, C₁₋₄alkanoyl, methylenedioxy and ethylenedioxy);
   or a salt thereof.

According to another aspect of the present invention there is provided a compound according to formula I of the formula Ia: 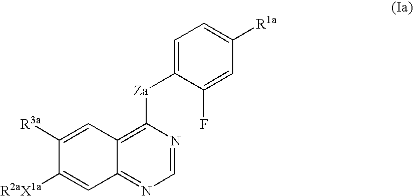
wherein:
Za, R^{1a}, R^{3a} and X^{1a} are as described hereinbefore and
R^{2a} is selected from one of the following groups:
1) C₁₋₅alkylR^{5a} (wherein R^{5a} is a 5- or 6-membered heterocyclic ring selected from morpholine, pyrrolidine, piperidine and piperazine which heterocyclic ring bears at least one substituent selected from aminoC₂₋₄alkanoyl, C₁₋₄alkylaminoC₇₋₄alkanoyl, di(C₁₋₄alkyl)aminoC₂₋₄alkanoyl, C₁₋₄alkoxyC₁₋₄alkylaminoC₂₋₄alkanoyl, methylenedioxy and ethylenedioxy);
2) C₂₋₅alkenylR^{5a} (wherein R^{5a} is as defined hereinbefore); and
3) C₂₋₅alkylR^{5a} (wherein R^{5a} is as defined hereinbefore);
   or a salt thereof.

According to one aspect of the present invention R^{2a} is C₁₋₅alkylR^{5a} (wherein R^{5a} is a 5- or 6membered heterocyclic ring selected from morpholine, pyrrolidine, piperidine and piperazine which heterocyclic ring bears at least one substituent selected from aminoC₂₋₄alkanoyl, C₁₋₄alkylaminoC₂₋₄alkanoyl, di(C₁₋₄alkyl)aminoC₂₋₄alkanoyl, C₁₋₄alkoxyC₁₋₄alkylaminoC₂₋₄alkanoyl, methylenedioxy and ethylenedioxy).

According to one aspect of the present invention R^{2a} is C₁₋₅alkylR^{6a}C(O)(CH₂)ₘₐR^{7a} (wherein ma is 1 or 2, R^{6a} is a 5- or 6-membered heterocyclic ring selected from morpholine, pyrrolidine, piperidine and piperazine which heterocyclic ring may bear one or two substituents selected from fluoro, hydroxy and methyl, and R^{7a} is a 5- or 6-membered heterocyclic ring selected from pyrrolidine, piperidine, piperazine and morpholine which heterocyclic ring is linked to (CH₂)ₘₐ via a nitrogen atom or a carbon atom and which heterocyclic ring may bear one or more substituents selected from hydroxy, halogeno, C₁₋₄alkanoyl, methylenedioxy and ethylenedioxy).

According to one aspect of the present invention Za is —NH—.

According to one aspect of the present invention R^{3a} is methoxy.

According to one aspect of the present invention X^{1a} is —O—;

According to another aspect of the present invention there is provided a compound of the formula Ib: 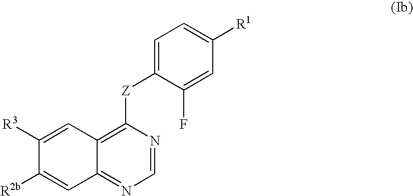
wherein:
Z, R¹ and R³ are as defined hereinbefore and
R^{2b} is selected from one of the following three groups:
(i) Q^{1b}X¹-
wherein X¹ is as defined hereinbefore and Q^{1b} is selected from one of the following ten groups:
1) Q^{2b} (wherein Q^{2b} is a 5-6-membered saturated or partially unsaturated heterocyclic group with 1-2 heteroatoms, selected independently from O, S and N, which heterocyclic group bears at least one substituent selected from C₂₋₅alkenyl, C₂₋₅alkynyl, C₁₋₆fluoroalkyl, aminoC₂₋₆alkanoyl, C₁₋₄alkylaminoC₂₋₆alkanoyl di(C₁₋₄alkyl)aminoC₂₋₆alkanoyl, C₁₋₄alkoxyC₁₋₄alkylaminoC₂₋₆alkanoyl, C₂₋₆fluoroalkanoyl, carbamoylC₁₋₆alkyl, C₁₋₆alkylcarbamoylC₁₋₆alkyl, di(C₁₋₄alkyl)carbamoylC₁₋₆alkyl and C₁₋₆fluoroalkylsulphonyl and which heterocyclic group may optionally bear a further 1 or 2 substituents selected from C₂₋₅alkenyl, C₂₋₅alkynyl, C₁₋₆fluoroalkyl, C₁₋₆alkanoyl, aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, C₁₋₆fluoroalkanoyl, carbamoyl, C₁₋₄alkylcarbamoyl, di(C₁₋₄alkyl)carbamoyl, carbamoylC₁₋₆alkyl, C₁₋₄alkylcarbamoylC₁₋₆alkyl, di(C₁₋₄alkyl)carbamoylC₁₋₆alkyl, C₁₋₆alkylsulphonyl, C₁₋₆fluoroalkylsulphonyl, oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄alkyl, C₁₋₄alkylsulphonylC₁₋₄alkyl, C₁₋₄alkoxycarbonyl, C₁₋₄aminoalkyl, C₁₋₄alkylamino, di(C₁₋₄alkyl)amino, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g} ring D (wherein fis 0 or 1, g is 0 or 1 and ring D is a 5-6-membered saturated or partially unsaturated heterocyclic group with 1-2 heteroatoms, selected independently from O, S and N, which cyclic group may bear one or more substituents selected from C₁₋₄alkyl),
or Q^{2b} bears a single substituent selected from methylenedioxy and ethylenedioxy);
with the proviso that if Q^{1b} is Q^{2b} and X¹ is —O— then Q^{2b} must bear at least one substituent selected from C₂₋₅alkenyl, C₂₋₅alkynyl, C₁₋₄alkoxyC₁₋₄alkylaminoC₂₋₆alkanoyl, carbamoylC₁₋₆alkyl, C₁₋₄alkylcarbamoylC₁₋₆alkyl, and di(C₁₋₄alkyl)carbamoylC₁₋₆alkyl and optionally may bear a further 1 or 2 substituents as defined hereinbefore;
2) C₁₋₅alkylW¹Q² (wherein W¹ and Q² are as defined hereinbefore);
3) C₁₋₅alkylQ^{2b} (wherein Q^{2b} is as defined hereinbefore);
4) C₂₋₅alkenylQ² (wherein Q² is as defined hereinbefore);
5) C₂₋₅alkynylQ² (wherein Q² is as defined hereinbefore);
6) C₁₋₄alkylW²C₁₋₄alkylQ² (wherein W² and Q² are as defined hereinbefore);
7) C₂₋₅alkenylW²C₁₋₄alkylQ² (wherein W² and Q² are as defined hereinbefore);
8) C₂₋₅alkynylW²C₁₋₄alkylQ² (wherein W² and Q² are as defined hereinbefore);
9) C₁₋₄alkylQ^{13b}(C₁₋₄alkyl)ⱼ(W²)ₖQ^{14b} (wherein W² is as defined hereinbefore, j is 0 or 1, k is 0 or 1, and Q^{13b} and Q^{14b} are each independently selected from hydrogen, C₁₋₃alkyl, cyclopentyl, cyclohexyl and a 5-6-membered saturated or partially unsaturated heterocyclic group with 1-2 heteroatoms, selected independently from O, S and N, which C₁₋₃alkyl group may bear 1 or 2 substituents selected from oxo, hydroxy, halogeno and C₁₋₄alkoxy and which cyclic group may bear 1, 2 or 3 substituents selected from C₂₋₅alkenyl, C₂₋₅alkynyl, C₁₋₆fluoroalkyl, C₁₋₆alkanoyl, aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, C₁₋₆fluoroalkanoyl, carbamoyl, C₁₋₄alkylcarbamoyl, di(C₁₋₄alkyl)carbamoyl, carbamoylC₁₋₆alkyl, C₁₋₄alkylcarbamoylC₁₋₆alkyl, di(C₁₋₄alkyl)carbamoylC₁₋₆alkyl, C₁₋₆alkylsulphonyl, C₁₋₆fluoroalkylsulphonyl, oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄alkyl, C₁₋₄alkylsulphonylC₁₋₄alkyl, C₁₋₄alkoxycarbonyl, C₁₋₄aminoalkyl, C₁₋₄alkylamino, di(C₁₋₄alkyl)amino, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g} ring D (wherein f is 0 or 1, g is 0 or 1 and ring D is a 5-6-membered saturated or partially unsaturated heterocyclic group with 1-2 heteroatoms, selected independently from O, S and N, which heterocyclic group may bear one or more substituents selected from C₁₋₄alkyl), with the provisos that Q^{13b} cannot be hydrogen and one or both of Q^{13b} and Q^{14b} must be a 5-6-membered saturated or partially unsaturated heterocyclic group as defined hereinbefore which heterocyclic group bears at least one substituent selected from C₂₋₅alkenyl, C₂₋₅alkynyl, C₁₋₆fluoroalkyl, aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, C₁₋₆fluoroalkanoyl, carbamoyl, C₁₋₄alkylcarbamoyl, di(C₁₋₄alkyl)carbamoyl, carbamoylC₁₋₆alkyl, C₁₋₄alkylcarbamoylC₁₋₆alkyl, di(C₁₋₄alkylcarbamoylC₁₋₆alkyl and C₁₋₆fluoroalkylsulphonyl and which heterocyclic group optionally bears 1 or 2 further substituents selected from those defined hereinbefore); and
10) C₁₋₄alkylQ¹³-C(O)-C₁₋₄alkylQ¹⁴ⁿ (wherein Q¹³ and Q¹⁴ⁿ are as defined hereinbefore);
(ii) Q¹⁵W³— (wherein W³ and Q¹⁵ are defined hereinbefore); and
(iii) Q²¹W⁴C₁₋₅alkylX¹ (wherein X¹, W⁴ and Q²¹ are as defined hereinbefore);
   or a salt thereof or a prodrug thereof.
According to another aspect of the present invention R^{2b} is selected from:
Q^{1b}X¹-
   wherein X¹ is as defined hereinbefore and Q^{1b} is selected from one of the following ten groups:
1) Q^{2b} (wherein Q^{2b} is a 5-6membered saturated or partially unsaturated heterocyclic group with 1-2 heteroatoms, selected independently from O, S and N, which heterocyclic group bears at least one substituent selected from C₂₋₅alkenyl, C₂₋₅alkyl, aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, C₁₋₆fluoroalkanoyl, carbamoylC₁₋₆alkyl, C₁₋₄alkylcarbamoylC₁₋₆alkyl and di(C₁₋₄alkyl)carbamoylC₁₋₆alkyl and which heterocyclic group may optionally bear a further 1 or 2 substituents selected from C₂₋₅alkenyl, C₂₋₅alkynyl, C₁₋₆fluoroalkyl C₁₋₆-alkanoyl, aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, C₁₋₆fluoroalkanoyl, carbamoyl, C₁₋₄alkylcarbamoyl, di(C₁₋₄alkyl)carbamoyl, carbamoylC₁₋₆alkyl, C₁₋₄alkylcarbamoylC₁₋₆alkyl, di(C₁₋₄alkyl)carbamoylC₁₋₆alkyl, C₁₋₆alkylsulphonyl, C₁₋₆fluoroalkylsilphonyl, oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄alkyl, C₁₋₄alkylsulphonylC₁₋₄alkyl, C₁₋₄alkoxycarbonyl, C₁₋₄aminoalkyl, C₁₋₄alkylamino, di(C₁₋₄alkyl)amino, C₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g} ring D (wherein f is 0 or 1, g is 0 or 1 and ring D is a 5-6-membered saturated or partially unsaturated heterocyclic group with 1-2 heteroatoms, selected independently from O, S and N, which cyclic group may bear one or more substituents selected from C₁₋₄alkyl),
or Q^{2b} bears a single substituent selected from methylenedioxy and ethylenedioxy);
with the proviso that if Q^{1b} is Q^{2b} and X¹ is —O— then Q^{2b} must bear at least one substituent selected from C₂₋₅alkenyl, C₂₋₅alkynyl C₁₋₄alkoxyC₁₋₄alkylaminoC₂₋₆alkanoyl, carbamoylC₁₋₆alkyl, C₁₋₄alkylcarbamoylC₁₋₆alkyl, and di(C₁₋₄alkyl)carbamoylC₁₋₆alkyl and optionally may bear a further 1 or 2 substituents as defined hereinbefore;
2) C₁₋₅alkylW¹Q^{2b} (wherein W¹ and Q^{2b} are as defined hereinbefore);
3) C₁₋₅alkylQ^{2b} (herein Q^{2b} is as defined hereinbefore);
4) C₂₋₅alkenylQ^{2b} (wherein Q^{2b} is as defined hereinbefore);
5) C₂₋₅alkylQ^{2b} (wherein Q^{2b} is as defined hereinbefore);
6) C₁₋₄alkylW²C₁₋₄alkylQ^{2b} (wherein W² and Q^{2b} are as defined hereinbefore);
7) C₂₋₅alkenylW²C₁₋₄alkylQ^{2b} (wherein W² and Q^{2b} are as defined hereinbefore);
8) C₂₋₅alkynylW²C₁₋₄alkylQ^{2b} (wherein W² and Q^{2b} are as defined hereinbefore);
9) C₁₋₄alkylQ^{13b}(C₁₋₄alkyl)ⱼ(W²)ₖQ^{14b} (wherein W² is as defined hereinbefore, j is 0 or 1, k is 0 or 1, and Q^{13b} and Q^{14b} are each independently selected from hydrogen, C₁₋₃alkyl, cyclopentyl, cyclohexyl and a 5-6-membered saturated or partially unsaturated heterocyclic group with 1-2 heteroatoms, selected independently from O, S and N, which C₁₋₃alkyl group may bear 1 or 2 substituents selected from oxo, hydroxy, halogeno and C₁₋₄alkoxy and which cyclic group may bear 1, 2 or 3 substituents selected from C₂₋₅alkenyl, C₂₋₅alkynyl, C₁₋₆fluoroalkyl, C₁₋₆alkanoyl, aminoC₂₋₆alkanoyl, C₁₋₄alkylaminoC₂₋₆alkanoyl, di(C₁₋₄alkyl)aminoC₂₋₆alkanoyl, C₁4alkoxyC₁₋₄alkylaminoC2-alkanoyl, C₁-fluoroalkanoyl, carbamoyl, C₁₋₄alkylcarbamoyl, di(C₁₋₄alkyl)carbamoyl, carbamoylC₂₋₆alkyl, C₁₋₄alkylcarbamoylC₁₋₄alkyl, di(C₁₋₄alkyl)carbamoylC₁₋₆alkyl, C₁₋₆alkylsulphonyl, C₁₋₆fluoroalkylsulphouyl, oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄alkyl, C₁₋₄alkylsulphonylC₁₋₄alkyl, C₁₋₄alkoxycarbonyl, C₁₋₄aminoalkyl, C₁₋₄alkylamino, di(C₁₋₄alkyl)amino, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g} ring D (wherein f is 0 or 1, g is 0 or 1 and ring D is a 5-6-membered saturated or partially unsaturated heterocyclic group with 1-2 heteroatoms, selected independently from O, S and N, which heterocyclic group may bear one or more substituents selected from C₁₋₄alkyl), with the provisos that Q^{13b} cannot be hydrogen and one or both of Q^{13b} and Q^{14b} must be a 5-6-membered saturated or partially unsaturated heterocyclic group as defined hereinbefore which heterocyclic group bears at least one substituent selected from C₂₋₅alkenyl, C₂₋₅alkynyl, aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, carbamoyl, C₁₋₄alkylcarbamoyl, di(C₁₋₄alkyl)carbamoyl, carbamoylC₁₋₆alkyl, C₁₋₄alkylcarbamoylC₁₋₆alkyl and di(C₁₋₄alkyl)carbamoylC₁₋₆alkyl and which heterocyclic group optionally bears 1 or 2 further substituents selected from those defined hereinbefore); and
10) C₁₋₄alkylQ¹³-C(O)-C₁₋₄alkylQ¹⁴ⁿ wherein Q¹³ is as defied hereinbefore and is not hydrogen and Q¹⁴ⁿ is a 5-6-membered saturated or partially unsaturated heterocyclic group containing at least one nitrogen atom and optionally containing a further heteroatom selected from N and O wherein Q¹⁴ⁿ is linked to C₁₋₄alkyl via a nitrogen atom and wherein Q¹⁴ⁿ optionally bears 1, 2 or 3 substituents selected from C₂₋₅alkenyl, C₂₋₅alkynyl, C₁₋₆fluoroalkyl C₁₋₆alkanoyl, aminoC₂₋₆alkanoyl, C₁₋₄alkylaminoC₂₋₆alkanoyl, di(C₁₋₄alkyl)aminoC₂₋₆alkanoyl C₁₋₄alkoxyC₁₋₄alkylaminoC₂₋₆alkoxy, C₁₋₆fluoroalkanoyl, carbamoyl, C₁₋₄alkylcarbamoyl, di(C₁₋₄alkyl)carbamoyl, carbamoylC₁₋₆alkyl, C₁₋₄alkylcarbamoylC₁₋₆alkyl, di(C₁₋₄alkyl)carbamoylC₁₋₆alkyl, C₁₋₆alkylsulphonyl, C₁₋₆-fluoroalkylsulphonyl, oxo, hydroxy, halogeno, cyano, C₁₋₄cyanoalkyl, C₁₋₄alkyl, C₁₋₄hydroxyalkyl, C₁₄-alkoxy, C₁₋₄alkoxyC₁₋₄alkyl, C₁₋₄alkylsulphonylC₁₋₄alkyl, C₁₋₄alkoxycarbonyl, C₁₋₄aminoalkyl, C₁₋₄alkylamino, di(C₁₋₄alkyl)amino, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄l)_{g} ring D (wherein f is 0 or 1, g is 0 or 1 and ring D is a 5-6-membered saturated or partially unsaturated heterocyclic group with 1-2 heteroatoms, selected independently from O, S and N, which heterocyclic group may bear one or more substituents selected from C₁₋₄alkyl)
or Q¹⁴ⁿ bears a single substituent selected from methylenedioxy and ethylenedioxy).
According to another aspect of the present invention R^{2b} is selected from:
Q^{1b}X¹-
wherein X¹ is as defined hereinbefore and Q^{1b} is selected from one of the following eight groups:
1) Q^{2b} (wherein Q^{2b} is a 5-6membered saturated or partially unsaturated heterocyclic group with 1-2 heteroatoms, selected independently from O, S and N, which heterocyclic group bears at least one substituent selected from C₂₋₅alkenyl, C₂₋₅alkynyl, aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, carbamoylC₁₋₆alkyl, C₁₋₄alkylcarbamoylC₁₋₆alkyl and di(C₁₋₄alkyl)carbamoylC₁₋₆alkyl and which heterocyclic group may optionally bear a further 1 or 2 substituents selected from C₂₋₅alkenyl, C₂₋₅alkynyl, C₁₋₆fluoroalkyl, C₁₋₆alkanoyl, aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl; C₁₋₆fluoroalkanoyl, carbamoyl, C₁₋₄alkylcarbamoyl, di(C₁₋₄alkyl)carbamoyl, carbamoylC₁₋₆alkyl, C₁₋₄alkylcarbamoylC₁₋₆alkyl, di(C₁₋₄alkylcarbamoylC₁₋₆alkyl, C₁₋₆alkylsulphonyl, C₁₋₄fluoroalkylsulphonyl, oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄alkyl, C₁₋₄alkylsulphonylC₁₋₄alkyl, C₁₋₄alkoxycarbonyl, C₁₋₄aminoalkyl, C₁₋₄alkylamino, di(C₁₋₄alkyl)amino, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g} ring D (wherein f is 0 or 1, g is 0 or I and ring D is a 5-6-membered saturated or partially unsaturated heterocyclic group with 1-2 heteroatoms, selected independently from O, S and N, which cyclic group may bear one or more substituents selected from C₁₋₄alkyl),
or Q^{2b} bears a single substituent selected from methylenedioxy and ethylenedioxy); with the proviso that if Q^{1b} is Q^{2b} and X¹ is —O— then Q^{2b} must bear at least one substituent selected from C₂₋₅alkenyl, C₂₋₅alkynyl, C₁₋₄alkoxyC₂₋₆alkanoyl, carbamoylC₁₋₆alkyl, C₁₋₄alkylcarbamoylC₁₋₆alkyl, and di(C₁₋₄alkyl)carbamoylC₁₋₆alkyl and optionally may bear a further 1 or 2 substituents as defined hereinbefore;
2) C₁₋₅alkylW¹Q^{2b} (wherein W¹ and Q^{2b} are as defined hereinbefore);
3) C₁₋₅alkylQ^{2b} (wherein Q^{2b} is as defined hereinbefore);
4) C₂₋₅alkenylQ^{2b} (wherein Q^{2b} is as defined hereinbefore);
5) C₂₋₅alkynylQ^{2b} (wherein Q^{2b} is as defined hereinbefore);
6) C₁₋₄alkylW²C₁₋₄alkylQ^{2b} (wherein W² and Q^{2b} are as defined hereinbefore);
7) C₂₋₅alkenylW²C₁₋₄alkylQ^{2b} (wherein W² and Q^{2b} are as defined hereinbefore); and
8) C₂₋₅alkylW²C₁₋₄alkylQ^{2b} (wherein W² and Q^{2b} are as defined hereinbefore).
According to another aspect of the present invention R^{2b} is selected from:
Q^{1b}X¹-
wherein X¹ is as defined hereinbefore and Q^{1b} is selected from one of the following ten groups:
1) Q^{2b} (wherein Q^{2b} is a 5-6-membered saturated or partially unsaturated heterocyclic group with 1-2 heteroatoms, selected independently from O, S and N, which heterocyclic group bears, at least one substituent selected from aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkamoyl, carbamoylC₁₋₆alkyl, C₁₋₄alkylcarbamoylC₁₋₆alkyl and di(C₁₋₄alkyl)carbamoylC₁₋₆alkyl and which heterocyclic group may optionally bear a further 1 or 2 substituents selected from C₂₋₅alkenyl, C₂₋₅alkynyl, C₁₋₆fluoroalkyl, C₁₋₆alkanoyl, aminoC₂₋₆alkanoyl, C₁₋₄alkylaminoC₂₋₆alkanoyl, di(C₁₋₄alkyl)aminoC₂₋₆alkanoyl, C₄₁₋alkoxyC₁₋₄alkylaminoC₂₋₆alkanoyl, C₁₋₆fluoroalkanoyl, carbamoyl, C₁₋₄alkylcarbamoyl, di(C₁₋₄alkyl)carbamoyl, carbamoylC₁₋₆alkyl, C₁₋₄alkylcarbamoylC₁₋₆alkyl, di(C₁₋₄alkyl)carbamoylC₁₋₆alkyl, C₁₋₆alkylsulphonyl, C₁₋₆fluoroalkylsulphonyl, oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄alkyl, C₁₋₄alkylsulphonylC₁₋₄alkyl, C₁₋₄alkoxycarbonyl, C₁₋₄aminoalkyl, C₁₋₄alkylamino, di(C₁₋₄alkyl)amino, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g} ring D (wherein f is 0 or 1, g is 0 or 1 and ring D is a 5-6-membered saturated or partially unsaturated heterocyclic group with 1-2 heteroatoms, selected independently from O, S and N, which cyclic group may bear one or more substituents selected from C₁₋₄alkyl),
or Q^{2b} bears a single substituent selected from methylenedioxy and ethylenedioxy);
with the proviso that if Q^{1b} is Q^{2b} and X¹ is —O— then Q^{2b} must bear at least one substituent selected from C₁₋₄alkoxyC₁₋₄alkylaminoC₂₋₆alkanoyl, carbamoylC₁₋₆alkyl, C₁₋₄alkylcarbamoylC₁₋₆alkyl, and di(C₁₋₄alkyl)carbamoylC₁₋₆alkyl and optionally may bear a further 1 or 2 substituents as defined hereinbefore;
2) C₁₋₅alkylW¹Q^{2b} (wherein W¹ and Q^{2b} are as defined hereinbefore);
3) C₁₋₅alkylQ^{2b} (wherein Q^{2b} is as defined hereinbefore);
4) C₂₋₅alkenylQ^{2b} (wherein Q^{2b} is as defined hereinbefore);
5) C₂₋₅alkynylQ^{2b} (wherein Q^{2b} is as defined hereinbefore);
6) C₁₋₄alkylW²C₁₋₄alkylQ^{2b} (wherein W² and Q^{2b} are as defined hereinbefore);
7) C₂₋₅alkylW²C₁₋₄alkylQ^{2b} (wherein W² and Q^{2b} are as defined hereinbefore);
8) C₂₋₅alkynylW²C₁₋₄alkylQ^{2b} (wherein W² and Q^{2b} are as defined hereinbefore);
9) C₁₋₄alkylQ^{13b} (C₁₋₄alkyl)ⱼ(W²)ₖQ^{14b} (wherein W^{2b} is as defined hereinbefore, j is 0 or 1, k is 0 or 1, and Q^{13b} and Q^{14b} are each independently selected from hydrogen, C₁₋₃alkyl, cyclopentyl, cyclohexyl and a 5-6-membered saturated or partially unsaturated heterocyclic group with 1-2 heteroatoms, selected independently from O, S and N, which C₁₋₃alkyl group may bear 1 or 2 substituents selected from oxo, hydroxy, halogeno and C₁₋₄alkoxy and which cyclic group may bear 1, 2 or 3 substituents selected from C₂₋₅alkenyl, C₂₋₅alkynyl, C₁₋₆fluoroalkyl, C₁₋₆alkanoyl, aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, C₁₋₆fluoroalkanoyl, carbamoyl, C₁₋₄alkylcarbamoyl, di(C₁₋₄alkyl)carbamoyl, carbamoylC₁₋₆alkyl, C₁₋₄alkylcarbamoylC₁₋₆alkyl, di(C₁₋₄alkyl)carbamoylC₁₋₆alkyl, C₁₋₆alkylsulphonyl, C₁₋₆fluoroalkylsulphonyl, oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄alkyl, C₁₋₄alkylsulphonylC₁₋₄alkyl, C₁₋₄alkoxycarbonyl, C₁₋₄aminoalkyl, C₁₋₄alkylamino, di(C₁₋₄alkyl)amino, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g} ring D (wherein f is 0 or 1, g is 0 or I and ring D is a 5-6-membered saturated or partially unsaturated heterocyclic group with 1-2 heteroatoms, selected independently from O, S and N, which heterocyclic group may bear one or more substituents selected from C₁₋₄alkyl), with the provisos that Q^{13b} cannot be hydrogen and one or both of Q^{13b} and Q^{14b} must be a 5-6membered saturated or partially unsaturated heterocyclic group as defined hereinbefore which heterocyclic group bears at least one substituent selected from aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, carbamoylC₁₋₆alkyl, C₁₋₄alkylcarbamoylC₁₋₆alkyl and di(C₁₋₄alkyl)carbamoylC₁₋₆alkyl and which heterocyclic group optionally bears 1 or 2 further substituents selected from those defined hereinbefore); and
10) C₁₋₄alkylQ¹³-C(O)-C₁₋₄alkylQ¹⁴ⁿ (wherein Q¹³ and Q¹⁴ⁿ are as defined hereinbefore).
According to one aspect of the present invention R^{2b} is selected from:
Q^{1b}X¹-
wherein X¹ is as defined hereinbefore and Q^{1b} is selected from one of the following ten groups:
1) Q^{2b} (wherein Q^{2b} is a 5-6-membered saturated or partially unsaturated heterocyclic group with 1-2 heteroatoms, selected independently from O, S and N, which heterocyclic group bears at least one substituent selected from C₁₋₄alkoxyC₁₋₄alkylaminoC₂₋₆alkanoyl, C₁₋₄alkylcarbamoylC₁₋₆alkyl and di(C₁₋₄alkyl)carbamoylC₁₋₆alkyl and which heterocyclic group may optionally bear a further 1 or 2 substituents selected from C₂₋₅alkenyl, C₂₋₅alkynyl, C₁₋₆fluoroalkyl, C₁₋₆alkanoyl, aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, C₁₋₆fluoroalkanoyl, carbamoyl, C₁₋₄alkylcarbamoyl, di(C₁₋₄alkyl)carbamoyl, carbamoylC₁₋₆alkyl, C₁₋₄alkylcarbamoylC₁₋₆alkyl, di(C₁₋₄alkylcarbamoylC₁₋₆alkyl, C₁₋₆alkylsulphonyl, C₁₋₆fluoroalkylsulphonyl, oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄alkyl, C₁₋₄alkylsulphonylC₁₋₄alkyl, C₁₋₄alkoxycarbonyl, C₁₋₄aminoalkyl, C₁₋₄alkylamino, di(C₁₋₄alkyl)amino, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g} ring D (wherein f is 0 or 1, g is 0 or 1 and ring D is a 5-6-membered saturated or partially unsaturated heterocyclic group with 1-2 heteroatoms, selected independently from O, S and N, which cyclic group may bear one or more substituents selected from C₁₋₄alkyl),
or Q^{2b} bears a single substituent selected from methylenedioxy and ethylenedioxy);
2) C₁₋₅alkylW¹Q^{2b} (wherein W¹ and Q^{2b} are as defined hereinbefore);
3) C₁₋₅alkylQ^{2b} (wherein Q^{2b} is as defined hereinbefore);
4) C₂₋₅alkenylQ^{2b} (wherein Q^{2b} is as defined hereinbefore);
5) C₂₋₅alkynylQ^{2b} (wherein Q^{2b} is as defined hereinbefore);
6) C₁₋₄alkylW²C₁₋₄alkylQ^{2b} (wherein W² and Q^{2b} are as defined hereinbefore);
7) C₂₋₅alkenylQ^{2b} (wherein W² and Q^{2b} are as defined hereinbefore);
8) C₂₋₅alkynylW²C₁₋₄-alkylQ^{2b} (wherein W² and Q^{2b} are as defined hereinbefore);
9) C₁₋₄alkylQ^{13b}(C₁₋₄alkyl)ⱼ(W²)ₖQ^{14b} (wherein W² is as defined hereinbefore, j is 0 or 1, k is 0 or 1, and Q^{13b} and Q^{14b} are each independently selected from hydrogen, C₁₋₃alkyl, cyclopentyl, cyclohexyl and a 5-6-membered saturated or partially unsaturated heterocyclic group with 1-2 heteroatoms, selected independently from O, S and N, which C₁₋₃alkyl group may bear 1 or 2 substituents selected from oxo, hydroxy, halogeno and C₁₋₄alkoxy and which cyclic group may bear 1, 2 or 3 substituents selected from C₂₋₅alkenyl, C₂₋₅alkynyl, C₁₋₆fluoroalkyl, C₁₋₆alkanoyl, aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, C₁₋₄fluoroalkanoyl, carbamoyl, C₁₋₄alkylcarbamoyl, di(C₁₋₄alkyl)carbamoyl, carbamoylC₁₋₆alkyl, C₁₋₄alkycarbamoylC₁₋₆alkyl, di(C₁₋₄alkyl)carbamoylC₁₋₆alkyl, C₁₋₆alkylsulphonyl, C₁₋₆fluoroalkylsulphonyl, oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄alkyl, C₁₋₄alkylsulphonylC₁₋₄alkyl, C₁₋₄alkoxycarbonyl, C₁₋₄aminoalkyl, C₁₋₄alkylamino, di(C₁₋₄alkyl)amino, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g} ring D (wherein f is 0 or 1, g is 0 or 1 and ring D is a 5-6-membered saturated or partially unsaturated heterocyclic group with 1-2 heteroatoms, selected independently from O, S and N, which heterocyclic group may bear one or more substituents selected from C₁₋₄alkyl), with the provisos that Q^{13b} cannot be hydrogen and one or both of Q^{13b} and Q^{14b} must be a 5-6-membered saturated or partially unsaturated heterocyclic group as defined hereinbefore which heterocyclic group bears at least one substituent selected from C₁₋₄alkoxyC₁₋₄alkylaminoC₂₋₆alkanoyl, C₁₋₄alkylcarbamoylC₁₋₄alkyl and di(C₁₋₄alkyl)carbamoylC₁₋₆alkyl and which heterocyclic group optionally bears 1 or 2 further substituents selected from those defined hereinbefore); and
10) C₁₋₄alkylQ^{13b}-C(O)-C₁₋₄alkylQ^{14b} (wherein Q^{13b} and Q^{14b} are as defined hereinbefore and with the provisos that Q^{13b} cannot be hydrogen and one or both of Q^{13b} and Q^{14b} must be a 5-6-membered saturated or partially unsaturated heterocyclic group as defined hereinbefore which heterocyclic group bears at least one substituent selected from C₁₋₄alkoxyC₁₋₄alkylaminoC₂₋₆alkanoyl, C₁₋₄alkylcarbamoylC₁₋₆alkyl and di(C₁₄alkyl)carbamoylC₁₋₆alkyl and which heterocyclic group optionally bears 1 or 2 further substituents selected from those defined hereinbefore).

Particular compounds of the present invention include:
4-(4-bromo-2-fluoroanilino)-7-({1-[(N,N-dimethylamino)acetyl]piperidin-4-yl}methoxy)-6-methoxyquinazoline,
4-(4chloro-2-fluoroanilino)-7-({1-[(N,N-dimethylamino)acetyl]piperidin-4-yl}methoxy)-6-methoxyquinazoline,
4-(4-chloro-2-fluoroanilino)-6-methoxy-7-{[1-pyrrolidin-1-ylacetyl)piperidin-4-yl]methoxy}quinazoline,
4-(4-chloro-2-fluoroanilino)-6-methoxy-7-{[1-(piperidin-1-ylacetyl)piperidin-4-yl]methoxy}quinazoline,
4-(4-chloro-2-fluoroanilino)-6-methoxy-7-{[1-(morpholin-4-ylacetyl)piperidin-4-yl]methoxy}quinazoline,
4-(4-chloro-2-fluoroanilino)-6-methoxy-7-({1-[(3aR,6aS)-tetrahydro-5H-[1,3]dioxolo[4,5-c]pyrrol-5-ylacetyl]piperidin-4-yl}methoxy)quinazoline,
7-({1-[(4-acetylpiperazin-1-yl)acetyl]piperidinylmethoxy)-4-yl}methoxy)-(4-chloro-2-fluoroanilino)-6-methoxyquinazoline,
(3S)-4-(4-chloro-2-fluoroanilio)-7-({1-[(3-hydroxypyrrolidin-1-yl)acetyl]piperidin-4-yl}methoxy)-6-methoxyquinazoline,
4-(4-chloro-2-fluoroanilino)-6-methoxy-7-[(1-{[N-(2methoxyethyl)amino]acetyl}piperidin-4yl)methoxy]quinazoline,
4-(4-chloro-2-fluoroanilino)-6-methoxy-7-({1-[(N-methylamino)acetyl]piperidin-4-yl}methoxy)quinazoline,
4-(4-chloro-2-fluoroanilino)-7-({1-[(3,3-difluoropyrrolidin-1-yl)acetylopiperidin-4-yl}methoxy)-6-methoxyquinazoline,
4-(4-chloro-2-fluoroanilino)-7-(2-{1-[(N,N-dimethylamino)acetyl]piperidin-4-yl}ethoxy)-6-methoxyquinazoline,
4-(4-bromo-2-fluoroanilino)-7-(2-{1-[(N,N-dimethylamino)acetyl]piperidin-4-yl}ethoxy)-6-methoxyquinazoline,
4-(4-chloro-2-fluoroanilino)-7-({{3R)-1-[(N,N-dimethylamino)acetyl]piperidin-3-yl}methoxy)6-methoxyquinazoline,
4(Chloro-2-fluoroanilino)-7-({(3Si-[(N,N-dimethylamino)acetyl]piperidin-3-yI}methoxy)6-methoxyquinazoline,
4-(4bromo-2-fluoroanilino-6-methoxy-7- {3-[(3aR,6aS)-tetrahydro-5H-[1,3]dioxolo[4,S-c]pyrrol-5-yl]propoxy} quinazoline,
4-(4-bromo-2-fluoroanilino)-6-methoxy-7- {2-[(3aR,6-tetrahydro-5H-[1,3]dioxolo[4,5-c]pyrrol-5-yl]ethoxy}quinazoline,
   and salts thereof.

For the avoidance of doubt it is to be understood that where in this specification a group is qualified by ‘hereinbefore defined’ or ‘defined hereinbefore’ the said group encompasses the first occurring and broadest definition as well as each and all of the preferred definitions for that group.

In this specification unless stated otherwise the term “alkyl” includes both straight and branched chain alkyl groups but references to individual alkyl groups such as “propyl” are specific for the straight chain version only. An analogous convention applies to other generic terms. Unless otherwise stated the term “alkyl” advantageously refers to chains with 1-6 carbon atoms, preferably 14 carbon atoms. The term “alkoxy” as used herein, unless stated otherwise includes “alkyl” —O— groups in which “alkyl” is as hereinbefore defined. The term “aryl” as used herein unless stated otherwise includes reference to a C₆₋₁₀ aryl group which may, if desired, carry one or more substituents selected from halogeno, alkyl alkoxy, nitro, trifluoromethyl and cyano, (wherein alkyl and alkoxy are as hereinbefore defined). The term “aryloxy” as used herein unless otherwise stated includes “aryl” —O— groups in which “aryl” is as hereinbefore defined. The term “silphonyloxy” as used herein refers to alkylsulphonyloxy and arylsulphonyloxy groups in which “alkyl” and “aryl” are as hereinbefore defined. The term “alkanoyl” as used herein unless otherwise stated includes formyl and alkylC═O groups in which “alkyl” is as defined hereinbefore, for example C₂alkanoyl is ethanoyl and refers to CH₃C═O, C₁alkanoyl is formyl and refers to CHO. Butanoyl refers to CH₃—CH₂—CH₂—C(O), isobutyryl refers to (CH₃)₂.CH—C(O). In this specification unless stated otherwise the term “alkenyl” includes both straight and branched chain alkenyl groups but references to individual alkenyl groups such as 2-butenyl are specific for the straight chain version only. Unless otherwise stated the term “alkenyl” advantageously refers to chains with 2-5 carbon atoms, preferably 3-4 carbon atoms. In this specification unless stated otherwise the term “alkynyl” includes both straight and branched chain alkynyl groups but references to individual alkynyl groups such as 2-butynyl are specific for the straight chain version only. Unless otherwise stated the term “alkynyl” advantageously refers to chains with 2-5 carbon atoms, preferably 3-4 carbon atoms. Unless stated otherwise the term “haloalkyl” refers to an alkyl group as defined hereinbefore which bears one or more halogeno groups, such as for example trifluoromethyl.

Within the present invention it is to be understood that a compound of the formula I or a salt thereof may exhibit the phenomenon of tautomerism and that the formulae drawings within this specification can represent only one of the possible tautomeric forms. It is to be understood that the invention encompasses any tautomeric form which inhibits VEGF receptor tyrosine kinase activity and is not to be limited merely to any one tautomeric form utilised within the formulae drawings. The formulae drawings within this specification can represent only one of the possible tautomeric forms and it is to be understood that the specification encompasses all possible tautomeric forms of the compounds drawn not just those forms which it has been possible to show graphically herein.

It will be appreciated that compounds of the formula I or a salt thereof may possess an asymmetric carbon atom. Such an asymmetric carbon atom is also involved in the tautomerism described above, and it is to be understood that the present invention encompasses any chiral form (including both pure enantiomers, scalemic and racemic mixtures) as well as any tautomeric form which inhibits VEGF receptor tyrosine kinase activity, and is not to be limited merely to any one tautomeric form or chiral form utilised within the formulae drawings. It is to be understood that the invention encompasses all optical and diastereomers which inhibit VEGF receptor tyrosine kinase activity. It is ether to be understood that in the names of chiral compounds (R,S) denotes any scalemic or racemic mixture while (R) and (S) denote the enantiomers. In the absence of (R,S), (R) or (S) in the name it is to be understood that the name refers to any scalemic or racemic mixture, wherein a scalemic mixture contains R and S enantiomers in any relative proportions and a racemic mixture contains R and S enantiomers in the ration 50:50.

It is also to be understood that certain compounds of the formula I and salts thereof can exist in solvated as well as unsolvated forms such as, for example, hydrated forms. It is to be understood that the invention encompasses all such solvated forms which inhibit VEGF receptor tyrosine kinase activity.

For the avoidance of any doubt, it is to be understood that when X¹ is —NR⁴— it is the nitrogen atom bearing the R⁴ group which is linked to the quinazoline ring and to Q¹ and an analogous convention applies to similar groups. When W¹ is, for example, a group of formula —NQ³C(O)—, it is the nitrogen atom bearing the Q³ group, which is attached to the C₁₋₅alkyl group and the carbonyl (C(O)) group is attached to Q², whereas when W¹ is, for example, a group of formula —C(O)NQ⁴-, it is the carbonyl group which is attached to the C₁₋₅alkyl) group and the nitrogen atom bearing the Q⁴ group is attached to Q². A similar convention applies to the other two atom W¹ linking groups such as —NQ⁶SO₂— and —SO₂NQ⁵-. An analogous convention applies to other groups. It is further to be understood that when X¹ represents —NR⁴— and R⁴ is C₁₋₃alkoxyC₂₋₃alkyl it is the C₂-₃alkyl moiety which is linked to the nitrogen atom of X¹ and an analogous convention applies to other groups.

For the avoidance of any doubt, it is to be understood that in a compound of the formula I when Q¹ is, for example, a group of formula C₁₋₄alkylIW²C₁₋₄alkylQ², it is the terminal C₁₋₄alkyl moiety which is linked to X¹, which is in turn linked to the quinazoline ring, similarly when Q¹ is, for example, a group of formula C₂₋₅alkenylQ² it is the C₂₋₅alkenyl moiety which is linked to X¹ and an analogous convention applies to other groups. When Q¹ is a group 1-Q²prop-1-en-3-yl it is the first carbon to which the group Q² is attached and it is third carbon which is linked to X¹ and an analogous convention applies to other groups.

For the avoidance of any doubt, it is to be understood that in a compound of the formula I when Q¹ is, for example, Q² and Q² is a pyrrolidinyl ring which bears a group —(—O—)_{f}(C₁₋₄alkyl)_{g} ring, it is the —O— or C₁₋₄alkyl which is linked to the pyrrolidinyl ring, unless f and g are both 0 when it is ring D which is linked to the pyrrolidinyl ring and an analogous convention applies to other groups.

For the avoidance of any doubt, it is to be understood that when Q² carries a C₁₋₄aminoalkyl substituent it is the C₁₋₄alkyl moiety which is attached to Q² whereas when Q² carries a C₁₋₄alkylamino substituent it is the amino moiety which is attached to Q² and an analogous convention applies to other groups.

For the avoidance of any doubt, it is to be understood that when Q² carries a C₁₋₄alkoxyC₁₋₄alkyl substituent it is the C₁₋₄alkyl moiety which is attached to Q² and an analogous convention applies to other groups.

For the avoidance of any doubt, it is to be understood that when R² is a group Q¹⁵W³ it is the W³ group which is linked to the quinazoline ring.

For the avoidance of any doubt, it is to be understood that when R² is a group Q²¹W⁴C₁₋₅alkylX¹ it is the X¹ group which is linked to the quinazoline ring.

For the avoidance of any doubt it is to be understood that when the phrase “a 5-6 membered saturated or partially unsaturated heterocyclic group” is used herein for the values of, for example, Q², ring D, Q¹³, Q¹⁴ and Q¹⁴ⁿ it does not include the value pyrridone. Thus Q², ring D, Q¹³, Q¹⁴ and Q¹⁴ⁿ cannot be pyridone.

Compounds of formula I may be administered in the form of a prodrug which is broken down in the human or animal body to give a compound of the formula I. Examples of prodrugs include in vivo hydrolysable esters of a compound of the formula I.

Various forms of prodrugs are known in the art. For examples of such prodrug derivatives see:
a) Design of Prodrugs, edited by H. Bundgaard, (Elsevier, 1985) and Methods in Enzymology, Vol. 42, p. 309-396, edited by K. Widder, et aL. (Academic Press, 1985);
b) A Textbook of Drug Design and Development, edited by Krogsgaard-Larsen and H. Bundgaard, Chapter 5 “Design and Application of Prodrugs”, by H. Bundgaard p. 113-191 (1991);
c) H. Bundgaard, Advanced Drug Delivery Reviews, 8, 1-38 (1992);
d) H. Bundgaard, et al., Journal of Pharmaceutical Sciences, 77 285 (1988); and
e) N. Kakeya, et al., Chem Pharm Bull, 32, 692 (1984).

An in vivo hydrolysable ester of a compound of formula I containing a hydroxy group includes inorganic esters such as phosphate esters (including phosphoramidic cyclic esters) and a-acyloxyalkyl ethers and related compounds which as a result of the in vivo hydrolysis of the ester breakdown to give the parent hydroxy group/s. Examples of a-acyloxyalkyl ethers include acetoxymethoxy and 2,2-dimethylpropionyloxy-methoxy. A selection of in vivo hydrolysable ester-forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. Examples of substituents on benzoyl include morpholino and piperazine linked from a ring nitrogen atom via a methylene group to the 3- or 4-position of the benzoyl ring.

The present invention relates to the compounds of formula I as hereinbefore defined as well as to the salts thereof. Salts for use in pharmaceutical compositions will be pharmaceutically acceptable salts, but other salts may be useful in the production of the compounds of formula I and their pharmaceutically acceptable salts. Pharmaceutically acceptable salts of the invention may, for example, include acid addition salts of the compounds of formula I as hereinbefore defined which are sufficiently basic to form such salts. Such acid addition salts include for example salts with inorganic or organic acids affording pharmaceutically acceptable anions such as with hydrogen halides (especially hydrochloric or hydrobromic acid of which hydrochloric acid is particularly preferred) or with sulphuric or phosphoric acid, or with trifluoroacetic, citric or maleic acid. In addition where the compounds of formula I are sufficiently acidic, pharmaceutically acceptable salts may be formed with an inorganic or organic base which affords a pharmaceutically acceptable cation. Such salts with inorganic or organic bases include for example an alkali metal salt, such as a sodium or potassium salt, an alkaline earth metal salt such as a calcium or magnesium salt, an ammonium salt or for example a salt with methylamine, dimethylamine, trimethylamine, piperidine, morpholine or tris-(2-hydroxyethyl)amine.

A compound of the formula I, or salt thereof, and other compounds of the invention (as herein defined) may be prepared by any process known to be applicable to the preparation of chemically-related compounds. Such processes include, for example, those illustrated in International Patent Applications Publication Numbers WO 98/13354 and WO 01/32651, WO 97/22596, WO 97/30035, WO 97/32856 and in European Patent Applications Publication Nos. 0520722, 0566226, 0602851 and 0635498. Such processes also include, for example, solid phase synthesis. Such processes, are provided as a further feature of the invention and are as described hereinafter. Necessary starting materials may be obtained by standard procedures of organic chemistry. The preparation of such starting materials is described within the accompanying non-limiting Examples. Alternatively necessary starting materials are obtainable by analogous procedures to those illustrated which are within the ordinary skill of an organic chemist.

Thus the following processes (a) to (e) and (i) to (iv) constitute further features of the present invention

Synthesis of Compounds of Formula I

(a) Compounds of the formula I and salts thereof may be prepared by the reaction of a compound of the formula II: 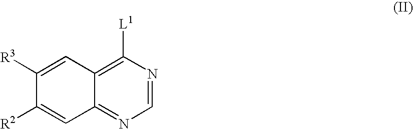
(wherein R² and R³ are as defined hereinbefore and L¹ is a displaceable moiety), with a compound of the formula III: 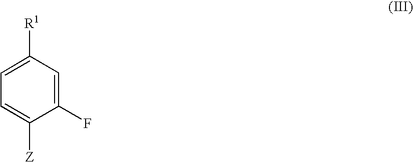
(wherein R¹ and Z are as defined hereinbefore) whereby to obtain compounds of the formula I and salts thereof. A convenient displaceable moiety L¹ is, for example, a halogeno, alkoxy (preferably C₁₋₄alkoxy), aryloxy or sulphonyloxy group, for example a chloro, bromo, methoxy, phenoxy, methanesulphonyloxy or toluene sulphonyloxy group.

The reaction is advantageously effected in the presence of either an acid or a base. Such an acid is, for example, an anhydrous inorganic acid such as hydrogen chloride. Such a base is, for example, an organic amine base such as, for example, pyridine, 2,6-lutidine, collidine, 4-dimethylaminopyridine, triethylamine, morpholine, N-methylnorpholine or diazabicyclo[5.4.0]undec-7-ene, or for example, an alkali metal or alkaline earth metal carbonate or hydroxide, for example sodium carbonate, potassium carbonate, calcium carbonate, sodium hydroxide or potassium hydroxide. Alternatively such a base is, for example, an alkali metal hydride, for example sodium hydride, or an alkali metal or alkaline earth metal amide, for example sodium amide or sodium bis(trimethylsilyl)amide. The reaction is preferably effected in the presence of an inert solvent or diluent, for example an alkanol or ester such as methanol, ethanol, 2-propanol or ethyl acetate, a halogenated solvent such as methylene chloride, trichloromethane or carbon tetrachloride, an ether such as tetrahydrofuran or 1,4-dioxan, an aromatic hydrocarbon solvent such as toluene, or a dipolar aprotic solvent such as N,N-dimethylformamide, N,N-dimethylacetamide, N-methylpyrrolidin-2-one or dimethylsulphoxide. The reaction is conveniently effected at a temperature in thee range, for example, 10 to 150° C., preferably in the range 20 to 80° C.

The compound of the invention may be obtained from this process in the form of the free base or alternatively it may be obtained in the form of a salt with the acid of the formula H-L¹ wherein L¹ has the meaning defined hereinbefore. When it is desired to obtain the free base from the salt, the salt may be treated with a base as defined hereinbefore using a conventional procedure.

When it is desired to obtain the acid salt, the free base may be treated with an acid such as a hydrogen halide, for example hydrogen chloride, sulphuric acid, a sulphonic acid, for example methane sulphonic acid, or a carboxylic acid, for example acetic or citric acid, using a conventional procedure.

(b) Compounds of the formula I and salts thereof may be prepared by the reaction, conveniently in the presence of a base as defined hereinbefore, of a compound of the formula IV: 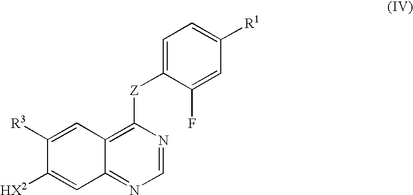
(wherein Z, R¹ and R³ are as hereinbefore defined) with a compound of formula V:
R⁵-L¹ (V)
(wherein R⁵ is Q¹, Q¹⁵ or Q²¹W⁴C₁₋₅alkyl, X² is X¹ or W³ and L¹ is as hereinbefore defined); L¹ is a displaceable moiety for example a halogeno or sulphonyloxy group such as a bromo or methanesulphonyloxy group. Conveniently L¹ is a group O-⁺P(Y)3 (wherein Y is butyl or phenyl) and in such cases the compound of formula V is conveniently formed in situ. The reaction is preferably effected in the presence of a base (as defined hereinbefore in process (a)) and advantageously in the presence of an inert solvent or diluent (as defined hereinbefore in process (a)), advantageously at a temperature in the range, for example 10 to 150° C., conveniently at about 50° C.

(c) Compounds of the formula I and salts thereof may be prepared by the reaction of a compound of the formula VI: 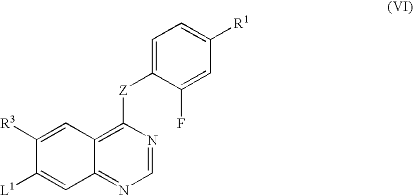
with a compound of the formula VIIa-c:
Q¹-X¹—H (VIIa)
Q¹⁵-W³—H (VIIb)
Q²¹-W⁴-C₁₋₅alkyl-X¹—H (VIIc)
(wherein L¹, R¹, R³, Z, Q¹, Q¹⁵, Q²¹W³, W⁴ and X¹ are all as hereinbefore defined). The reaction may conveniently be effected in the presence of a base (as defined hereinbefore in process (a)) and advantageously in the presence of an inert solvent or diluent (as defined hereinbefore in process (a)), advantageously at a temperature in the range, for example 10 to 150° C., conveniently at about 100°0 C.

(d) Compounds of the formula I and salts thereof may be prepared by the deprotection of a compound of the formula VIII: 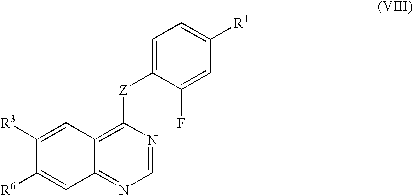
wherein R¹, R³ and Z are all as hereinbefore defined, and R⁶ represents a protected ² group wherein R² is as defined hereinbefore but additionally bears one or more protecting groups P². The choice of protecting group P² is within the standard knowledge of an organic chemist, for example those included in standard texts such as “Protective Groups in Organic Synthesis” T. W. Greene and R. G. M. Wuts, 2nd Ed. Wiley 1991. Preferably P² is a protecting group such as a carbamate (alkoxycarbonyl) (such as, for example, tert-butoxycarbonyl, tert-amyloxycarbonyl, cyclobutoxycarbonyl, propoxycarbonyl, methoxycarbonyl, ethoxycarbonyl, isopropoxycarbonyl allyloxycarbonyl or benzyloxycarbonyl). More preferably p is tert-butoxycarbonyl. The reaction is preferably effected in the presence of an acid. Such an acid is, for example, an inorganic acid such as hydrogen chloride, hydrogen bromide or an organic acid such as trifluoroacetic acid, trifluoromethane sulphonic acid. The reaction may be effected in the presence of an inert solvent such as methylene chloride, trichloromethane and in the presence of a trace of water. The reaction is conveniently effected at a temperature in the range, for example, 10-100° C., preferably in the range 20-80° C.
(e) Compounds of the formula I and salts thereof may be prepared by the addition of a substituent to a compound of the formula IX: 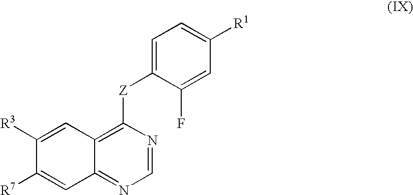
wherein R¹, R³ and Z are all as hereinbefore defined, and R⁷ represents an R² group which has yet to be substituted with its final substituent.

For example where R² contains a heterocyclic ring with a substituent it is possible to add the substituent after process (a) above using standard procedures of organic chemistry. Thus for example a compound of formula II as defined hereinbefore but wherein R² contains an unsubstituted heterocyclic ring may be reacted with a compound of formula III as defined hereinbefore to give an intermediate compound in which R² contains an unsubstituted heterocyclic ring. The intermediate compound can then be substituted on the heterocyclic ring in R² using standard organic chemistry techniques to give a final compound of formula I.

Synthesis of Intermediates

(i) The compounds of formula III and salts thereof in which L¹ is halogeno may for example be prepared by halogenating a compound of the formula X: 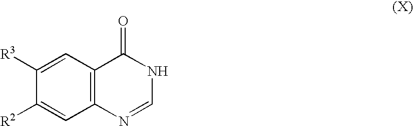
(wherein R² and R³ are as hereinbefore defined).

Convenient halogenating agents include inorganic acid halides, for example thionyl chloride, phosphorus(III)chloride, phosphorus(V)oxychloride and phosphorus(V)chloride. The halogenation reaction is conveniently effected in the presence of an inert solvent or diluent such as for example a halogenated solvent such as methylene chloride, trichloromethane or carbon tetrachloride, or an aromatic hydrocarbon solvent such as benzene or toluene. The reaction is conveniently effected at a temperature in the range, for example 10 to 150° C., preferably in the range 40 to 100° C.

The compounds of formula X and salts thereof may for example be prepared by reacting a compound of the formula XI: 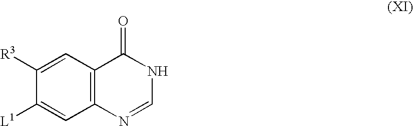
(wherein R³ and L¹ are as hereinbefore defined) with a compound of the formula VII as hereinbefore defined. The reaction may conveniently be effected in the presence of a base (as defined hereinbefore in process (a)) and advantageously in the presence of an inert solvent or diluent (as defined hereinbefore in process (a)), advantageously at a temperature in the range, for example 10 to 150° C., conveniently at about 100° C.

The compounds of formula X and salts thereof may also be prepared by cyclising a compound of the formula XII: 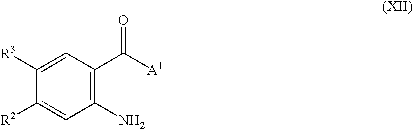
(wherein R² and R³, are as hereinbefore defined, and A¹ is an hydroxy, alkoxy (preferably C₁₋₄alkoxy) or amino group) whereby to form a compound of formula X or salt thereof The cyclisation may be effected by reacting a compound of the formula XII, where A¹ is an hydroxy or alkoxy group, with formamide or an equivalent thereof effective to cause cyclisation whereby a compound of formula X or salt thereof is obtained, such as [3-(dimethylamino)-2-azaprop-2-enylidene]dimethylammonium chloride. The cyclisation is conveniently effected in the presence of formamide as solvent or in the presence of an inert solvent or diluent such as an ether for example 1,4-dioxan. The cyclisation is conveniently effected at an elevated temperature, preferably in the range 80-to 200° C. The compounds of formula X may also be prepared by cyclising a compound of the formula XII, where A¹ is an amino group, with formic acid or an equivalent thereof effective to cause cyclisation whereby a compound of formula X or salt thereof is obtained. Equivalents of formic acid effective to cause cyclisation include for example a tri-C₁₋₄alkoxymethane, for example triethoxymethane and trimethoxymethane. The cyclisation is conveniently effected in the presence of a catalytic amount of an anhydrous acid, such as a sulphonic acid for example p-toluenesulphoric acid, and in the presence of an inert solvent or diluent such as for example a halogenated solvent such as methylene chloride, trichloromethane or carbon tetrachloride, an ether such as diethyl ether or tetrahydrofuran, or an aromatic hydrocarbon solvent such as toluene. The cyclisation is conveniently effected at a temperature in the range, for example 10 to 100° C, preferably in he range 20 to 50° C.

Compounds of formula XII and, salts thereof may for example be prepared by the reuction of the nitro group in a compound of the formula XIII: 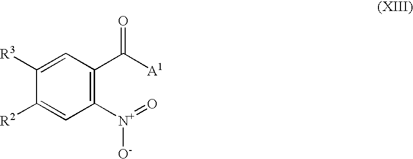
(wherein R², R³ and A¹ are as hereinbefore defined) to yield a compound of formula XII as hereinbefore defined. The reduction of the nitro group may conveniently be effected by any of the procedures known for such a transformation. The reduction may be carried out, for example, by the hydrogenation of a solution of the nitro compound in the presence of an inert solvent or diluent as defined hereinbefore in the presence of a metal effective to catalyse hydrogenation reactions such as palladium or platinum. A further reducing agent is, for example, an activated metal such as activated iron (produced for example by washing iron powder with a dilute solution of an acid such as hydrochloric acid). Thus, for example, the reduction may be effected by heating the nitro compound and the activated metal in the presence of a solvent or diluent such as a mixture of water and alcohol, for example methanol or ethanol, to a temperature in the range, for example 50 to 150° C., conveniently at about 70° C.

Compounds of the formula XIII and salts thereof may for example be prepared by the reaction of a compound of the formula XIV: 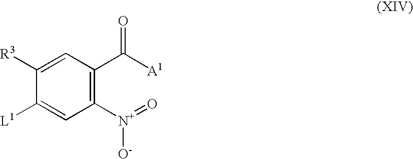
(wherein R³, L¹ and A¹ are as hereinbefore defined) with a compound of the formula VII as hereinbefore defined to give a compound of the formula II. The reaction of the compounds of formulae XIV and VII is conveniently effected under conditions as described for process (c) hereinbefore.

Compounds of formula XIII and salts thereof may for example also be prepared by the reaction of a compound of the formula XV: 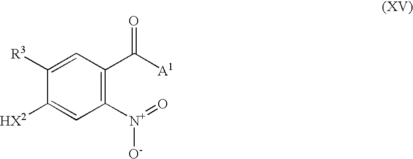
(wherein R³, X² and A¹ are as hereinbefore defined) with a compound of the formula V as hereinbefore defined to yield a compound of formula XIII as hereinbefore defined. The reaction of the compounds of formulae XV and V is conveniently effected under conditions as described for process (b) hereinbefore.

The compounds of formula II and salts thereof may also be prepared for example by reacting a compound of the formula XVI: 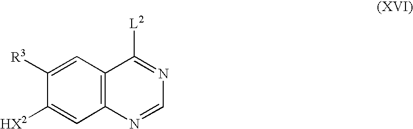
(wherein R³ and X² are as hereinbefore defined and L² represents a displaceable protecting moiety) with a compound of the formula V as hereinbefore defined, whereby to obtain a compound of formula II in which L¹ is represented by L².

A compound of formula XVI is conveniently used in which L² represents a phenoxy group which may if desired carry up to 5 substients, preferably up to 2 substituents, selected from halogeno, nitro and cyano. The reaction may be conveniently effected under conditions as described for process (b) hereinbefore.

The compounds of formula XVI and salts thereof as hereinbefore defined may for example be prepared by deprotecting a compound of the formula XVII: 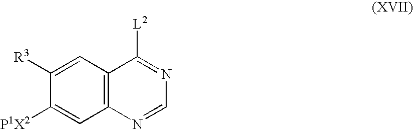
(wherein R³, X² and L² are as hereinbefore defined and P¹ represents a phenolic hydroxy protecting group). The choice of phenolic hydroxy protecting group P¹ is within the standard knowledge of an organic chemist, for example those included in standard texts such as “Protective Groups in Organic Synthesis” T. W. Greene and R. G. M. Wuts, 2nd Ed. Wiley 1991, including ethers (for example, methyl, methoxymethyl, allyl and benzyl and benzyl substituted with up to two substituents selected from C₁₋₄alkoxy and nitro), silyl ethers (for example, t-butyldiphenylsilyl and t-butyldimethylsilyl), esters (for example, acetate and benzoate) and carbonates (for example, methyl and benzyl and benzyl substituted with up to two substituents selected from C₁₋₄alkoxy and nitro). Deprotection may be effected by techniques well known in the literature, for example where P¹ represents a benzyl group deprotection may be effected by hydrogenolysis or by treatment. with trifluoroacetic acid.

The removal of such a phenolic hydroxy protecting group may be effected by any of the procedures known for such a transformation, including those reaction conditions indicated in standard texts such as that indicated hereinbefore, or by a related procedure. The reaction conditions preferably being such that the hydroxy derivative is produced without unwanted reactions at other sites within the starting or product compounds. For examples where the protecting group P¹ is acetate, the transformation may conveniently be effected by treatment of the quinazoline derivative with a base as defined hereinbefore and including ammonia, and its mono and di-alkylated derivatives, preferably in the presence of a protic solvent or co-solvent such as water or an alcohol, for example methanol or ethanol. Such a reaction can be effected in the presence of an additional inert solvent or diluent as defined hereinbefore and at a temperature in the range 0 to 50° C., conveniently at about 20° C.

One compound of formula II may if desired be converted into another compound of formula II in which the moiety L¹ is different. Thus for example a compound of formula II in which L¹ is other than halogeno, for example optionally substituted phenoxy, may be converted to a compound of formula II in which L¹ is halogeno by hydrolysis of a compound of formula II (in which L¹ is other than halogeno) to yield a compound of formula X as hereinbefore defined, followed by introduction of halide to the compound of formula X, thus obtained as hereinbefore defined, to yield a compound of formula II in which L¹ represents halogeno.
(ii) Compounds of the formula IV as hereinbefore defined and salts thereof may be made by deprotecting the compound of formula XVIII: 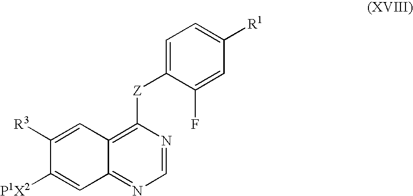
(wherein R¹, R³, P¹, X² and Z are as herrinbefore defined) by a process for example as described in (i) above.

Compounds of the formula XVIII and salts thereof may be made by reacting compounds of the formulae XVII and III as hereinbefore defined, under the conditions described in (a) hereinbefore, to give a compound of the formula XVIII or salt thereof.

(iii) Compounds of the formula VI and salts thereof as hereinbefore defined may be made by reacting a compound of the formula XIX: 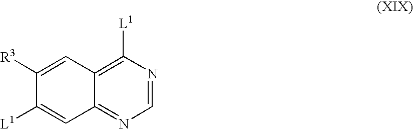
(wherein R³ and L¹ are as hereinbefore defined, and L¹ in the 4 and 7-positions may be the same or different) with a compound of formual III as hereinbefore defined, the reaction for example being effected by a process as described in (a) above.

(iv) A compound of the formula VIII may be prepared by the reaction of a compound of the formula IV as defined hereinbefore with a compound of the formula XX:
R⁶-L¹ (XX)
wherein R⁶ and L¹ are as defined hereinbefore under the conditions described in (b) hereinbefore to give a compound of the formula VIII or salt thereof. The reaction is preferably effected in the presence of a base (as defined hereinbefore in process (a)) and advantageously in the presence of an inert solvent or diluent (as defined hereinbefore in process (a)), advantageously at a temperature in the range, for example 10 to 150° C., conveniently in the range 20-50° C.

When a pharmaceutically acceptable salt of a compound of the formula I is required, it may be obtained, for example, by reaction of said compound with, for example, an acid using a conventional procedure, the acid having a pharmaceutically acceptable anion.

Certain of the intermediates herein are novel and these are presented as a further aspect of the present invention.

The identification of compounds which potently inhibit the tyrosine kinase activity associated with the VEGF receptors such as Flt and/or KDR, which inhibit the tyrosine kinase activity associated with the EGF receptor and which are inactive or only weakly active in the hERG assay, is desirable and is the subject of the present invention.

These properties may be assessed, for example, using one or more of the procedures set out below:

(a) In Vitro Receptor Tyrosine Kinase Inhibition Test

This assay determines the ability of a test compound to inhibit tyrosine kinase activity. DNA encoding VEGF or epidermal growth factor (EGF) receptor cytoplasmic domains may be obtained by total gene synthesis (Edwards M, International Biotechnology Lab 5(3), 19-25, 1987) or by cloning. These may then be expressed in a suitable expression system to obtain polypeptide with tyrosine kinase activity. For example VEGF and EGF receptor cytoplasmic domains, which were obtained by expression of recombinant protein in insect cells, were found to display intrinsic tyrosine kinase activity. In the case of the VEGF receptor Flt (Genbank accession number X51602), a 1.7 kb DNA fragment encoding most of the cytoplasmic domain, commencing with methionine 783 and including the termination codon, described by Shibuya et al (Oncogene, 1990, 5: 519-524), was isolated from cDNA and cloned into a baculoviris transplacement vector (for example pAcYM1 (see The Baculovirus Expression System: A Laboratory Guide, L. A. King and R. D. Possee, Chapman and Hall, 1992) or pAc360 or pBlueBacHis (available from Invitrogen Corporation)). This recombinant construct was co-transfected into insect cells (for example Spodoptera frugiperda 21(Sf21)) with viral DNA (eg Pharmingen BaculoGold) to prepare recombinant baculovirus. (Details of the methods for the assembly of recombinant DNA molecules and the preparation and use of recombinant baculovirus can be found in standard texts for example Sambrook et al, 1989, Molecular cloning—A Laboratory Manual, 2nd. edition, Cold. Spring Harbour Laboratory Press and O'Reilly et al, 1992, Baculovirus Expression Vectors—A Laboratory Manual, W. H. Freeman and Co, New York). For other tyrosine kinases for use in assays, cytoplasmic fragments starting from methionine 806 (KDR, Genbafik accession number L04947) and methionine 668 (EGF receptor, Genbank accession number X00588) may be cloned and expressed in a similar manner.

For expression of cFlt tyrosine kinase activity, Sf21 cells were infected with plaque-pure cFlt recombinant virus at a multiplicity of infection of 3 and harvested 48 hours later. Harvested cells were washed with ice cold phosphate-buffered saline solution (PBS) (10 mM sodium phosphate pH7.4, 138 mM sodium chloride, 2.7 mM potassium chloride) then resuspended in ice cold HNTG/PMSF (20 mM Hepes pH7.5, 150 mM sodium chloride, 10% v/v glycerol, 1% v/v Triton X100, 1.5 mM magnesium chloride, 1 mM ethylene glycol-bis(βaminoethyl ether) N,N,N′,N′-tetraacetic acid (EGTA), 1 mM PMSF (phenylmethylsulphonyl fluoride); the PMSF is added just before use from a freshly-prepared 100 mM solution in methanol) using 1 ml HNTG/PMSF per 10 million cells. The suspension was centrifuged for 10 minutes at 13,000 rpm at 4° C., the supematat (enzyme stock) was removed and stored in aliquots at −70° C. Each new batch of stock enme was titrated in the assay by dilution with enzyme diluent (100 mM Hepes pH 7.4, 0.2 mM sodium orthovanadate, 0.1% v/v Triton X100, 0.2 mM dithiothreitol). For a typical batch, stock enzyme is diluted 1 in 2000 with enzyme diluent and 50 μL of dilute enzyme is used for each assay well.

A stock of substrate solution was prepared from a random copolymer containing tyrosine, for example Poly (Glu, Ala, Tyr) 6:3:1 (Sigma P3899), stored as 1 mg/ml stock in PBS at −20° C. and diluted 1 in 500 with PBS for plate coating.

On the day before the assay 100 μl of diluted substrate solution was dispensed into all wells of assay plates (Nunc maxisorp 96-well immunoplates) which were sealed and left overnight at 4° C.

On the day of the assay the substrate solution was discarded and the assay plate wells were washed once with PBST (PBS containing 0.05% v/v Tween 20) and once with 50 mM Hepes pH7.4.

Test compounds wete diluted with 10% dimethylsulphoxide (DMSO) and 25 μl of diluted compound was transferred to wells in the washed assay plates. “Total” control wells contained 10% DMSO instead of compound. Twenty five microlittes of 40 mM manganese(II)chloride containing 8 μM adenosine-5′-triphosphate (ATP) was added to all test wells except “blank” control wells which contained manganese(II)chloride without ATP. To start the reactions 50 μl of freshly diluted enzyme was added to each well and the plates were incubated at room temperature for 20 minutes. The liquid was then discarded and the wells were washed twice with PBST. One hundred microlitres of mouse Ig anti-phosphotyrosine antibody (Upstate Biotechnology Inc. product 05-321), diluted 1 in 6000 with PBST containing 0.5% w/v bovine serum albumin (BSA), was added to each well and the plates were incubated for 1 hour at room temperature before discarding the liquid and washing the wells twice with PBST. One hundred microlitres of horse radish peroxidase (HRP)-linked sheep anti-mouse Ig antibody (Amersham product NXA 931), diluted 1 in 500 with PBST containing 0.5% w/v BSA, was added and the plates were incubated for 1 hour at room temperature before discarding the liquid and washing the wells twice with PBST. One hundred microlitres of 2,2′-azino-bis(3-ethylbenzthiazoline-6-sulphonic acid) (ABTS) solution, freshly prepared using one 50 mg ABTS tablet (Boehringer 1204 521) in 50 ml freshly prepared 50 mM phosphate-citrate buffer pH5.0+0.03% sodium perborate (made with 1 phosphate citrate buffer with sodium perborate (PCSB) capsule (Sigma P4922) per 100 ml distilled water), was added to each well. Plates were then incubated for 20-60 minutes at room temperature until the optical density value of the “total” control wells, measuted at 405 nm using a plate reading spectrophotometer, was approximiately 1.0. “Blank” (no ATP) and “total” (no compound) control values were used to determine the dilution range of test compound which gave 50% inhibition of enzyme activity.

(b) In Vitro HUVEC Proliferation Assay

This assay determines the ability of a test compound to inhibit the growth factor-stimulated proliferation of human umbilical vein endothelial cells (HUVEC).

HUVEC cells were isolated in MCDB 131 (Gibco BRL)+7.5% v/v fetal calf serum (PCS) and were plated out (at passage 2 to 8), in MCDB 131+2% v/v FCS+3 μg/ml heparin+1 82 g/ml hydrocortisone, at a concentration of 1000 cells/well in 96 well plates. After a minimum of 4 hours they were dosed with the appropriate growth factor (i.e. VEGF 3 ng/ml, EGF 3 ng/ml or b-FGF 0.3 ng/ml) and compound. The cultures were then incubated for 4 days at 37° C. with 7.5% carbon dioxide. On day 4 the cultures were pulsed with 1 μCi/well of tritiated-thymidine (Amersham product TRA 61) and incubated for 4 hours. The cells were harvested using a 96-well plate harvester (Tomtek) and then assayed for incorporation of tritium with a Beta plate counter. Incorporation of radioactivity into cells, expressed as cpm, was used to measure inhibition of growth factor-stimulated cell proliferation by compounds.

(c) In Vivo Solid Tumour Disease Model

This test measures the capacity of compounds to inhibit solid tumour growth.

CaLu-6 tumour xenografts were established in the flank of female athymic Swiss nu/nu mice, by subcutaneous injection of 1×10⁶ CaLu-6 cells/mouse in 100 μl of a 50% (v/v) solution of Mattigel in serum free culture mediu,. Ten days after cellular implant, mice were allocated to groups of 8-10, so as to achieve comparable group mean volumes. Tumours were measured using vernier calipers and volumes were calculated as: (1×w)×√(1×w)×(π/6), where 1 is the longest diameter and w the diameter perpendicular to the longest diameter. Test compounds were administered orally once daily for a minimum of 21 days, and control animals received compound diluent. Tumours were measured twice weekly. The level of growth inhibition was calculated by comparison of the mean tumour volume of the control group versus the treatment group, and statistical significance determined using a Students' t-test and/or a Mann-Whitney Rank Sum Test. The inhibitory effect of compound treatment was considered significant when p<0.05.

(d) hERG-Encoded Potussium Channle Inhibition Test

This assay determines the ability of a test compound to inhibit the tail current flowing through the human ether-a-go-go-related-gene (hERG)-encoded potassium channel.

Human embryonic kidney (HEK) cells expressing the hERG-encoded channel were grown in Miniium Essential Medium Eagle (EMEM; Sigma-Aldrich catalogue number M2279), supplemented with 10% Foetal Calf Serum (Labtech Internation; product number 4-101-500), 10% M1 serum-free supplement (Egg Technologies; product number 70916) and 0.4 mg/ml Geneticin G418 (Sigma-Aldrich; catalogue number G7034). One ot two days before each experiment, the cells were detached from the tissue culture flasks with Accutase (TCS Biologicals) using standard tissue culture methods. They were then put onto glass coverslips resting in wells of a 12 well plate and covered with 2 ml of the growing media.

For each cell recorded, a glass coverslp containing the cells were placed at the bottom of a Perspex chamber containging bath solution (see below) at ambient temperature (˜20° C.). This chamber was fixed to the stage of an inverted, phase-contrast microscope. Immediately after placing the coverslip in the chamber, bath solution was perfused into the chamber from a gravity-fed reservoir fot 2 minutes at a rate of ˜2 ml/min. After this time, perfusion was stopped.

A patch pipette made from borosilicate glass tubing (GC120F, Harvard Apparatus) using a P-97 micropipette pullet (Sutter Instrument Co.) was filled with pipette solution (see hereinafter). The pipette was connected to the headstage of the patch clamp amplifier (Axopatch 200B, Axon Instruments) via a silver/silver chloride wire. The headstage ground was connected to the earth electrode. This consisted of a silver/silver chloride wire embedded in 3% agar made up with 0.85% sodium chloride.

The cell was recorded in the whole cell configutation of the patch clamp technique. Following “break-in”, wich was done at aholding potention pf−80 mV (set by the amplifier), and appropriate adjustment of series resistance and capacitance controls, electrophysiology software (Clampex, Axon Instruments) was used to set a holding potential (−80 mV) and to deliver a voltage protocol. This prtotocol was applied every 15 seconds and consisted of a 1 s step to +40 mV followed by a 1 s step to −50 mV. The current ressponse to each imposed voltage ptotocol was low pass filtered by the amplifier at 1 kHz. The filtered signal was then acquired, on line, by digitisitg this analogue signal from he amplifier with an analogue to digital converter. The digitised signal was then captured on a computer running Clampex software (Axon Instruments). During the holding potential and the step to +40 mV the current was sampled at 1 kHz. The sampling rate was then set to 5 kHz for the remainder of the voltage protocol.

The compositions, pH and osmolarity of the bath and pipette solution are tabulated below.

| Salt | Pipette (mM) | Bath (mM) |
|---|---|---|
| NaCl | — | 137 |
| KCl | 130 | 4 |
| MgCl₂ | 1 | 1 |
| CaCl₂ | — | 1.8 |
| HEPES | 10 | 10 |
| glucose | — | 10 |
| Na₂ATP | 5 | — |
| EGTA | 5 | — |

| Parameter | Pipette | Bath |
|---|---|---|
| pH | 7.18-7.22 | 7.40 |
| pH adjustment with | 1 M KOH | 1 M NaOH |
| Osmolarity (mOsm) | 275-285 | 285-295 |

The amplitude of the hERG-encoded potassium channel tail current following the step from +40 mV to −50 mV was recorded on-line by Clampex software (Axon Instruments). Following stabilisation of the tail current amplitude, bath solution containing the vehicle for the test substance was applied to the cell. Providing the vehicle application had no significant effect on tail current amplitude, a cumulative concentration effect curve to the compound was then constructed.

The effect of each concentration of test compound was quantified by expressing the tail current amplitude in the presence of a given concentration of test compound as a percentage of that in the presence of vehicle.

Test compound potency (IC₅₀) was determined by fitting the percentage inhibition values making up the concentration-effect to a four parameter Hill equation using a standard data-fitting package. If the level of inhibition seen at the hightest test concentration did not exceed 50%, no potency value was produced and a percentage inhibition value at that concentration was quoted.

Although the pharmacological properties of the compounds of formula I vary with structural change, in general, activity possessed by compounds of the formula I, may be demonstrated at the following concentrations or doses in one or more of the above tests (a), (b) and (c)

Test (a): IC₅₀ in the range, for example, >5 μM;

Test (b): IC₅₀ in the range, for example, 0.001-5μM;

Test (c): activity in the range, for example, 0.1-100 mg/kg;

Example 1 of the present application has IC₅₀ values in the enzyme assay (a) of:
0.029 μM against KDR;
0.49 μM against Flt-1; and
0.072 μM against EGFR.

In the HUVEC assay (b) Example 1 of the present application has IC₅₀ values of: 0.0114 μM with respect to VEGF and 0.1 with respect to EGF.

Example 1 of the present application has an IC₅₀ of 1.5 μM in the hERG assay (d).

According to a further aspect of the invention there is provided a pharmaceutical composition which comprises a compound of the formula I as defined hereinnefore or a pharmaceutically acceptable salt thereof, in association with a pharmaceutically acceptable excipient or carrier.

The composition may be in a form suitable for oral administration, (for example as tablets, lozenges, hard or soft capsules, aqueous or oily suspensions, emulsions, dispersible powders or granules, syrups or elixirs), for adminstration by inhalation (for example as a finely divided powder or a liquid aerosol), for administration by insufflation (for example as a finely divided powder), for parental injection (for example as a sterile solution, suspension or emulsion for intravenous, subcutaneous, intramuscular, intravascular or infusion dosing), for topical administration (for example as creams, ointments, gels, or aqueous or oily solutions or suspensions), or for rectal administration (for example as a suppository). In general the above compositions may be prepared in a conventional manner using conventional excipients.

The compositions of the present invention are advantageously presented in unit dosage form. The compound will normally be administered to a warm-blooded animal at a unit dose within the range 5-5000 mg per square metre body area of the animal, i.e. approximately 0.1-100 mg/kg. A unit dose in the range, for example, 1-100 mg/kg, preferably 1-50 mg/kg is envisaged and this normally provides a therapeutically-effective dose. A unit dose form such as a tablet or capsule will usually contain, for example 1-250 mg of active ingredient.

Accordinig to a further aspect of the present invention there is provided a compound of the formula I or a pharmaceutically acceptable salt thereof as defined hereinbefore for use in a method of treatment of the human or animal od by therapy.

A further feature of the present invention is a compound of formula I, or a pharmaceutically acceptable salt thereof, for use as a medicament, conveniently a compound of formula I, or a pharmaceutically acceptable salt thereof, for use as medicament for producing an antiangiogenic and/or vascular permeability reducing effect in a warm-blooded animal such as a human being.

Thus according to a further aspect of the invention there is provided the use of a compound of the formula I, or a pharmaceutically acceptable salt thereof in the manufacture of a medicament for use in the production of an antiangiogenic and/or vascular permeability reducing effect in a warm-blooded animal such as a human being.

According to a further feature of the invention there is provided a method for producing an antiangiogenic and/or vascular permeability reducing effect in a warm-blooded animal, such as a human being, in need of such treatment which comprises administering to said animal an effective amount of a compound of formula I or a pharmaceuticallu acceptable salt thereof as defined hereinbefore.

As stated above the size of the dose required for the therapeutic or prophylatic treatment of a particular disease state will necessaily be varied depending on the host treated, the route of administation and the severity of the illness being treated. Preferably a daily dose in the range of 0.1-50 mg/kg is employed. However the daily dose will necessarily be varied depending upon the host treated, the particular route of administration, and the severity of the illness being treated. Accordingly the optimum dosage may be determined by the practitioner who is treating any particular patient.

The antiangiogenic and/or vascular permeability reducing treatment defined hereinbeore may be applied as a sole therapy or may involve, in addition to a compound of the invention, one or more other substances and/or treatments. Such conjoint treatment may be achieved by way of the simultaneous, sequential or separate administration of the individual components of the treatment. In the field of medical oncology it is normal practice to use a combination of different forms of treatment to treat each patient with cancer. In medical oncology the other component(s) of such conjoint treatment in addition to the antiangiogenic and/or vascular permeability reducing treatment defined hereinbefote may be: surgery, radiotherapy or chemotherapy. Such chemotherapy may cover three main categories of therapeutic agent:

(i) other antiangiogenic agents such as those which inhibit the effects of vascular endothelial growth factor, (for exanple the anti-vascular endothelial cell growth factor antibody bevacizumab [Avastin™], and those that work by different mechanism from those defined hereinbefore (for example linomide, inhibitors of integrin αvβ3 function, angiostatin, razoxin, thalidomide), and including vascular targeting agents (for example combretastatin phosphate and compounds disclosed in International Patent Applications WO00/40529, WO 00/41669, WO001/92224, WO02/04434 and WO02/08213 and the vascular damaging agents described in International Patent Application Publication No. WO 99/02166 the entire disclosure of which document is incorporated herein by reference, (for example N-acetylcolchinol-O-phosphate));

(ii) cytostatic agents such as antioestrogens (for example tamoxifen, tormifene, raloxifene, droloxifene, iodoxyfene), oesyrogen receptor down regulators (for example fulvestrant), progestogens (for example megetrol acetate), aromatase inhibtors (for example anastrozole, letrazonem, vorazole, exemestane), antiprogestogens, antiandrogens (for example flutamide, nilutamide, bicalutamide, cyproterone acetate), LHRH agonists and antagonists (for example goserelkin acetate, luprolide, buserelin), inhibitrs of 5α-reductase (for example finasteride), anti-invasion agents (for example metalloproteinase inhibitrs like mrimastate and inhibitors of urokinase plasminogen activator receptor function) and inhibitors of growth factor function, (such growth factors include for example platelet derived growth factor and hepatocyte growth factor), such inhibitors include growth factir antibodies growth factor receptor antoibodies, (for example the anti-erbb2 antibody trastuzumab [Herceptin™] and the anti-erbb1 antibody cetuximab [C225]), famesy transferase inhibitors, tyrosine kinase inhibitors for example inhibitors of the epidermal growth factor family (for example EGFR family tyrosine kinase inhibitors such as N-(3-chloro-4-fluorophenyl)-7-methoxy-6(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033)) and serine/threonine kniase inhibitors); and

(iii) antiproliferative/antineoplastic drugs and combinattons theeeof, as used in medical oncology, such as antyimetabolites (for example antifolates like methotrexate, fluropyrinidines like 5-fluoroutacil, tegafur, purine and adenosne analogues, cytosine arabinoside); antotumoir antibiotics (for example anthracyclines like adriamycin, bleomycin, doxorubicin, daunomycine, epirubicin and idarubucin, mitomycin-C, dactinomycin, mithramycin); platinum derivatives (for example cisplatin, carboplatin); alkylating agents (for example nitrogen mustard, melphalan, chlorambucil, busulphan, cyclophosphamide, ifosdamide, nitrosoureas, thiotepa); anrtimitoci agents (for example vinca alkaloids like vincristine, vinblastine, vindesine, vinorelbine, and taxoids like taxol, taxotere); topoisomerase inhibitors (for example epipodophyllotoxins like etoposide and teniposide, amsacrine, topotecan, camptothecin and also irinotecan); also enzymes (for example asparaginase); and thymidylate synthase inhibitors (for example raltitresed); and additional types of chemotherapeutic agent include:

(iv) biological response modifiers (for example interferon);

(v) antiobodies (for example edrecolomab);

(vi) antisense therapies, for example those which are directed to the targets listed above, such as ISIS 2503, an anti-ras antisense;

(vii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and

(viii) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell energy, approaches using transfected immune cells such as cytokine-transfected dendritic cells, approaches using cytokine-transfected tumour cell lines and approaches using anti-idiotypic antibodies.

For example such conjoint treatment may be achieved by way of the simultaneous, sequential or separate administration of a compoumd of formula I as defined hereinbefore, and a vasculur targeting agent described in WO 99/02166 such as N-acetytcolchinol-O-phosphate (Example 1 of WO 99/02166).

It is known from WO 01/74360 that antiangiogenics can be combined with antihypertessives. A compound of the present invenition can also be administered in combination with an antihypertensive. An antihypertensive is an agent which lowers blood pressure, see WO 01/74360 which is incorputated herein by reference.

Thus according to the present invention there is provided a method of treatment of a disease state associated with angiogeneis which comprises the administration of an effective amount of a combination of a compound of the present invention or a pharmaceutically acceptable salt thereof and an anti-hypertensive agent to a warm-blooded animal, such as a human being.

According to a further feature of the present invention there is provided the use of a combination of a compound of the present invention or a pharmaceutically acceptable salt thereof and an anti-hypertensive agent for use in the manufacture of a medicament for the treatment of a disease state associated with angiogenesis in a warm-blooded mammal, such as a human being.

According to a further feature of the present invention there is provided a pharmaceutical composition comprising a compound of the present invention or a pharmaceutilcaly acceptable salt thereof and an anti-hypertensive agent for the treatment of a disease state associated with angiogenesis in a warm-blooded mammal, such as a human being.

According to a further aspect of the present invention there is provided a method for producing an anti-angiogenic and/or vascular penrmability reducing effect in a warm-blooded animal, such as a human being, which comprises administering to said animal an effective amount of a combination of a compound of the present invention or a pharmaceutically acceptable salt thereof and an anti-hypertensive agent.

According to a further aspect of the present invention there is provided the use of a combination of a compound of the present invention or a pharmaceutically acceptable salt thereof and an anti-hypertensive agent for the manufacture of a medicament for produlcing an anti-angiogenic and/or vascular permeability reducing effect in a warm-blooded mammal, such as a hmnan being.

Preferred antihypertensive agents are calcium channel blockers, angiotensin converting enzyme inhibitots (ACE inhibitors), angiotensin II receptor antagonists (A-II antagonists), diuretics, beta-adrenergic recpeotres blockers (β-clockers), vasodilators amd alpha-asdrenergenic receptor blockers (α-blockers). Particluar antihypertensive agents are calcium channel blockers, angiotensin converting enzyme inhibitpors (ACE inhibitors), angiotensin II receptor antagonists (A-II antagonists) and beta-adrengeric receptor blockers (β-blockers), especially calcium channel blockers.

As stated above the compounds defined in the present invention are of interest for their antiangiogenic and/or vascular pereability reducing effects. Such compounds of the invention are expected to be useful in a wide range of disease states including cancer, diabetes, psoriasis, rheumatoid athritis, Kaposi's sarcoma, haemangioma, lymphoedema, acute and chronic nephropathies, atheroma, arterial restenosis, autoimmune diseases, acute inflammation, excessive scar formation and adjesions, endometrosis, dysfunctional uterine bleeding and ocular diseases with retinal vessel proliferation including age-related macular degeneration. Cancer may affect any tissue and includes leukaemia, multiple myeloma and lymphoma. In particular such compounds of the invention are expected to slow advantageously the growth of primary and recurrent solid tumours of, for example, the colon, breast, prostate, lungs and skin. More patticulatly such compounds of the invention are expected to inhibit any form of cancer associated with VEGF inlcuding leukaemia, multiple myeloma and lymphoma and also, for example, the growth of those primary and recurrent solid tumours which are associated with VEGF, especially those tumours which are significantly dependent on VEGF for their growth and spread, including for example, certain tumours of the colon, breast, prostate, lung, vulva and skin.

In another aspect of the present invention compounds of formula I are expected to inhibit the growth of those primary and recurrent solid tumours which are associated with EGF especially those tumours which are significantly dependent on BGF for their growth and spread.

In another aspect of the present invention compoumds of formula are expected to inhibit the growth of those primary and recurrent solid tumours which are associated with both VEGF and EGF especially those tumours which are significantly dependent on VEGF and EGF for their growth and spread, for example non-small cell lung cancer (NSCLC).

In addition to their use in therapeutic medicine, the compounds of formula I and their pharmaceutically acceptable salts are also useful as pharmacological tools in the development and standardisation of in vitro and in vivo test systems for the evaluation of the effects of inhibitors of VEGF receptor tyrosine kinase activity in laboratory animals such as cats, dogs, rabbits, monkeys, rats and mice, as part of the search for new therapeutic agents.

It is to be understood that where the term “ether” is used anywhere in this specification it refers to diethyl ether.

The invention will now be illustrated in the following non-limiting Examples in which, unless otherwise stated:
(i) evaporations were carried out by rotary evaporation in vacuo and work-up procedures were carried out after removal of residual solids such as drying agents by filtration;
(ii) operations were carried out at ambient temperature, that is in the range 18-25° C. and under an atmosphere of an inert gas such as argon;
(iii) column chromatography (by the flash procedure) and medium pressure liquid chromatography (MPLC) were performed on Merck Kieselgel silicon (Art. 9385) or Merck Lichroprep RP-18 (Art. 9303) reversed-phase silica obtained from E. Merck, Darmstadt, Germany;
(iv) yields are given for illustration only and are not necessarily the maximum attainable;
(v) melting points are uncorrected and were determined using a Mettler SP62 automatic melting point apparatus, an oil-bath apparatus or a Koffler hot plate apparatus.

(vi) the structure of the end-products of the formula I were confirmed by nuclear (generally proton) magnetic resonance (NMR) and mass spectral techniques; protn magnetic reasonance chemical shift values were measured on the delta scale and peak multiplicities are shown as follows: s, singlet; d, doublet; t, triplet; m, multiplet; br, broad; q, quartet, quin, quintet;

(vii) intermediates were not generally fully charactetised and purity was assessed by thin layer chromatography (TLC), high-performance liquid chromatography (HPLC), infra-red (IR) or NMR analysis;

(viii) HPLC were run under 2 different conditions:
1) on a TSK Gel super ODS 2 μM 4.6 mm×5 cm column, eluting with a gradient of methanol in water (containing 1% acetic acid) 20 to 100% in 5 minutes. Flow rate 1.4 ml/minute. Detection: U.V. at 254 nm and light scattering detections;
2) on a TSK Gel super ODS 2 μM 4.6 mm×5 cm column, eluting with a gradient of methanol in water (containing 1% acetic acid) 0 to 100% in 7 minutes. Flow rate 1.4 ml/minutes. Detection: U.V. at 254 nm and light scatterig detections.

(ix) petroleum ether refers to that fraction boiling between 40-60° C.

(x) the following abbreviattons have been used:
DMF N,N-dimethylformamide
DMSO dimethylsulphoxide
TFA trifluoracetic acid
THF tetrahydrofuran
LC-MS HPLC coupled to mass spectrametty

### EXAMPLE 1

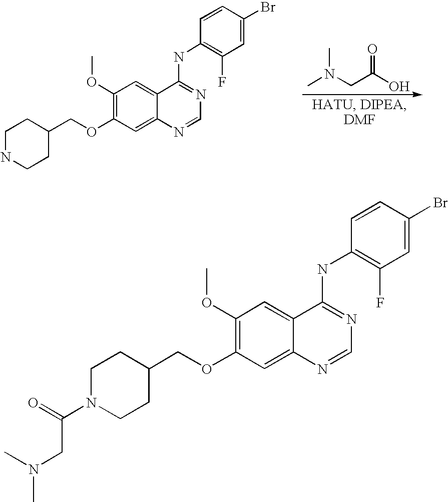

4-(4-Bromo-2-fluoroanilino)-6-methoxy-7-(piperidin-4-ylmethoxy)quinazoline (0.9 g, 1.95 mmol), O-(7-azabenzotriazol-1-yl)-N,N,N′,N′-tetramethyluronium hexafluorophosphate (0.89 g, 2.34 mmol) and N,N-dimethylglycine (241 mg, 2.34 mmol) were dissolved in N,N-dimethylformamide (10 ml) and diisopropylethylamine (0.68 ml, 3.90 mmol) was added. The reaction mixture was stirred at room tempetatute for 3 hours, diluted with ethyl acetate, washed with brine, 2N sodium hydroxide, dried (MgSO₄) and concenttated under reduced pressure. Column chromatography of the residue (2.5% 7N ammonia in methanol/dichloomethane) gave 4-(4-bromo2-fluoroanilino)-7({1-[N,N-dimethylaminp)acetl]piperidin-4-yl}methoxy)-6-methoxyquinaoline (750 mg, 70%) as a white solid.

LC-MS (ESI) 548.0 [M(⁸¹Br)H]⁺

¹H NMR (spectrum): (DMSOd₆) 1.17-1.35 (m, 2H); 1.83 (bt d, 2H); 2.11 (m, 1H); 2.19 (s, 6H); 2.62 (b t, 1H); 3.02 (m, 2H); 3.12 (d, 1H); 3.95 (s, 3H); 4.03 (d, 2H); 4.10 (br d, 1H); 4.40 (br d, 1H); 7.20 (s, 1H); 7.47 (dd, 1H); 7.59 (m, 1H); 7.65 (dd, 1H); 7.80 (s, 1H); 8.36 (s, 1H); 9.51 (s, 1H)

The starting material was prepared as follows:

A mixture of 2-amino-4-benzyloxy-5-methoxybenzamide (10 g, 0.04 mol), (J. Med. Chem. 1977, vol 20 146-149), and Gold's reagent (7.4 g, 0.05 mol) in dioxane (100 ml) was stirred and heated at reflux for 24 hours. Sodium acetate (3.02 g, 0.037 mol) and acetic taid (1.65 ml, 0.029 mol) were added to the reaction mixture and it was heated for a further 3 hours. The mixture was evaporated, water was added to the residue, the solid was filtered off, washed with water and dried (MgSO₄). Recrystallisation from acetic acid gave 7-benzyloxy-6-methoxy-3,4-dihydroquinazolin-4-one (8.7 g, 84%).

10% Palladium on carbon (8.3 g) was added to a suspension of 7-benzyloxy-6-methoxy-3,4-dihydroquinazolin-4-one (50 g, 0.177 mol) in dimethylformamide (800 ml) under nitrogen. Ammonium formate (118.8 g, 1.77 mol) was then added in portions over 5 minutes. The reaction mixture was stirred for one hour at ambient temperature then heated to 80° C. for a further hour. The reaction mixture was filtered hot through diatomaceous earth and the residues washed with dimethylformamide. The filtrate was then concenttated and the residue suspended in water. The pH was adjusted to 7.0 using 2M sodium hydroxide and the resulting mixture was stirred at ambient temperature for one hour. The solid was filtered, washed with water and dried over phosphorus pentoxide yielding 7-hydroxy-6-methoxy-3,4-dihydrquinazolin-4-one as a white solid (20.52 g, 60%).

¹H NMR Spectrum: (DMSOd₆) 3.85 (s, 3H), 6.95 (s, 1H), 7.40 (s, 1H), 1.85 (s, 1H)

MS-ESI: 193 [M+H]+

Pyridine (20 ml) was added to a suspension of 7-hydroxy-6-methoxy-3,4-dihydroquinazolin-4-one (20.5 g, 107 mmol) in acetic anhydride (150 ml, 1.6 mol). The reaction mixture was heated to 120° C. for three hours, during which time the solid dissolved. The reaction mixture was allowed to cool then poured into ice-water (900 ml). The reaction mixture was stirred for one hour then the solid was removed by filtration and dried over phosphorus pentoxide yielding 7-acetoxy-6-methoxy-3,4-dihydroquinazolin-4-one as a white solid (20.98 g, 84%).

¹H NMR Spectrum: (DMSOd₆) 2.25 (s, 3H), 3.85 (s, 3H), 7.40 (s, 1H), 7.60 (s, 1H), 8.00 (s, 1H)

MS-ESI: 235 [M+H]+

7-Acetoxy-6-methoxy-3,4-dihydroquinazolin-4-one (1 g, 4.3 mol) was suspended in thionyl chloride (10.5 ml). One drop of N,N-dimethylformamide was added and the reaction was heated to 80° C. for two hours, during which time the solid dissolved. The reaction mixture was cooled and the thionyl chloride was removed in vacuo. The residue was azeotroped with toluene before being suspended in methylene chloride. A solution of 10% ammonia in methanol (40 ml) was added and the reaction mixture was heated to 80° C. for 15 minutes. After cooling the solvents were removed in vacuo and the residue redissolved in water (10 ml) and the pH adjusted to 7.0 with 2M hydrochloric acid. The resulting solid was filtered, washed with water and dried over phosphorus pentoxide yielding 4-chloro-7-hydroxy-6-methoxyquinazolin as at white solid (680 mg, 75%).

¹H NMR Spectrum: (DMSOd₆) 4.00 (s, 3H), 7.25 (s, 1H), 7.35 (s, 1H), 8.75 (s, 1H)

MS-ESI: 211-213 [M+H]+

While fmaintaining the temperature in the range 0-5° C., a solution of di-tert-butyl dicarbonate (41.7 g, 0.10 mol) in ethyl acetate (75 ml) was added in portions to a solution of ethyl 4-piperidinecarboxylate (30 g, 0.19 mol) in ethyl acetate (150 ml) cooled at 5° C. After stirring for 48 hours at ambient tempetature, the mixture was poured onto water (300 ml). The organic layer was separated, washed successively with water (200 ml), 0.1N aqueous hydrochloric acid (200 ml), saturated sodium hydrogen carbonate (200 ml) and brine (200 ml), dried (MgSO₄) and evaporated to give ethyl 4-(1-(tert-butoxycarbonyl)piperidine)carboxylate (48 g, 98%).

¹H NMR Spectrum: (CDCl₃) 1.25(t, 3H); 1.45(s, 9H), 1.55-170 (m, 2H); 1.8-2.01 (d, 2H); 2.35-2.5 (m, 1H); 2.7-2.95 (t, 2H); 3.9-4.1 (br s, 2H); 4.15 (q, 2H)

A solution of 1M lithium alumiium hydride in THF (13 3ml, 0.133 mol) was added in portions to a solution of ethyl 4-(1-(terf-butoxycarbonyl)piperidine)carboxylate (48 g, 0.19 mol) in dry THF (180 ml) cooled at 0° C. After stirring at 0° C. for 2 hours, water (30 ml) was added followed by 2N sodium hydroxide (10 ml). The precipitate was removed by filtration through diatomaceous earth and washed with ethyl acetate. The filtrate was washed with water, brine, dried (MgSO₄) and evaporated to give 1-(tert-butoxycarbonyl)-4-hydroxymethylpiperidine (36.3 g, 89%).

MS (EI): 215 [M.]+

¹H NMR Spectrum: (CDCl₃) 1.05-1.2 (m, 2H); 1.35-1.55 (m, 10H); 1.6-1.8 (th, 2H); 2.6-2.8 (t, 2H); 3.4-3.6 (t, 2H); 4.0-4.2 (br s, 2H)

4-Chloro-7-hydroxy-6-methoxyquinazoline (1.5 g, 7.12 mmol), tert-butyl 4-(hydroxymethyl)piperidine-1-carboxylate (also known as 1-(tert-butoxycarbonyl)-4-hydroxymethylpiperidine) (1.8 g, 8.55 mmol) and triphenylphosphite (2.2 g, 8.55 mmol) were stirred in dichloromethane (30 ml) and cooled in an ice/water bath.Diisopropyl azodicarboxlyate (1.7 ml, 8.55 mmol) was slowly added and the mixture stirred at room temperature for 3 hours before being concentrated under reduced pressure Column chromatography of the residue (2:1 isohexane/ethyl acetate) gave tert-butyl 4-{[(4-chloro-6-methoxyquinazolin-7-yl)oxy]methyl}piperidihne-1-carboxylate (2.1 g, 72%) as a white solid.

LC-MS (ESI) 408.1 and 410.1 [MH]⁺

¹H NMR (spectrum): (DMSOd₆) 1.33 (m, 2H); 1.52 (s, 9H); 1.90 (d, 2H); 2.16 (m, 1H); 2.89 (m, 2H); 4.11 (m, 5H); 4.22 (d, 2H); 7.5 (s, 1H); 7.55 (s, 1H); 8.98 (s, 1H) tert-Butyl 4-{[4-chloro-6-methoxyquinazolin-7-yl)oxy]methyl}piperidine-1-carboxylate (1.0 g, 2.45 mmol) and 4-bromo-2-fluoroaniline (0.56 g, 2.94 mmol) were stirred in 2-propanol (30 ml) and hydrogen chloride (0.74 ml of a 4M solution in dioxide, 2.94 mmol) was added. The mixture was heated at reflux for 4 hours, cooled and filtered. The solid was dissolved in methanol, placed on an Isolute® SCX column, washed with methanol and then eluted with 7N ammonia in methanol to give 4-(4-bromo-2-fluoroanilino)-6-methoxy-7-(piperidin-4-ylmethoxy)quinazoline (920 mg, 81%) as a pale brown foam.

LC-MS (ESI) 463.0 [M(⁸¹Br)H]⁺

¹H NMR (spectrum): (DMSOd₆) 1.41 (m, 2H); 1.89 (d, 2H); 2.08 (m, 1H); 2.71 (t, 2H); 3.16 (d, 2H); 4.06 (m, 5H); 7.30 (s,1H); 7.62 (m, 2H); 7.17 (d, 1H), 7.93 (s, 1H); 8.46 (s, 1H); 9.68 (br s, 1H)

### EXAMPLE 2

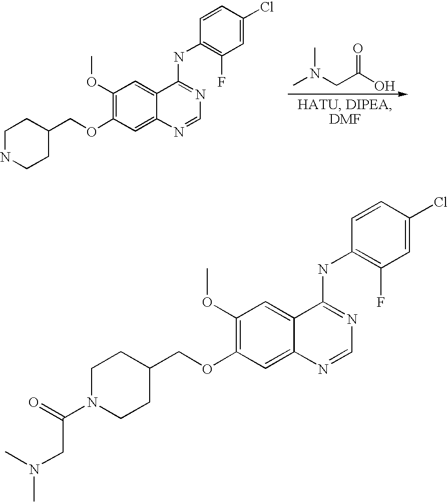

4-(4-Chloro-2-fluoroanilino)-6-methoxy-7-(piperidin-4-ylmethoxy)quinazoline (1.0 g, 2.40 mmol), O-(7-azabenzotriazol-1-yl)-N,N,N′,N′-tetramethyluronium hexafluorophosphate (1.09 g, 2.88 mmol) and N,N-dimethylglycine (297 mg, 2.88 mmol) were dissolved in N,N-dimethylforamide (10 ml ) and diisopropylethylamine (0.84 ml, 4.80 mmol) was added. The reaction mixture was stirred at room temperature for 3 hours, diluted with ethyl acetate, washed with brine, 2N sodium hydroxide, dried (MgSO₄) and concentrated under reduced pressure. Column chromaatography of the residue (2.5% 7N ammonia in methanol/dichloromethane) gave 4-(4-chloro-2-fluoroanilino)-7){1-[N,N-dimethylamino)acetyl]piperidin-4-yl}methoxy)-6-methoxyquinazoline (940 mg, 78%) as a white solid.

LC-MS (ESI) 502.1 and 504.1 [MH]⁺

¹H NMR (spectrum): (DMSOd₆) 1.17-1.35 (m, 2H); 1.83 (br d, 2H); 2.11 (m, 1H); 2.19 (s, 6H); 2.62 (br t, 1H); 3.04 (m, 2H); 3.13 (d, 1H); 3.95 (s, 3H); 4.03 (d, 2H); 4.08 (br d, 1H); 4.40 (br d, 1H); 7.20 (s, 1H); 7.35 (m, 1H); 7.54 (dd, 1H); 7.59 (m, 1H); 7.80 (s, 1H); 8.36 (s, 1H); 9.51 (s, 1H)

The starting material was prepared as follows:

tert-Butyl 4-{[(4-chloro-6-methoxyquinazolin-7-yl)oxy]methyl]piperidine-1-carboxylate (1.0 g, 2.45 mmol), (prepared as described for the starting material in Example 1, and 4-chloro-2-fluoroaniline (0.33 ml, 2.94 mmol) were stirred in 2-propanol (30 ml) and hydrogen chloride (0.74 ml of a 4M solution in dioxane, 2.94 mmol) was added. The mixture was heated at reflux for 4 hours, cooled and filtered. The solid was dissolved in methanol, placed on an Isolute® SCX columm, washed with methanol and then eluted with 7N ammonia in methanol to give 4-(4-chloro-2-fluoroanilino)-6-methoxy-7-(piperidin-4-ylmethoxy)quinazoline (1.0 g, 98%) as a white solid.

LC-MS (ESI) 417.1 and 419.1 [MH]⁺

¹H NMR (spectrum): (DMSOd₆) 1.47 (m, 2H); 1.93 (d, 2H); 2.13 (m, 1H), 2.78 (t, 2H); 3.20 (d, 2H); 4.06 (m, 5H); 7.31 (s, 1H); 7.45 (m, 1H); 7.67 (m, 21); 7.95 (s, 1H); 8.46 (s, 1H); 9.73 (br s, 1H)

### EXAMPLE 3

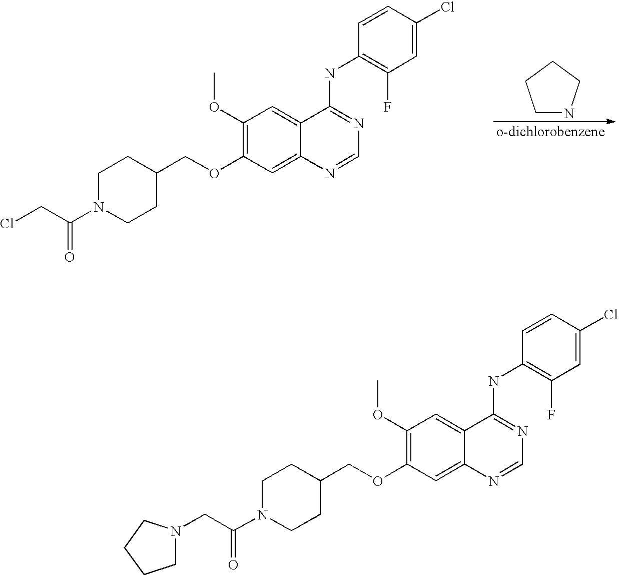 7-{[1-(chloroacetyl)piperidin-4-yl]methoxy}-4-(4-chloro-2-fluoroanilino)-6-methoxyquinazoline (150 mg, 0.30 mmol) was suspended in O-dichlorobenzene (3 ml) and pyrrolidine (63 μl, 0.76 mmol) added. The mixture was heated at 120° C. for 1.5 hours. The reaction mixture was cooled and placed directly onto a silica column, washed with dichloromethane to remove the O-dichlorobenzene and then eluted with 2% 7N ammonia in methanol/dichloromethane to give 4-(4-chloro-2-fluoroanilino)-6-methoxy-7-{[1-(pyrrolidin-1-ylacetyl)piperidin-4-yl]methoxy}quinazoline (115 mg, 72%).
LC-MS (ESI) 528.1 and 530.1 [MH]⁺
¹H NMR (spectrum): (DMSOd₆) 1.25 (m, 2H); 1.69 (m, 4H); 1.92 (br d, 2H); 2.11 (m, 1H); 2.50 (m, 4H); 2.61 (br t, 1H), 3.03 (br t, 1H); 3.17 (d, 1H); 3.34 (d, 1H); 3.95 (s, 3H); 4.06 (m, 3H), 4.39 (br d, 1H): 7.20 (s, 1H), 7.34 (m, 1H); 7,54 (dd, 1H); 1.59 (t, 1H), 7.80 (s, 1H); 8.35 (s, 1H); 9.51 (s, 1H)

The starting material was prepared as follows:

4-(4-Chloro-2-fluoroanilino)-6-methoxy-7-(piperidin-4-ylmethoxy)quinazoline (2.2 g, 4.85 mmol) (prepared as described for the starting material in Example 2) was suspended in methylene chloride (100 ml) and diisopropylethylamine (2.1 ml, 12.1 mmol) was added. Chloroacetyl chloride (0.4 m, 5.34 mmol) was slowly added and the mixture stirred at room temperature for 2 hours. A further 0.5 equivalents of chloroacetyl chloride and diisopropylethyamine were added and the reaction mixture stirred for a further 2 hours. The mixture was washed with 2N hydrochloric acid, dried (MgSO₄) and concentrated under reduced pressure. Column chromatography of the residue (2%-5%-7% methanol/dichloromethane) gave 7-{[1-(chloroacetyl)piperidin-4-yl]methoxy}-4-(4-chloro-2-fluoroanilino)-6-methoxyquinazoline (1.52 g, 62%) as a brown solid.

LC-MS (ESI) 493, 495 and 496.1 [MH]⁺

¹H NMR (spectrum): (DMSO₆) 1.15-1.30 (m, 2H); 1.96 (d, 2H); 2.15 (m, 1H); 2.72 (m, 1H); 3.14 (m, 1H); 3.90 (d, 1H); 3.97 (s, 3H); 4.06 (d, 24); 4.39 (m, 3H); 7.23 (m, 1H); 7.46 (m, 1H); 7.72 (m, 2H); 7.89 (s, 1H); 8.42 (s, 1H); 9.84 (br s, 1H)

### EXAMPLES 4-11

Using an analagous procedure to that described in the preparation of Example 3, 7-{[1-(chloroacetyl)piperidin-4-yl]methoxy}-4(4-chloro-2-fluoroanilino)-6-methoxyquinazoline was reacted with the approrpaite amine to give the compounds described in Table 1.

TABLE 1

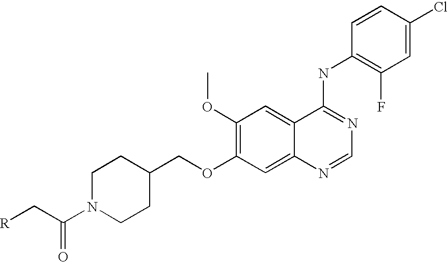

| Example number | R | note |
|---|---|---|
| 4 | 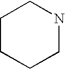 | 1) |
| 5 | 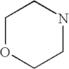 | 2) |
| 6 | 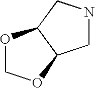 | 3) |
| 7 | 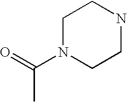 | 4) |
| 8 | 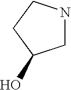 | 5) |
| 9 | 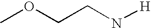 | 6) |
| 10 | Me NH | 7) |
| 11 | 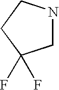 | 8) |

The (3RS,4SR)-3,4-methylenedioxypyrrolidine used as a starting material was prepared as follows:

A solution of di-tert-butyl dicarbonate (Boc₂O, 79.95 g) in ethyl acetate (125 ml) was added dropwise to a stirred mixture of 3-pyrroline (25 g; 65% pure containing pyrrolidine) and ethyl acetate (125 ml) which had been cooled to 0° C. The reaction temperature was maintained at 5-10° C. during the addition. The resultant reaction mixture was allowed to warm to ambient temperature overnight. The reaction mixture was washed successively with water, 0.1N aqueous hydrochloric acid solution, water, a saturated aqueous sodium bicarbonate solution and brine, dried over magnesium sulphate and evaporated. There was thus obtained, as a colorless oil (62 g), a 2:1 mixture of tert-butyl 3-pyrroline-1-carboxylate, ¹H NMR (spectrum): (CDCl₃) 1.45 (s, 9H), 4.1 (d, 4H), 6.75 (m, 2H), and tert-butyl pyrrolidine-1-carboxylate, ¹H NMR (spectrum): (CDCl₃) 1.5 (s, 9H), 1.8 (br s, 4H), 3.3 (br s, 4H).

A solution of the mixture of materials so obtained in acetone (500 ml) was added dropwise to a mixture of N-methylmorpholine-N-oxlde (28.45 g), osmium tetroxide (1 g) and water (500 ml) whilst keeping the reaction temperature below 25° C. The reaction mixture was then stirred at ambient temperature for 5 hours. The solvent was evaporated and the residue was partitioned between ethyl acetate and water. The organic phase was washed with brine, dried over magnesium sulphate and evaporated. The residue was purified by column chromtagraphy on silica using increasingly polar mixtures of petroleum ether (b.p. 40-60° C.) and ethyl acetate as eluent and by further column chromatography on silica using increasingly polar mixtures of methylene chloride and methanol. There was thus obtained tert-butyl (3RS,4SR)-3,4-dihydroxypyrrolidine-1-carboxylate as an oil (34.6 g).

¹H NMR (spectrum): (CDCl₃) 1.45 (s, 9H), 2.65 (m, 2H), 3.35 (m, 2H), 3.6 (m, 2H) 4.25 (m, 2H).

A solution of tert-butyl (3RS,4SR)-3,4-dihydroxypyrrolidine-1-carboxylate (34.6 g) in DMF (400 ml) was cooled to 0-5° C. and sodium hydride (60% dispersion in mineral oil, 0.375 mol) was added portiinwise. The reaction mixture was stirred at 5° C. for 1 hour. Dibromomethane (15.6ml) was added and the reaction mixture was stirred at 5° C. for 30 minutes. The reaction mixture was allowed to warm to ambient temperature and was stirred for 16 hours. The DMF was evaporated and the residue was partitioned between ethyl acetate and water. The organic phase was washed with water and with brine, dried over magnesium sulphate and evaporated. The residue was purified by column chromatography on silica using increasingly polar mixtures of petroleum ether (b.p. 40-60° C.) and ethyl acetate as eluent. There was thus obtained tert-butyl (3RS ,4SR)-3,4-methylenedioxypyrrolidine-1-carboxylate as a colourless oil (19.17 g).

¹H NMR (spectrum): (CDCl₃) 1.45 (s, 9H), 3.35 (m, 2H), 3.75 (br s, 2H) 4.65 (m, 2H), 4.9 (s, 1H), 5.1 (s, 1H).

A cooled 5M solution of hydrogen chloride in isopropanol (150 ml) was added to a solution of tert-butyl (3RS,4SR)-3,4-methytlenedioxypyrrolidine-1-carboxylate (19.7 g) in methylene chloride (500 ml) that was cooled in an ice bath. The reaction mixture was allowed to warm to ambient temperature and was stirred for 4 hours. The solvent was evaporated and the residue was triturated under diethyl ether. The precipitate was collected by filtration, washed with diethyl ether and dried. There was thus obtained (3RS,4SR)-3,4-methylenedioxypyrrolidine hydrochloride as a beige solid (13.18 g).

¹H NMR (spectrum): (DMOSd₆) 3.15 (m, 2H), 3.35 (m, 2H), 4.65 (s, 1H), 4.8 (m, 2H), 5.1 (s, 1H).

The material so obtained was suspended in diethyl ether and a saturated methanolic ammonia solution was added. The resultant mixture was stirred at ambient temperature for 10 minutes. The mixture was filtered and the solvent was evaporated at ambient temperature under vacuum. There was thus obtained (3RS,4SR)-3,4-methylenedioxypyrrolidine which was used without any additional purification.

4) 7-({1-[(4-acetylpiperazin-1-yl)acetyl]piperidin-4-yl}methoxy)-4-(4-chloro-2-fluoroanilino)-6-methoxyquinazoline (70 mg, 39%)

LC-MS (ESI) 585 and 587 [MH]⁺

¹H NMR (spectrum): (DMOSd₆) 1.20 (m, 1H); 1.35 (m, 1H); 1.84 (m,, 2H); 1.98 (s , 3H), 2.12 (m, 1H); 2.37 (m, 2H), 2.43 (m, 2H); 2.63 (m, 1H); 3.08 (m, 2H); 3.30 (d, 1H); 3.42 (m, 4H); 3.95 (s, 3H); 4.05 (m, 3H); 4.39 (d, 1H); 7.20 (s, 1H); 7.35 (d, 1H); 7.54 (dd, 1H); 7.59 (t, 1H); 7.79 (s, 1H); 8.35 (s, 1H); 9.51 (s, 1H)

(5) (3S)-7-({1-[3-hydroxypyrrolidin-1-yl)acetyl]piperidin-4-yl}methoxy)-4-(4-chloro-2-fluoroanilino)-6-methoxyquinazoline (34 mg, 20%)

LC-MS (ESI) 543.9 and 546.0 [MH]⁺

¹H NMR (spectrum): (DMSOd₆) 1.18 (m, 1H); 1.32 (m, 1H); 1.55 (m, 1H); 1.83 (d, 2H); 1.96 (m, 1H); 2.11 (m, 1H); 2.34 (m, 1H); 2.50 (m, 1H); 2.61 (m, 2H); 2.77 (m, 1H); 3.02 (br t, 1H); 3.17 (dd, 1H); 3.30 (dd, 1H); 3.95 (s, 3H); 4.04 (m, 3H); 4.18 (m, 1H); 4.38 (d, 1H); 4.65 (d, 1H); 7.20 (s, 1H); 7.35 (d, 1H); 7.54 (dd, 1H); 7.59 (t, 1H); 7.80 (s, 1H); 8.35 (s, 1H); 9.51 (s, 1H)

6) 4-(4-chloro-2-fluoroanilino)-6-methoxy-7-[(1-([N-(2-methoxyethyl)amino]acetyl}piperidin-4-yl)methoxy]quinazoline (65 mg, 22%)

LC-MS (ESI) 532 and 534 [MH]⁺

¹H NMR (spectrum): (DMSOd₆) 1.74(m, 2H); 1.84 (d, 2H); 2.12 (d, 1H); 2.66 (m, 3H); 3.02 (t, 1H); 3.25 (s, 3H); 3.40 (m, 4H); 3.85 (d, 1H); 3.95 (a, 314); 4.03 (d, 2H); 4.42 (d, 1H); 7.20 (s, 1H); 7.35 (d, 1H); 7.45 (dd, 1H); 7.59 (t, 1H); 7.80 (s, 1H); 8.35 (s, 1H); 9.51 (s, 1H)

7) 4-(4-chloro-2-fluoroanilino)-6-methoxy-7({1-[N-methoylamino)acetyl]pipieridin-4-yl}methoxy)quinazoline (54 mg, 46%)

LC-MS (ESI) 488 and 490 [MH]⁺

¹H NMR (spectrum): (DMSOd₆) 1.12 (m, 2H); 1.83 (d, 2H); 2.12 (m, 1H); 2.29 (s, 3H); 2.65 (m, 1H); 3.02 (t, 1H); 3.30 (dd, 2H); 3.86 (d, 1H), 3.95 (s, 3H); 4.03 (d, 2H); 4.42 (d, 1H); 7.20 (s, 1H); 7.35 (d, 1H); 7.57 (m, 2H); 7.80 (s, 1H); 7.36 (s, 1H); 9.52 (s, 1H)

8) 4-(4-chloro-2-fluoroanilino)-7({1-[3,3-difluoropyrrolidin-1-yl)acetyl]piperidin-4-yl}methoxy)-6-methoxyquinazoline (45 mg, 26%)

LC-MS (ESI) 586.4 and 570.5 [M+Na]⁺

¹H NMR (spectrum): (DMSOd₆) 1.27 (m, 2H); 1.83 (d, 2H); 2.12 (m, 1H); 2.23 (m, 2H); 2.63 (m, 1H); 2.80 (t, 2H); 2.99 (m, 3H); 2.30 (d, 1H); 3.42 (d, 1H); 3.95 (m, 4H); 4.03 (d, 2H); 4.38 (d, 1H); 7.20 (s, 1H); 7.35. (d, 1H); 7.54 (dd, 1H); 7.59 (t, 1H); 7.80 (s, 1H); 8.35 (s, 1H); 9.31 (s, 1H)

### EXAMPLE 12

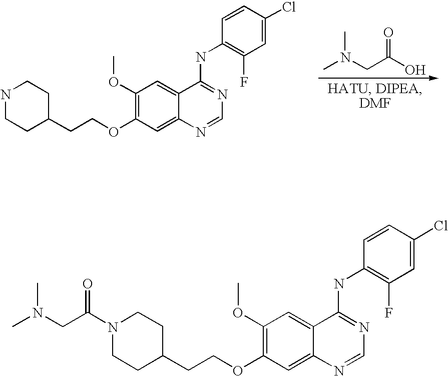

4-(4-Chloro-2-fluoroanilino)-6-methoxy-7-[2-(piperidin-4-yl)ethoxy]quinazoline (310 mg, 0.72 mmol), O-(7-azabenzotriazol-1-yl)-N,N,N′,N′-tetramethyluronium hexafluorophosphate (328 mg, 0.86 mmol) and N,N-dimethylglycine (89 mg, 0.86 mmol) were dissolved in N,N-dimethylformamide (10 ml) and diisopropylethylamine (0.25 ml, 1.44 mmol) was added. The reaction mixture was stirred at room temperature over night, diluted with ethyl acetate, washed with brine (×2), 2N sodium hydroxide, dried (MgSO₄) and concentrated under reduced pressure. Column chromatography of the residue (3% 7N ammonia in methanol/dichloromethane) gave 4-(4-chloro-2-fluoroanilino)-7-(2-{1-[N,N-dimethylamino)acetyl]piperidin-4-yl}ethoxy)-6-methoxyquinazoline (200 mg, 54%) as a white solid.

LC-MS (ESI) 516.1 and 518.1 [MH]⁺

¹H NMR (spectrum) (DMSOd₆) 1.03-1.19 (m, 2H); 1.77 (m, 5H); 2.19 (s, 6H); 2.56 (br t, 1H); 2.99 (m, 2H); 3.14 (br d, 1H); 3.95 (s, 3H); 4.02 (br d, 1H); 4.20 (m, 2H); 4.37 (br d, 1H); 7.22 (s, 1H); 7.35 (d, 1H); 7.54 (dd, 1H); 7.59 (t, 1H); 7.80 (s, 1H); 8.36 (s, 1H); 9.51 (s, 1H)

The starting material was prepared as follows:

4-Chloro-7-hydroxy-6-methoxyquinazoline (1.0 g, 4.75 mmol), (prepared as described for the starting material in Example 1), tert-butyl 4-(2-hydroxyethyl)piperidine-1-carboxylate (1.3 g, 5.70 mmol) and triphenylphosphine (1.5 g, 5.70 mmol) were stirred in dichloromethane (25 ml) and cooled in an ice/water bath. Diisopropyl azodicarboxylate (1.1 ml, 5.70 ml ) was slowly added and the mixture stirred at room temperature over night before being concetrated under reduced pressure. Column chromatography of the residue (2:1 isohexane/ethyl acetate) gave a sticky solid which was suspended in diethyl ether and filtered to give tert-butyl 4-(2-[(4-chloro-6-methoxyquinazolin-7-yl)oxy]ethyl}piperidine-1-carboxylate (1.4 g, 70%) as a white solid.

LC-MS (ESI) 422.0 and 424.0 [MH]⁺

¹H NMR (spectrum): (DMSOd₆) 1.09 (m, 2H); 1.40 (s, 9H); 1.77 (m, 5H); 2.72 (m, 2H); 3.93 (br d, 2H); 4.00 (s, 3H); 4.28 (t, 2H); 7.39 (d, 1H); 7.41 (s, 1H); 9.87 (s, 1H)

tert-Butyl 4-{2-[4-chloro-6-methoxyquinazolin-7-yl)oxy]ethyl}piperidine-1-carboxylate (0.4 g, 0.95 mmol) and 4-chloro-2-fluoroaniline (126 μl, 1.14 mmol) was stirred in 2-propanol (15 ml) and hydrogen chloride (1.2 ml of a 4M solution in dioxane, 4.75 mmol) was added. The mixture was heated at reflux for 1.5 hours, cooled and concetrated under reduced pressure. Column chromatography of the residue (10% 7N ammonia in methanol/dichloromethane) gave 4-(4-chloro-2-fluoroanilino)-6-methoxy-7-[2-(piperidine-4-yl)ethoxy]quinazoline (320 mg, 75%) as a white solid.

LC-MS (ESI) 431.0 and 433.0 [MH]⁺

¹H NMR (spectrum): (DMSOd₆) 1.09 (m, 2H); 1.57 (m, 1H); 1.69 (m, 4H); 2.45 (dt, 2H); 2.92 (br d, 2H); 3.95 (s, 3H); 4.18 (t, 2H); 7.20 (s, 1H); 7.34 (m, 1H); 7.54 (dd, 1H); 7.59 (t, 1H), 7.79 (s, 1H); 8.35 (s, 1H); 9.52 (br s, 1H)

### EXAMPLE 13

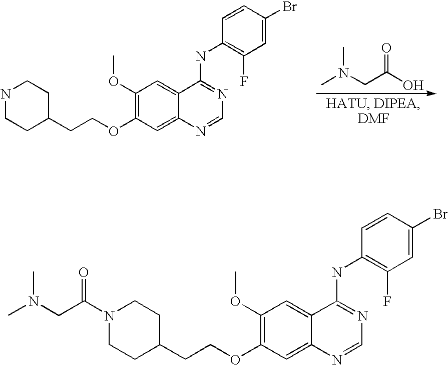

4-(4-Bromo-2-fluoroanilino)-6-methoxy-7-[2-(piperidin-4-yl)ethoxy]quinazoline (330 mg, 6.94 mmol), O-(7-azabenzotriazol-1-yl)-N,N,N,′,N′-tetramethyluronium hexafluorophosphate (317 mg 0.83 mmol) and N,N-dimethylglycine (86 mg, 0.83 mmol) were dissolved in N,N-dimethylforamide (10 ml) and diisopropylethylamine (0.24 ml, 1.39 mmol) was added. The reaction mixture was stirred at room temperature over night, diluted with ethyl acetate, washed with brine (×2), 2N sodium hydroxide, dried (MgSO₄) and concentrated under eeduced pressure. Column chromatography of the residue (3% 7N ammonia in methanol/dichloromethane) gave 4-(4-bromo-2-fluoroanilino)-7-(2-{1-[(N,N-dimethylamino)acetyl]piperidin-4-yl}ethoxy)-6-methoxyquinazoline (330 mg, 85%) as a white solid.

LC-MS (ESI) 562.1 [M(⁸¹Br)H]⁺

¹H NMR (spectrum): (DMSOd₆) 1.03-1.19 (m, 2H), 1.76 (m, 5H); 2.18 (s, 6H); 5.56 (br t, 1H); 2.98 (m, 2H); 3.11 (br d, 1H); 3.95 (s, 3H), 4.03 (br d, 1H); 4.20 (m, 2H); 4.34 (br d, 1H); 7.22 (s, 1H) 7.47 (dd, 1H); 1.54 (t, 1H); 1.65 (dd, 1H); 7.79 (s, 1H); 8.36 (s, 1H); 9.50 (s, 1H)

The starting material was prepared as follows:

tert-Butyl 4-{2-[(4-chloro-6-methoxyquinazolin-7-yl)oxy]ethyl}piperidine-1-carboxylate (0.4 g, 0.95 mmol), (prepared as described for the starting material in Example 12), and 4-bromo-2-fluoroaniline (216 mg, 1.14 mmol) were stirred in 2-propanol (15 ml ) and hydrogen chloride (1.2 ml of a 4M solution in dioxide, 4.75 mmol) was added. The mixture was heated at reflux for 1.5 hours, cooled and concentrated under reduced pressure. Column chromatography of the residue (10% 7N ammonia in methanol/cichloromethane) gave 4-(4-bromo-2-fluroanilino)-6-methoxy-7-[2-piperidin-4-yl)ethoxy]quinazoline (339 mg, 75%) as a white solid.

LC-MS (ESI) 472.9 and 474.9 [M−H]⁺

¹H NMR (spectrum): (DMSOd₆) 1.10 (m, 2H): 1.58 (m, 1H); 1.69 (m, 4H); 2.46 (dt, 2H); 2.92 (br d, 2H); 3.94 (s, 3); 4.18 (t, 2H); 7.20 (s, 1H); 7.46 (m, 1H); 7.53 (t, 1H); 7.59 (dd, 1H); 7.79 (s,1H); 8.35 (s, 1H); 9.51 (br s, 1H)

### EXAMPLE 14

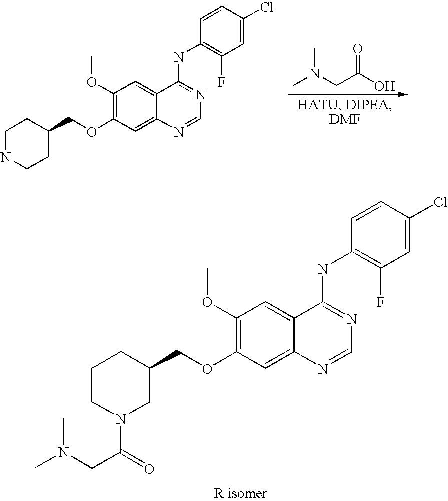

4-(4-Chloro-2-fluoroanilino)-6-methoxy-7-[(3R)-piperidin-3-ylmethoxy]quinazoline (150 mg, 0.36 mmol), O-(7-azabenzotriazol-1-yl)-N,N,N′,N′-tetramethyluronium hexafluorophosphate (164 mg, 0.43 mmol) and N,N-dimethylglycine (45 mg, 0.43 mmol) were dissolved in N,N-dimethylformamide (4 ml) an diisopropylethylamine (0.125 μl, 0.72 mmol) was added. The reaction mixture was stirred at room temperature for 2 hours, diluted with ethyl acetate, washed with brine (×2), 2N sodium hydroxide, dried (MgSO₄) and concetrated under reduced pressure. Colum chromatography of the residue (2.5% 7N ammonia in methanol/dichloromethane) gave 4-(4-chloro-2-fluoroanilino)-7({(3R)-1-[(N,N-dimethylamino)acetyl]piperidin-3-yl}methoxy)-6-methoxyquinazoline (138 mg, 76%) as a white solid.

LC-MS (ESI) 502 and 504 [MH]⁺

¹H NMR (spectrum): (DMSOd₆ at 373° K.) 1.45 (m, 2H); 1.71 (m, 1H); 1.91 (m, 1H); 2.08 (m, 1H); 2.21 (s, 6H); 3.05 (m, 4H), 3.95 (m, 4H); 4.10 (m, 2H); 4.20 (m, 1H); 7.21 (s, 1H); 7.30 (d, 1H); 7.40 (d, 1H); 7.65 (t, 1H); 7.80 (s, 1H); 8.37 (s, 1H); 9.15 (s, 1H)

The starting material wa sprepared as follows:

4-Chloro-7-hydroxy-6-methoxyquinazoline (250 mg, 1.19 mmol), (prepared as described for the starting material in Example 1), tert-buty (3R)-3-(hydroxymnethyl)pperidine-1-carboxylate (307 mg, 1.42 mmol) and triphenylphosphine (374 mg, 1.42 mmol) were stirred in dichloromethane (12ml) and cooled in an ice/water bath. Diisopropyl azodicarboxylate (280 μl, 1.42 mmol) in dichloromethane (2 ml) was slowly added and the mixture was stirred at room temperature for 2.5 hours before being concetrated under reduced pressure. Column chromatography of the residue (2:1 isohexane/ethyl acetate) gave tert-butyl (3R)-3-{[(4-chloro-6-methoxyquinazolin-7-yl)oxy]methyl}piperidine-1-carboxylate (400 mg, 82%) as a viscous oil.

LC-MS (ESI) 408 and 410 [MH]⁺

¹H NMR (spectrum): (DMSOd₆) 1.36 (m, 11H); 1.60 (m, 1H); 1.87 (m, 1H); 1.99 (m, 1H); 2.90 (m, 1H); 3.72 (m, 1H); 4.01 (m, 7H); 7.40 (s, 1H); 7.46 (s, 1H); 8.87 (s, 1H) tert-Butyl (3R)-3-{[(4-chloro-6-methoxyquinazolin-7-yl)oxy]methyl}piperidine-1-carboxylate (400 mg, 0.98 mmol) and 4-chloro-2-fluoroaniline (130 μl, 1.18 mmol) were stirred in 2-propanol (12 ml) and hydrogen chloride (294 μl of a 4M solution in dioxane, 1.18 mmol) was added. The mixture was heated at reflux for 4 hours, cooled and filtered. The solid was dissolved in methanol, absorbed onto an Isolute® column, washed with methanol and eluted with 7N ammonia in methanol to give 164 mg of first batch of product as a white solid. Column chromatography of the concentrated filtrate (10% 7N ammonia in methanol/dichloromethane) gave a further 41 mg of 4-(4-chloro-2-fluoroanilino)-6-methoxy-7-[(3R)-piperidin-3-ylmethoxy]quinazoline which was combined with the first batch (205 mg in total, 50%).

LC-MS (ESI) 417 and 419 [MH]⁺

¹H NMR (spectrum): (DMSOd₆) 1.25 (m, 1H); 1.41 (m, 1H); 1.59 (m, 1H); 1.84 (m, 1H); 1.95 (m, 1H); 2.38 (t, 1H); 2.50 (m, 1H); 2.86 (d, 1H); 3.07 (d, 1H); 3.95 (s, 3H); 4.00 (d, 2H); 7.18 (s, 1H); 7.34 (d, 1H); 7.54 (dd, 1H); 7.59 (t, 1H); 7.79 (s, 1H); 8.35 (s, 1H); 9.51 (s, 1H)

### EXAMPLE 15

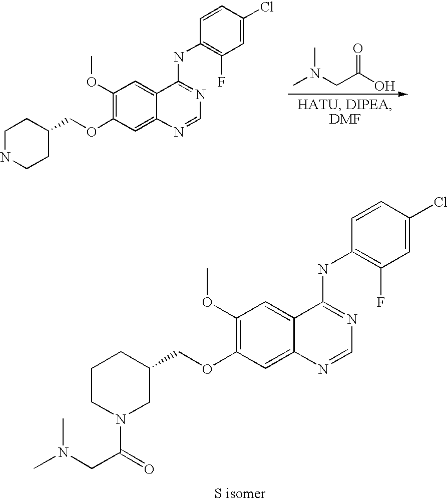

4-(4-Chloro-2-fluoroanilino)-7-({(3S)-1-[(N,N-dimethylamino)acetyl]piperidin-3-yl}methoxy)-6-methoxyquinazoline was prepared using an analogous procedure to that described in Example 14.

LC-MS (ESI) 502 and 504 [MH]⁺

¹HMR (spectrum): (DMSOd₆ at 373° K.) 1.45 (m, 2H); 1.71 (m, 1H); 1.91 (m, 1H); 2.08 (m, 1H); 2.21 (s, 6H); 3.05 (m, 4H); 3.95 (m, 4H); 4.10 (m, 2H); 4.20 (m, 1H); 7.21 (s, 1H); 7.30 (d, 1H); 7.40 (d, 1H); 7.65 (t, 1H); 7.80 (s, IH); 8.37 (s, 1H); 9.15 (s, 1H)

The starting material was prepared as follows:

4-Chloro-7-hydroxy-6-methoxyquinazoline was reacted with (3S)-3-(hydroxymethyl)piperidine-1-carboxylate using an analogous procedure to that described for the starting material in Example 14 to give tert-butyl (3S)-3-{[(4-chloro-6-methoxyquinazolin-7-yl)oxy]methyl}piperidine-1-carboxylate

LC-MS (ESI) 408 and 410 [MH]⁺

¹H NMR (spectrum): (DMSOd₆) 1.36 (m, 11H); 1.60 (m, 1H); 1.87 (m, 1H); 1.99 (m, 1H); 2.90 (m, 1H); 3.72 (m, 1H); 4.01 (m, 7H); 7.40 (s, 1H); 7.46 (s, 1H); 8.87 (s, 1H) 4-(4-Chloro-2-fluoroanlino)methoxy-7-[(3S)-piperidin-3-ylmethoxy]quinazoline was prepared using an analogous procedure to that described for the starting material in Example 14.

LC-MS (ESI) 417 and 419 [MH]⁺

¹H NMR (spectrum): (D)MSOd₆) 1.25 (m, 1H); 1.41 (m, 1H); 1.59 (m, 1H); 1.84 (m, 1H); 1.95 (m, 1H); 2.38 (t, 1H); 2.50 (m, 1H); 2.86 (d, 1H); 3.07 (d, 1H); 3.95 (s, 3H); 4.00 (d, 2H); 7.18 (s, 1H); 7.34 (d, 1H); 7.54 (dd, I1); 7.59 (t, 1H); 7.79 (s, 1H); 8.35 (s, 1H); 9.51 (s, 1H)

### EXAMPLE 16

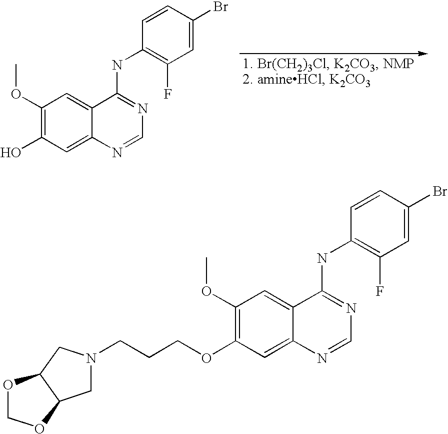

4-(4-Bromo-2-fluoroanilino)-7-hydroxy-6-methoxyquinazoline (986 mg, 2.71 mmol) and potassium carbonate (412 mg, 2.98 mmol) were stirred in 1-methylpyrrolidinone (10 ml) and 1-bromo-3-chloropropane (295 μl, 2.98 mmol) added. The mixture was stirred at 90° C. for 2 hours. (3aR,6aS)-Tetrahydro-3aH-[1,3]dioxolo[4,5-c]pyrrole hydrochloride (452 mg, 2.98 mmol), (prepared as described for the starting material in Example 6), potassium carbonate (412 mg, 2.98 mmol) and a catalytic amount of potassium iodide were added and the mixture heated at 90° C. for a further 3 hours. The mixture was cooled and partitioned between water and dichloromethane. The organic layer was dried (MgSO₄) and concentrated and the residue purified by a combination of column chromatography (1% 1N ammonia in methanol/dichloromethane) and preparative BPLC to give 4-(4-bromo-2-fluoroanilino-6-methoxy-7-{3-[(3aR,6aS)-tetrahydro5H-[1,3]dioxolo[4,5-c]pyrrol-5-yl]propoxy}quinazoline (276 mg, 23%) as a white solid.

LC-MS (ESI) 520.9 [M(⁸¹Br)H]⁺

¹H NMR (spectrum): (DMSOd₆) 1.95 (m, 2H); 2.15 (brd, 2H); 2.42 (m, 2H); 3.01 (d, 2H); 3.95 (s, 3H); 4.17 (t, 2H); 4.57 (m, 2H); 4.86 (s, 1H); 4.95 (s, 1H); 7.17 (s, 1H); 7.47 (m, 1H); 7.54 (t, 1H); 7.65 (dd, 1H); 7.80 (s, 1H); 8.36 (s, 1H); 9.51 (s, 1H).

The starting material was prepared as follows:

A mixture of 2-amino-4-benzyloxy-5-methoxybenzamide (J. Med. Chem. 1977, vol 20, 146-149, 10 g, 0.04 mol) and Gold's reagent (7.4 g, 0.05 mol) in dioxane (100 ml) was stirred and heated at reflux for 24 hours. Sodium acetate (3.02 g, 0.037mol) and acetic acid (1.65 ml, 0.029 mol) were added to the reaction mixture and it was heated for a further 3 hours. The mixture was evaporated, water was added to the residue, the solid was filtered off, washed with water and dried (MgSO₄). Recrystailisation from acetic acid gave 7-benzyloxy-6-methoxy-3,4-dihydroquinazolin-4-one (8.7 g, 84%).

A mixture of 7-benzyloxy-6-methoxy-3,4-dihydroquinazolin-4-one (2.82 g, 0.01 mol), thionyl chloride (40 ml) and DMF (0.28 ml) was stirred and heated to reflux for 1 hour. The mixture was evaporated, the residue was taken up in toluene and evaporated to dryness to give 7-benzyloxy-4-chloro-6-methoxyquinazoline (3.45 g).

A solution of 7-benzyloxy-4-chloro-6-methoxyquinazoline (8.35 g, 27.8 mmol) and 4 bromo-2-fluoroaniline (5.65 g, 29.7 mmol) in 2-propanol (200 ml) was heated at reflux for 4 hours. The resulting precipitate was collected by filtration, washed with 2-propanol and then ether and dried under vacuum to give 7-benzyloxy-4-(4-bromo-2-fluoroanilino)-6-methoxyquinazoline hydrochloride (9.46 g, 78%).

¹H NMR Spectrum: (DMSOd₆; CD₃COOD) 4.0 (s, 3H); 5.37(s, 2H); 7.35-7.5 (m, 4H); 7.52-7.62 (m, 4H); 7.8 (d, 1H); 8.14 (9s, 1H); 8.79 (s, 1H)

MS−ESI: 456 [MH]⁺

| | | | | |
|---|---|---|---|---|
| Elemental analysis: | Found | C 54.0 | H 3.7 | N 8.7 |
| C₂₂H₁₇N₃O₂BrF0.9HCl | Requires | C 54.2 | H 3.7 | N 8.6% |

A solution of 7-benzyloxy-4-(4-bromo-2-fluoroanilino)-6-methoxyquinazoline hydrochloride (9.4 g, 19.1 mmol) in ETA (90 ml) was heated at reflux for 50 minutes. The mixture was allowed to cool and was poured on to ice. The resulting precipitate was collected by filtration and dissolved in methanol (70 ml). The solution was adjusted to pH9-10 with concentrated aqueous ammonia solution. The mixture was concentrated to half initial volume by evaporation. The resulting precipitate was collected by filtration, washed with water and then ether, and dried under vacuum to give 4-(4-bromo-2-fluoroanilino)7-hydroxy-6-methoxyquinazoline (5.66 g, 82%).

¹H NMR Spectrum: (DMSOd₆; CD₃COOD) 3.95(s, 3H); 7.09(s, 1H); 7.48(s, 1H); 7.54(t, 1H); 7.64(d, 1H); 7.79(s, 1H); 8.31(s, 1H)

MS−ESI: 366 [MH]⁺

| | | | | |
|---|---|---|---|---|
| Elemental analysis: | Found | C 49.5 | H 3.1 | N 11.3 |
| C₁₅H₁₁N₃O₂BrF | Requires | C 49.5 | H 3.0 | N 11.5% |

### EXAMPLE 17

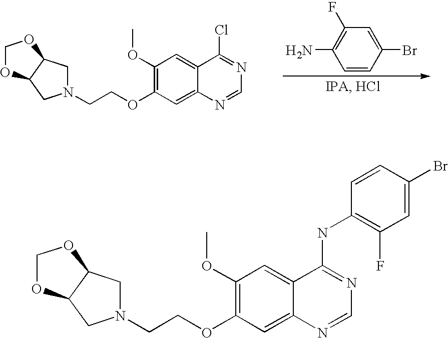

4-Chloro-6-methoxy-7- {2-[(3aR,6aS)-tetrahydro5H-[1,3]dioxolo[4,5-c]pyrrol-5-yl]ethoxy)quinazoline (270 mg, 0.77 mmol) was suspended in 2-propanol-(10 ml) and 4 bromo-2-fluoroaniline (175 mg, 0.92 mmol) added. Hydrogen chloride (230 μl of a 4M solution in dioxane, 0.92 mmol) was added and the mixture heated at refiux for 1.5 hours, cooled and the solid filtered off. The solid was dissolved in 7M ammonia in methanol, concentrated under reduced pressure, water added and the solid filtered off and dried to give 4(4bromo-2-fluoroanilino)-6-methoyx-7-12-[(3aR,6aS)-tetrabydro-5H-[1,3]dioxolo[4,5-c]pyrrol-5-yl]ethoxy}quinazoline (295 mg, 76%) as a white solid.

LC-MS (ESI) 506.9 [M(⁸¹Br)H]⁺

¹H NMR (spectrum): (DMSOd₆) 2.28 (br d, 2H); 2.80 (t 2H); 3.12 (d, 2H); 3.95 (s, 3H); 4.24 (t, 2H); 4.56 (m, 2H); 4.82 (s, 1H); 4.97 (s, 1H); 7.23 (s,-lH); 7.47 (m, 1H); 7.54 (t, 1H); 7.65 (dd, 1H); 7.80 (s, 1H); 8.36 (s, 1H); 9.51 (s, 1H)

The starting material was prepared as follows:

(3aR,6aS)-Tetrahydro-3aH-[1,3]dioxolo[4,5-c]pyrrole hydrochloride (0.7 g, 4.62 mmol), (prepared as described for the starting material in Example 6), potassium carbonate (1.6 g, 11.5 mmol) and 2-bromoethanol (0.33 ml, 4.62 mmol) were heated in acetonitrile (30 ml) at reflux for 2 hours. The mixture was cooled, filtered and concentrated under reduced pressure. Column chromatography of the residue (5% methanol/dichloromethane) gave a pale orange oil which was dissolved in methanol, absorbed onto an Isolute® SCX column, washed with methanol and eluted with 7N ammonia in methanol to give 2-[(3aR,6aS)-tetrahydro-5H-[ 1,3]dioxolo[4,5-c]pyrrol-5-yl]ethanol (313 mg, 43%) as a pale yellow oil.

¹H NMR (spectrum): (CDCl₃) 2.29 (m, 3H); 2.59 (t, 2H); 3.17 (d, 2H); 3.63 (t, 2H); 4.60 (m, 2H); 4.92 (s, 1H); 5.09 (s, 1H)

4-Chloro-7-hydroxy-6-methoxyquinazoline (330 mg, 1.57 mmol), (prepared as described for the starting material in Example 1), 2-[(3aR,6aS)-tetrahydro-5H-[1,3]dioxolo[4,5-c]pyrrol-5-yl]ethanol (300 mg, 1.88 mmol) and triphenylphosphine (494 mg, 1.88 mmol) were stirred in dichloromethane (10 ml) and cooled in an ice/water bath. Diisopropyl azodicarboxylate (371 μl, 1.88 mmol) in dichloromethane (2 ml) was slowly added and the mixture stirred at room temperature for 3 hours before being concentrated under reduced pressure. Column chromatography of the residue (1%-2% methanol/dichloromethane) gave 4-chloro-6-methoxy-7-{2-[(3aR,6aS)-tetrahydro-5H-[1,3]dioxolo[4,5-c]pyrrol-5-yl]ethoxy}quinazoline (280 mg, 51%) as a white solid.

LC-MS (ESI) 352 and 354 [MH]⁺

¹H NMR (spectrum): (DMSOd₆) 2.28 (d, 2H); 2.82 (t, 21H); 3.12 (d, 2H); 4.01 (s, 3H); 4.33 (t, 2H); 4.56 (m, 2H); 4.81 (s, 1H); 4.96 (s, 1H); 7.41 (s, 1H); 7.50 (s, 1H); 8.88 (s, 1H)

### EXAMPLE 18

The following illustrate representative pharmaceutical dosage forms containing the compound of formula I, or a pharmaceutically acceptable salt thereof (hereafter compound X), for therapeutic or prophylactic use in humans:

| (a) | Tablet I | mg/tablet |
|---|---|---|
| | Compound X | 100 |
| | Lactose Ph.Eur | 182.75 |
| | Croscarmellose sodium | 12.0 |
| | Maize starch paste (5% w/v paste) | 2.25 |
| | Magnesium stearate | 3.0 |
| (b) | Tablet II | mg/tablet |
| | Compound X | 50 |
| | Lactose Ph.Eur | 223.75 |
| | Croscarmellose sodium | 6.0 |
| | Maize starch | 15.0 |
| | Polyvinylpyrrolidone (5% w/v paste) | 2.25 |
| | Magnesium stearate | 3.0 |
| (c) | Tablet III | mg/tablet |
| | Compound X | 1.0 |
| | Lactose Ph.Eur | 93.25 |
| | Croscarmellose sodium | 4.0 |
| | Maize starch paste (5% w/v paste) | 0.75 |
| | Magnesium stearate | 1.0 |
| (d) | Capsule | mg/capsule |
| | Compound X | 10 |
| | Lactose Ph.Eur | 488.5 |
| | Magnesium stearate | 1.5 |
| (e) | Injection I | (50 mg/ml) |
| | Compound X | 5.0% w/v |
| | 1M Sodium hydroxide solution | 15.0% v/v |
| | 0.1M Hydrochloric acid | |
| | (to adjust pH to 7.6) | |
| | Polyethylene glycol 400 | 4.5% w/v |
| | Water for injection to 100% | |
| (f) | Injection II | 10 mg/ml) |
| | Compound X | 1.0% w/v |
| | Sodium phosphate BP | 3.6% w/v |
| | 0.1M Sodium hydroxide solution | 15.0% v/v |
| | Water for injection to 100% | |
| (g) | Injection III | (1 mg/ml, buffered to pH6) |
| | Compound X | 0.1% w/v |
| | Sodium phosphate BP | 2.26% w/v |
| | Citric acid | 0.38% w/v |
| | Polyethylene glycol 400 | 3.5% w/v |
| | Water for injection to 100% | |

Note
The above formulations may be obtained by conventional procedures well known in the pharmaceutical art. The tablets (a)-(c) may be enteric coated by conventional means, for example to provide a coating of cellulose acetate phthalate.

1. A compound of the formula I: 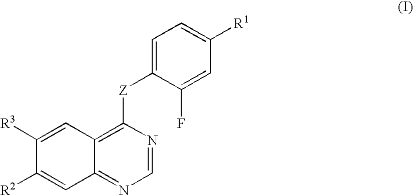
wherein:
Z is —NH—, —O— or —S—;
R¹ represents bromo or chloro;
R³ represents C₁₋₃alkoxy or hydrogen,
R² is selected from one of the following three groups:
(i) Q¹X¹-
wherein X¹ represents —O—, —S— or —NR⁴— wherein R⁴ is hydrogen, C₁₋₃alkyl or C₁₋₃alkoxyC₂₋₃alkyl and Q¹ is selected from one of the following ten groups:
1) Q² (wherein Q² is a 5-6-membered saturated or partially unsaturated heterocyclic group with 1-2 heteroatoms, selected independently from O, S and N, which heterocyclic group bears at least one substituent selected from C₂₋₅alkenyl, C₂₋₅alkynyl, C₁₋₆fluoroalkyl, aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, C₁₋₆fluoroalkanoyl, carbaminoylC₁₋₆alkyl, C₁₋₄alkylcarbamoylC₁₋₆alkyly, di(C₁₋₄alkyl)carbamoylC₁₋₆alkyl, C₁₋₆alkylsulphonyl and C₁₋₆fluoroalksulphonyl and which heterocyclic group may optionally bear a fuirther 1 or 2 substituents selected from C₂₋₅alkenyl, C₂₋₅alkynyl, C₁₋₆fluoroalkyl, C₁₋₆alkanoyl, aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, C₁₋₆fluoroalkanoyl, carbamoyl, C₁₋₄alkylcarbamoyl, di(C₁₋₄alkyl)carbamoyl, carbamoylC₁₋₆alkyl, C₁₋₄alkylcarbamoylC₁₋₆alkyl, di(C₁₋₄alkyl)carbamoylC₁₋₆alkyl, C₁₋₆alkylsulphonyl, C₁₋₆fluoroalkylsulphonyl, oxo, hydroxy, halogeno, cyano, C₁₋₄cyanoalkyl, C_{1-alkyl, C}₁₋₄hydroxyalkyl, C₁₋₄alkoxy, C₁₋₄alkoxyC₁₋₄alkyl, C₁₋₄alkylsulphonylC₁₋₄alkyl, C₁₋₄alkoxycarbonyl, C₁₋₄aminoalkyl, C₁₋₄akylamino, di(C₁₋₄alkyl)amino, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g} ring D (wherein f is 0 or 1, g is 0 or 1 and ring D is a 5-6-membered saturated or partially unsaturated heterocyclic group with 1-2 heteroatoms, selected independently from O, S and N, which cyclic group may bear one or more substituents selected from C₁₋₄alkyl),
or Q² bears a single substituent selected from methylenedioxy and ethylenedioxy); with the proviso that if Q¹ is Q² and X¹ is —O— then Q² must bear at least one substituent selected from C₂₋₅alkenyl, C₂₋₅alkynyl, C₁₋₄alkoxyC₁₋₄alkylaminoC₂₋₆alkanoyl, carbamoylC₁₋₆alkyl, C₁₋₄alkylcarbarnoylC₁₋₆alkyl, and di(C₁₋₄alkyl)carbamoylC₁₋₆alkyl and optionally may bear a further 1 or 2 substituents as defined herein;
2) C₁₋₅alkylW¹Q² (wherein W¹ represents —O—, —S—, —SO—, —SO₂—, —C(O)—, —OC(O)—, —NQ³C(O)—, —C(O)NQ⁴—, —SO₂NQ⁵—, —NQ⁶SO₂— or —NQ⁷- (wherein Q³, Q⁴, Q⁵, Q⁶ and Q⁷ each independently represents hydrogen, C₁₋₃alkyl, C₁₋₃alkoxyC₂₋₃alkyl, C₂₋₅alkenyl, C₂₋₅alkynyl or C₁₋₄haloalkyl) and Q² is as defined herein;
3) C₁₋₅alkylQ² (wherein Q² is as defined herein);
4) C₂₋₅alkenylQ² (wherein Q² is as defined herein);
5) C₂₋₅alkynylQ² (wherein Q² is as defined herein);
6) C₁₋₄allkylW²C₁₋₄alkylQ² (wherein W² represents —O—, —S—, —SO—, —SO₂—, —C(O)—, —OC(O)—NQ⁸C(O)—, —C(O)NQ⁹—, —SO₂NQ¹⁰—, —NQ¹¹SO₂— or —NQ¹²— (wherein Q⁸, Q⁹, Q¹⁰, Q¹¹ and Q¹² each independently represents hydrogen, C₁₋₃alkyl, C₁₋₃alkoxlC₂₋₃alkyl, C₂₋₅alkenyl, C₂₋₅alkynyl or C₁₋₄haloalkyl) and Q² is as defined herein);
7) C₂₋₅alkenylW²C₁₋₄alkylQ² (wherein W² and Q² are as defined herein);
8) C₂₋₅alkynylW²C₁₋₄alkylQ² (wherein w² and Q² are as defined herein);
9) C₁₋₄alkylQ¹³(C₁₋₄alkyl)ⱼ(W²)ₖQ¹⁴ (wherein W² is as defined herein, j is 0 or 1, k is 0 or 1, and Q¹³ and Q¹⁴ are each independently selected from hydrogen, C₁₋₃alkyl, cyclopentyl, cyclohexyl and a 5-6-membered saturated or partially unsaturated heterocyclic group with 1-2 heteroatoms, selected independently from O, S and N, which C₁₋₃alkyl group may bear 1 or 2 substituents selected from oxo, hydroxy, halogeno and C₁₋₄alkoxy and which cyclic group may bear 1, 2 or 3 substituents selected from C₂₋₅alkenyl, C₂₋₅alkynyl C₁₋₆fluoroalkyl, C₁₋₆alkanoyl, aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, C₁₋₆fluoroallanoyl, carbamoyl, C₁₋₄alkylcarbamoyl, di(C₁₋₄alkyl)carbamoyl, carbamoylC₁₋₆alkyl, C₁₋₄alkylcarbamoylC₁₋₆alkyl, di(C₁₋₄alkyl)carbamoylC₁₋₆alkyl, C₁₋₆akylsulphonyl, C₁₋₆fluoroalqylsulphonyl, oxo, hydroxy, halogeno, cyano, C₁₋₄cyanoalkyl, C₁₋₄alkyl, C₁₋₄hydroxyallyl, C₁₋₄alkoxy, C₁₋₄alkoxyC₁₋₄alkyl, C₁₋₄alkylsulphonylC₁₋₄akyl, C₁₋₄alkoxycarbonyl, C₁₋₄aminoalkyl, C₁₋₄alkylamio, di(C₁₋₄alkyl)amino, C₁₋₄alkylaminoC₁₋₄alklyl, di(C₁₋₄alkyl)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g} ring D (wherein f is 0 or 1, g is 0 or 1 and ring D is a 5-6-membered saturated or partially unsaturated heterocyclic group with 1-2 heteroatoms, selected independently from O, S and N, which heterocyclic group may bear one or more substituents selected from C₁₋₄alkyl), with the provisos that Q¹³ cannot be hydrogen and one or both of Q¹³ and Q¹⁴ must be a 5-6-membered saturated or partially unsaturated heterocyclic group as defined herein which heterocyclic group bears at least one substituent selected from C₂₋₅alkenyl, C₂₋₅alkynyl, C₁₋₆-fluoroalkyl, C₁₋₆alkanoyl, aminoC₂₋₅alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, C₁₋₆fluoroalkanoyl, carbamoyl, C₁₋₄alkylcarbamoyl, di(C₁₋₄alkyl)carbamoyl, carbamoylC₁₋₆alkyl, C₁₋₄alkylcarbamoylC₁₋₆alkyl, di(C₁₋₄alkyl)carbamoylC₁₋₆alkyl, C₁₋₆alkylsulphonyl and C₁₋₆fluoroalkylsulphonyl and which heterocyclic group optionally bears 1 or 2 further substituents selected from those defined herein); and
10) C₁₋₄alkyl wherein Q¹³ is as defined herein and is not hydrogen and Q¹⁴ⁿ is a 5-6-membered saturated or partially unsaturated heterocyclic group containing at least one nitrogen atom and optionally containing a further heteroatom selected from N and O wherein Q¹⁴ⁿ is linked to C₁₋₆alkyl via a nitrogen atom or a carbon atom and wherein Q¹⁴, optionally bears 1, 2 or 3 substituents selected from C₂₋₅alkenyl, C₂₋₅alkynyl, C₁₋₆fluoroalkyl, C₁₋₆alkanoyl, aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, C₁₋₆fluoroalkanoyl, carbamoyl, C₁₋₄alkylcarbamoyl, di(C₁₋₄alkyl)carbamoyl, carbamoylC₁₋₆alkyl, C₁₋₄alkylcarbamoylC₁₋₆alkyl, di(C₁₋₄alkyl)carbamoylC₁₋₆alkyl, C₁₋₆alkylsulphonyl, C₁₋₆fluoroalkylsulphonyl, oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄alkyl, C₁₋₄alkylsulphonylC₁₋₄alkyl C₁₋₄alkoxycarbonyl, C₁₋₄aminoalkyl, C₁₋₄alkylamino, di(C₁₋₄aikyl)amino, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₄-alkoxy and a group —(—O—)_{f}(C₁₋₄alkyl)_{g} ring D (wherein f is 0 or 1, g is 0 or 1 and ring D is a 5-6-membered saturated or partially unsaturated heterocyclic group with 1-2 heteroatoms, selected independently from O, S and N, which heterocyclic group may bear one or more substituents selected from C₁₋₄alkyl)
or Q¹⁴ⁿ bears a single substitlent selected from methylenedioxy and ethylenedioxy);
(ii) Q¹⁵W³—
wherein W³ represents —NQ¹⁶C(O)—, —C(O)NQ¹⁷-, —S₂NQ¹⁸-, —NQ¹⁹SO₂— or —NQ²⁰- (wherein Q¹⁶, Q¹⁷, Q¹⁸, Q¹⁹ and Q²⁰ each independently represents C₂₋₅allenyl, C₂₋₅salkwyl, C₁₋₄haloalkyl), and Q¹⁵ is C₁₋₆haloallyl, C₂₋₅alkenyl or C₂₋₅alkynyl; and
(iii) Q²¹W⁴C_{1-alkylX}¹ wherein X¹ is as defined herein, W represents NQ²²C(O)—, C(O)NQ²³-, —SO₂NQ²⁴-, —NQ²⁵SO₂— or NQ²⁶- (wherein Q²², Q²³, Q²⁴, Q²⁵ and Q²⁶ each independently represents hydrogen, C₁₋₃alkyl, C₁₋₃allkoxYC₂₋₃alkyl, C₂₋₅alkenyl, C₂₋₅alkynyl or C₁₋₄haloalkyl), and Q²¹ represents C₁₋₆haloalkyl, C₂₋₅alkenyl or C₂₋₅alkynyl;
or a salt thereof.

2. A compound according to claim 1 wherein Z is —NH—.

3. A compound according to claim 1 or claim 2 wherein R³ is methoxy.

4. A compound according to any one of claims 1, 2 and 3 wherein X¹ is —O—.

5. A compound according to any one of the preceding claims wherein R² is selected from group (ii) of the groups (i), (ii) and (iii) defined in claim 1.

6. A compound according to any one of the preceding claims wherein R² is selected from group. (iii) of the groups (i), (ii) and (iii) defined in claim 1.

7. A compound according to any one of the preceding claims wherein R7 is selected from group (i) of the groups (i), (ii) and (iii) defmied in claim 1.

8. A compound according to claim 7 wherein R² is Q¹X¹- wherein X¹ is as defined in claim 1 and Q¹ is selected from one of the following ten groups:
1) Q, (wherein Q² is a 5-6-membered saturated or partially unsaturated heterocyclic group with 1-2 heteroatoms, selected independently from O, S and N, which heterocyclic group bears at least one substituent selected from C₂₋₅alkenyl, C₂₋₅alkynyl, aminoC₂₋₆alkanoyl, C₁₋₄alkylaminoC₂₋₆alkanoyl, di(C₁₋₄alkyl)aminoC₂₋₅alkanoyl, C₁₋₄alkoxyC₁₋₄alkylaminoC₂₋₆akanoyl, C₁₋₆fluoroalkanoyl, carbamoylC₁₋₆alkyl, C₁₋₄alkylcarbamoylC₁₋₆alkyl, di(C₁₋₄alkyl)carbamoylC₁₋₆alkyl, C₁₋₆alkylsulphonyl and C₁₋₄uoroalkylsulphonyl and which heterocyclic group may optionally bear a further 1 or 2 substituents selected from C₂₋₅alkenyl, C₂₋₅alkynyl, C₁₋₆fluoroalkyl, C₁₋₆alkanoyl, aminoC₂₋₆alkoyl, C₁₋₄alkyaminoC₂₋₆alkanoyl, di(C₁₋₄alkyl)aminoC₂₋₆alkanoyl, C₁₋₄alkanoyl, C₁₋₄fluoroalkamoyl, carbamoyl, C₁₋₄alkylcarbamoyl, di(C₁₋₄alkyl)carbamoyl, carbamoylC₁₋₆alkyl, C₁₋₄alkylcarbamoylC₁₋₆alkyl, di(Ci₁₋₄alkyl)carbamoylC₁₋₆alkyl, C₁₋₆alkylsulphonyl, C₁₋₆fluorosulphonyl, oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄alkyl, C₁₋₄alkylsulphonylC₁₋₄alkyl, C₁₋₄alkoxycarbonyl, C₁₋₄aminoalkyl, C₁₋₄alkylamino, di(C₁₋₄alkyl)amino, C₁₋₄alkylaminoC₁₋₄alkyl, di(C₁₋₄alky;)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g}ringD (wherein f is 0 or 1, g is 0 or 1 and ring D is a 5-6-membered saturated or partially unsaturated heterocyclic group with 1-2 heteroatoms, selected independently from O, S and N, which cyclic group may bear one or more substituents selected from C₁₋₄alkyl),
or Q² bears a single substituent selected fiom methylenedioxy and ethylenedioxy);
with the proviso that if Q¹ is Q² and X¹ is —O— then Q²must bear at least one substituent selected from C₂₋₅alkenyl, C₂₋₅alkynyl, C₁₋₄alkoxyC₁₋₄alkylaminoC₂-6alkanoyl, carbamoylC₁₋₆alkyl, C₁₋₄alkylcarbamoylC₁₋₄alkyl, and di(C₁₋₄alkyl)carbamoylC₁₋₆alkyl and optionally may bear a further 1 or 2 substituents as defined herein;
2) C₁₋₅alkylW¹Q² (wherein W¹ represents —O—, —S—, —SO—, —SO₂—, —C(O)—, —OC(O)—, —NQ³C(O)—, —C(O)NQ⁴, —SO₂NQ⁵, —NQ⁶SO₂— or NQ⁷- (wherein Q³ Q⁴, Q⁵, Q⁶ and Q⁷ each independently represents hydrogen, C₁₋₃alkyl, C₁₋₃alkoxyC₂₋₃alkyl, C₂₋₅alkenyl, C₂₋₅alkynyl or C₁₋₄haloalkyl) and Q² is as defined herein;
3) C₁₋₅alkylQ² (wherein Q² is as defined herein);
4) C₂₋₅alkenylQ² (wherein Q² is as defined herein);
5) C₂₋₅alkynyl 2 (wherein Q² is as defined herein);
6) C₁₋₄alkylW²C₁₋₄alkylQ² (wherein W² represents —O—, —S—, —SO—, —SO₂—, —C(O)—, —OC(O)—NQ⁸C(O)—, —C(O)NQ⁹-, —SO₂NQ¹⁰, —NQ¹SO₂— or —NQ²- (wherein Q⁸, Q⁹, Q¹⁰, Q¹¹ and Q¹² each independently represents hydrogen C₁₋₃alkyl, C₁₋₃alkoxyC₂₋₃alkyl, C₂₋₅alkenyl, C₂₋₅alkynyl or C₁₋₄haloalkyl) and Q² is as defined herein);
7) C₂₋₅alkenylW²C₁₋₄alkylQ² (wherein W² and Q² are as defined herein);
8) C₂₋₅alkylW²C₁₋₄alkylQ² (wherein W² and Q² are as defined herein);
9) C₁₋₄alkylQ1³(C₁₋₄alkyl)ⱼ(W²)ₖQ¹⁴ (wherein W² is as defined herein, j is 0 or 1, k is 0 or 1, and Q¹³ and Q¹⁴ are each independently selected from hydrogen, C₁₋₃alcyl, cyclopentyl, cyclohexyl and a 5-6-membered saturated or partially unsaturated heterocyclic group with 1-2 heteroatoms, selected independently from O, S and N, which C₁₋₃alkyl group may bear 1 or 2 substituents selected from oxo, hydroxy, halogeno and C₁₋₄alkoxy and which cyclic group may bear 1, 2 or 3 substituents selected from C₂₋₅alkenyl, C₂₋₅alkynyl, C₁₋₆fluoroalkyl, C₁₋₆alkanoyl, aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄akylaminoC₂₋₆alkanoyl, C₁₋₆fluoroalkanoyl, carbamoyl, C₁₋₄alkylcarbamoyl, di(C₁₋₄alkyl)carbamoyl, carbarmoylC₁₋₆alkyl, C₁₋₄alkylcarbamoylC₁₋₄alkyl, di(C₁₋₄alkyl)carbamoylC₁₋₆alkyl, C₁₋₆alkylsulphonyl, C₁₋₆fluoroalkylsulphonyl, oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄alkyl, C₁₋₄alkylsulphonylC₁₋₄alkyl, C₁₋₄alkoxycarbonyl, C₁₋₄aminoalkyl, C₁₋₄alkylamino, di(C₁₋₄alkyl)amino, C₁₋₄alkylaminoC₁₋₄alkyl, di(C₁₋₄alkyl)amioC₁₋₄alkyl, C₁₋₄alkylamioC₁₋₄alkoxy, di(C₁₋₄alkyl)aminoC₁₋₄alkoxy and a group —(—O—)_{f}(C₁₋₄alkyl)_{g} ring D (wherein f is 0 or 1, g is 0 or 1 and ring D is a 5-6-membered saturated or partially unsaturated heterocyclic group with 1-2 heteroatoms, selected independently from O, S and N, which heterocyclic group may bear one or more substituents selected from C₁₋₄alkyl), with the provisos that Q¹³ cannot be hydrogen and one or both of Q¹³ and Q¹⁴ must be a 5-6-membered saturated or partially unsaturated heterocyclic group as defined herein which heterocyclic group bears at least one substituent selected from C₂₋₅alkenyl, C₂₋₅akyl, C₁₋₆alkanoyl, aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, C₁₋₆fluoroalkanoyl, carbamoyl, C₁₋₄alkylcarbamoyl, di(C₁₋₄alkyl)carbamoyl, carbamoylC₁₋₆alkyl, C₁₋₄alkylcarbamoylC₁₋₆alkyl, di(C₁₋₄alkyl)carbamoylC₁₋₆alkyl, C₁₋₆alkylsulphonyl and C₁₋₆fluoroalkylsulphonyl and which heterocyclic group optionally bears 1 or 2 further substituents selected from those defined herein); and
10) C₁₋₄alkylQ¹³-C(O)-C₁₋₄alkylQ¹⁴ⁿ wherein Q¹³ is as defined herein and is not hydrogen and Q¹⁴ⁿ is a 5-6-membered saturated or partially unsaturated heterocyclic group containing at least one nitrogen atom and optionally containing a further heteroatom selected from N and O wherein Q¹⁴ⁿ is linked to C₁₋₆alkyl via a nitrogen atom and wherein Q¹⁴ⁿ optionally bears 1, 2 or 3 substituents selected from C₂₋₅alkenyl, C₂₋₅alkynyl, C₁₋₆fluoroalkyl, C₁₋₆alkanoyl, aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, C₁₋₆fluoroalkanoyl, carbamoyl, C₁₋₄alkylcarbamoyl, di(C₁₋₄alkyl)carbamoyl, carbamoylC₁₋₆alkyl, C₁₋₄alkylcarbamoylC₁₋₆alkyl, di(C₁₋₄alkyl)carbamoylC₁₋₆alkyl, C₁₋₆alkylsulphonyl, C₁₋₆fluoroalkylsulphonyl, oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄alkyl, C₁₋₄alkylsulphonylC₁₋₄alkyl, C₁₋₄alkoxycarbonyl, C₁₋₄aminoalkyl, C₁₋₄alkylamino, di(C₁₋₄alkyl)amino, C₁₋₄alkylaminoC₁₋₄alkyl, di(C₁₋₄alkyl)amiioC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g} ring D (wherein f is 0 or 1, g is 0 or 1 and ring D is a 5-6-membered saturated or partially unsaturated heterocyclic group with 1-2 heteroatoms, selected independently from O, S and N, which heterocyclic group may bear one or more substituents selected from C₁₋₄alkyl) or Q¹⁴ⁿ bears a single substituent selected from methylenedioxy and ethylenedioxy).

9. A compound according to claim 7 wherein R² is Q¹X¹- wherein X¹ is as defined in claim 1 and Q¹ is selected from one of the following ten groups:
1) Q² (wherein Q² is a 5-6-membered saturated or partially unsaated heterocyclic group with 1-2 heteroatoms, selected independently from O, S and N, which heterocyclic group bears at least one substituent selected from aminoC₂₋₆alkanoyl, C₁₋₄alkylaminoC₂₋₆alkanoyl, di(C₁₋₄alkyl)aminoC₂₋₆akanoyl, C₁₋₄alkoxyC₁₋₄alkylaminoC₂₋₆alkanoyl, carbamoylC₁₋₆alkyl, C₁₋₄alkylcarbamoylC₁₋₆alkyl and di(C₁₋₄alkyl)carbamoylC₁₋₆-alkyl and which heterocyclic group may optionally bear a further 1 or 2 substituents selected from C₂₋₅alkenyl, C₂₋₅alkynyl, C₁₋₆fluoroalkyl, C₁₋₆alkanoyl, aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, C₁₋₄fluoroalkanoyl, carbamoyl, C₁₋₄alkylcarbamoyl, di(C₁₋₄alkyl)carbamoyl, carbamoylC₁₋₆alkyl, C₁₋₄alkylcarbamoylC₁₋₆alkyl, di(C₁₋₄alkyl)carbamoylC₁₋₆alkyl, C₁₋₆alkylsulphonyl, C₁₋₆fluoroalkylsulphonyl, oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄alkyl, C₁₋₄alkylsulphonylC₁₋₄alkyl, C₁₋₄alkoxycabonyl, C₁₋₄aminoalkyl, C₁₋₄alkylamio, di(C₁₋₄alkyl)amino, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g} ring D (wherein f is 0 or 1, g is 0 or 1 and ring D is a 5-6-membered saturated or partially unsatated heterocyclic group with 1-2 heteroatoms, selected independently from O, S and N, which cyclic group may bear one or more substituents selected from C₁₋₄alkyl),
or Q² bears a single substituent selected from methylenedioxy and ethylenedioxy);
with the proviso that if Q¹ is Q² and X¹ is —O— then Q² must bear at least one substituent selected from C₁₋₄alkoxyC₁₋₄alkylaminoC₂₋₆alkanoyl, carbamoylC₁₋₆alkyl, C₁₋₄alkylcarbamoylC₁₋₆alkyl, and di(C₁₋₄alkyl)carbamoylC₁₋₆alkyl and optionally may bear a further 1 or 2 substituents as defined herein;
2) C₁₋₅alkylW¹Q² (wherein W¹ represents —O—, —S—, —SO—, —SO₂—, —C(O)—, —OC(O)—, —NQ³C(O)—, —C(O)NQ⁴-, —SO₂NQ⁵-, —NQ⁶SO₂— or —NQ⁷- (wherein Q³, Q⁴, Q⁵, Q⁶ and Q⁷ each independently represents hydrogen, C₁₋₃alkyl, C₁₋₃alkoxyC₂-₃alkyl, C₂₋₅alkenyl, C₂₋₅alkynyl or C₁₋₄haloalkyl) and Q² is as defined herein;
3) C₁₋₅alkylQ² (wherein Q² is as defined herein);
4) C₂₋₅alkenylQ² (wherein Q² is as defined herein);
5) C₂₋₅alkynylQ² (wherein Q² is as defined herein);
6) C₁₋₄alkylW²C₁₋₄alkylQ² (wherein W² represents —O—, —S—, —SO—, —SO₂—, —C(O)—, —OC(O)—NQ⁸C(O)—, —C(O)NQ⁹-, —SO₂NQ¹⁰, —NQ¹¹SO₂— or —NQ¹²- (wherein Q⁸, Q⁹, Q¹⁰, Q¹¹ and Q¹² each independently represents hydrogen, C₁₋₃alkyl, C₁₋₃alkoxyC₂₋₃alkyl, C₂₋₅alkenyl, C₂₋₅alkynyl or C₁₋₄haloalkyl) and Q² is as defined herein);
7) C₂₋₅alkenylW²C₁₋₄alkylQ² (wherein W² and Q² are as defined herein);
8) C₂₋₅alkynylW²C₁₋₄alkylQ² (wherein W² and Q² are as defined herein);
9) C₁₋₄alkylQ¹³(C₁₋₄alkyl)ⱼ(W²)ₖQ¹⁴ (wherein W² is as defined herein, j is 0 or 1, k is 0 or 1, and Q¹³ and Q¹⁴ are each independently a 5-6-membered saturated or partially unsaturated heterocyclic group with 1-2 heteroatoms, selected independently from O, S and N, which heterocyclic group may bear 1, 2 or 3 substituents selected from C₂₋₅alkenyl, C₂₋₅alkynyl, C₁₋₆fluoroalkyl, C₁₋₆alkanoyl, aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, C₁₋₆fluoroalkanoyl, carbamoyl, C₁₋₄alkylcarbamoyl, di(C₁₋₄alkyl)carbamoyl, carbamoylC₁₋₆alkyl, C₁₋₄alkycarbamoylC₁₋₆alkyl, di(C₁₋₄alkyl)carbamoylC₁₋₆alkyl, C₁₋₆alkylsulphonyl, C₁₋₆fluoroalkylsulphonyl, oxo, hydroxy, halogeno, cyano, C₁₋₄cyanoalkyl, C₁₋₄alkyl, C₁₋₄hydroxyalkyl; C₁₋₄alkoxy, C₁₋₄alkoxyC₁₋₄alkyl, C₁₋₄alkylsulphonylC₁₋₄alkyl, C₁₋₄alkoxycarbonyl, C₁₋₄aminoalkyl, C₁₋₄alkylamino, di(C₁₋₄alkyl)amino, C₁₋₄alkylaminoC₁₋₄alkyl, di(C₁₋₄alkyl)aminoC₁₋₄alkyl, C₁₋₄alkylaminoCl₁₋₄alkoxy, di(C₁₋₄alkyl)aminoC₁₋₄alkoxy and a group —(—O—)_{f}(C₁₋₄alkyl)_{g} ring D (wherein f is 0 or 1, g is 0 or 1 and ring D is a 5-6-membered saturated or partially-unsaturated heterocyclic group with 1-2 heteroatoms, selected independently from O, S and N, which heterocyclic group may bear one or more substituents selected from C₁₋₄alkyl), with the proviso that one or both of Q¹³ and Q¹⁴ bears at least one substituent selected from aminoC₂₋₆alkanoyl, C₁₋₄alkylaminoC₂₋₆alkanoyl, di(C₁₋₄aminoC₂₋₆alkanoyl, C₁₋₄alkoxyC₁₋₄alkylaminoC₂₋₆alkanoyl, carbamoylC₁₋₆alkyl, C₁₋₄alkylcarbamoylC₁₋₆alkyl and di(C₁₋₄alky)carbamoylC₁₋₆alkyl, and which heterocyclic group optionally bears 1 or 2 fuirther substituents selected from those defined herein); and
10) C₁₋₄alylQ¹³-C(O)-C₁₋₄alkylQ¹⁴ⁿ wherein Q¹³ is as defined herein and Q¹⁴ⁿ is a 5-6-membered saturated or partially unsaturated heterocyclic group containing at least one nitrogen atom and optionally containing a further heteroatom selected from N and O wherein Q¹⁴ⁿ is linked to C₁₋₆alkyl via a nitrogen atom or a carbon atom and wherein Q¹⁴ⁿ optionally bears 1, 2 or 3 substituents selected from C₂₋₅alkenyl, C₂₋₅alkynyl, C₁₋₆fluoroalkyl, C₁₋₆alkanoyl, aminoC₂₋₆alkanoyl, C₁₋₄alkyaminoC₂₋₆alkanoyl, di(C₁₋₄alkyl)aminoC₂₋₆alkanoyl, C₁₋₄alkoxyC₁₋₄alkylaminoC₂₋₆alkanoyl, C₁₋₆fluoroalkanoyl, carbamoyl, C₁₋₄alkylcarbamoyl, di(C₁₋₄alkyl)carbamoyl, carbamoylC₁₋₆alkyl, C₁₋₄alkylcarbamoylC₁₋₆-alkyl, di(C₁₋₄alkyl)carbamoylC₁₋₆alkyl, C₁₋₆alkylsulphonyl, C₁₋₆fluoroalkylsulphonyl, oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄alkyl, C₁₋₄alkylsulphonylC₁₋₄alkyl, C₁₋₄alkoxycarbonyl, C₁₋₄aminoalkyl, C₁₋₄alkylamino, di(C₁₋₄alkyl)amino, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₋₄alkoxy and a group —(O—)_{f}(C₁₋₄allkyl)_{g}ringD (wherein f is 0 or 1, g is 0 or 1 and ring D is a 5-6membered saturated or partially unsaturated heterocyclic group with 1-2 heteroatoms, selected independently from O, S and N, which heterocyclic group may bear one or more substituents selected from C₁₋₄alkyl) or Q¹⁴ⁿ bears a single substituent selected from methylenedioxy and ethylenedioxy).

10. A compound according to claim I of the formula Ia: 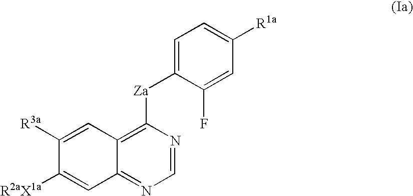
wherein:
Za is —NH—, —O— or —S—;
R^{1a} represents bromo or chloro;
R^{3a} represents C₁₋₃alkoxy or hydrogen;
X^{1a} represents —O—, —S— or —NR^{4a}— wherein R^{4a} is hydrogen, C₁₋₃alkyi or C₁₋₃alkoxyC₂₋₃alkyl;
R^{2a} is selected from one of the following groups:
1) C₁₋₅alkylR^{5a} (wherein R^{5a} is a 5- or 6-membered heterocyclic ring selected from morpholine, pyrrolidine, piperidine and piperazine which heterocyclic ring bears at least one substituent selected from aminoC₂₋₄alkanoyl, C₁₋₄alkylaminoC₂₋₄alkanoyl, di(C₁₋₄alkyl)aminoC₂₋₄alkanoyi, C₁₋₄alkoxyC₁₋₄alkylaminoC₂₋₄alkanoyl, methylenedioxy and ethylenedioxy);
2) C₂₋₅alkenylR^{5a} (wherein R^{5a} is as defined herein);
3) C₂₋₅alkynylR^{5a} (wherein R^{5a} is as defined herein);
4) C₁₋₅alkylR^{6a} C(O)(CH₂)ₘₐR^{7a} (wherein ma is 1 or 2, R⁶a is a 5- or 6-membered heterocyclic ring selected from morpholine, pyrrolidine, piperidine and piperazine which heterocyclic ring may bear one or two substituents selected from fluoro, hydroxy and methyl, and R^{7a} is a 5- or 6-membered heterocyclic ring selected from pyrrolidine, piperidie, piperazine and morpholine which heterocyclic ring is linked to (CH₂)ₘₐ via a nitrogen atom or a carbon atom and which heterocyclic ring may bear one or more substituents selected from hydroxy, halogeno, C₁₋₄alkanoyl, methylenedioxy and ethylenedioxy); and
5) C₁₋₅alkylR^{6a}(CH₂)ₘₐC(O)R^{8a} (wherein ma and R⁶a are as defined herein and R^{8a} is a 5- or 6-membered heterocyclic ring selected from pyrrolidine, piperidine, piperazine and morpholine which heterocyclic ring is linked to C(O) via a nitrogen atom or a carbon atom and which heterocyclic ring may bear one or more substituents selected from hydroxy, halogeno, C₁₋₄alkanoyl, methiylenedioxy and ethylenedioxy)
or a salt thereof.

11. A compound according to claim I of the formula Ib: 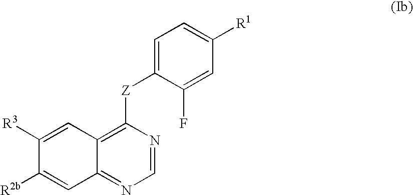
wherein:
Z, R¹ and R³ are as defined in claim 1 and
R^{2b} is selected from one of the following three groups:
(i) Q^{1b}X¹-
wherein X¹ is as defined in claim 1 and Q^{1b} is selected from one of the following ten groups:
1) Q^{2b} (wherein Q^{2b} is a 5-6-membered sated or partially unsaturated heterocyclic group with 1-2 heteroatoms, selected independently from O, S and N, which heterocyclic group bears at least one substituent selected from C₂₋₅alkenyl, C₂₋₅alkyl, C₁₋₆fluoroalkyl, aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, C₁₋₆fluoroalkanoyl, carbamoylC₁₋₆alkyl, C₁₋₄alkylcarbamoylC₁₋₆alkyl, di(C₁₋₄alkyl)carbamoylC₁₋₆alkyl and C₁₋₄fluoroalkylsulphonyl and which heterocyclic group may optionally bear a further 1 or 2 substituents selected from C₂₋₅alkenyl, C₂₋₅alkynyl, C₁₋₆fluoroalkyl, C₁₋₆alkanoyl, aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, C₁₋₆fluoroalkanoyl, carbamoyl, C₁₋₄alkylcarbamoyl, di(C₁₋₄alkyl)carbamoyl, carbamoylC₁₋₆alkyl, C₁₋₄alkylcarbamoylC₁₋₆alkyl, di(C₁₋₄alkyl)carbamoylC₁₋₆alkyl, C₁₋₆alkylsulphonyl, C₁₋₆fluoroalkylsulphonyl, oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄alkyl, C₁₋₄alkylsulphonylC₁₋₄alkyl, C₁₋₄alkoxycarbonyl, C₁₋₄aminoalkyl, C₁₋₄alkylamino, di(C₁₋₄alkyl)amino, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g} ring D (wherein f is 0 or 1, g is 0 or 1 and ring D is a 5-6-membered saturated or partially unsaturated heterocyclic group with 1-2 heteroatoms, selected independently from O, S and N, which cyclic group may bear one or more substituents selected from C₁₋₄alkyl),
or Q^{2b} bears a single substituent selected from methylenedioxy and ethylenedioxy);
with the proviso that if Q^{1b} is Q^{2b} and X¹ is —O— then Q^{2b} must bear at least one substituent selected from C₂₋₅alkenyl, C₂₋₅alkynyl, C₁₋₄alkoxyC₁₋₄alkylaminoC₂₋₄alkanoyl, carbamoylC₁₋₆alkyl, C₁₋₄alkylcarbamoylC₁₋₆alkyl, and di(C₁₋₄alkyl)carbamoylC₁₋₆alkyl and optionally may bear a further 1 or 2 substituents as defined herein;
2) C₁₋₅alkylW¹Q² (wherein W¹ and Q² are as defined in claim 1);
3) C₁₋₅alkylQ^{2b} (wherein Q^{2b} is as defined herein);
4) C₂₋₅alkenylQ² (wherein Q² is as defined in claim 1);
5) C₂₋₅alkynylQ² (wherein Q² is as defined in claim 1);
6) C₁₋₄alkylW²C₁₋₄alkylQ² (wherein W² and Q² are as defined in claim 1);
7) C₂₋₅alkenylW²C₁₋₄alkylQ² (wherein W² and Q² are as defined in claim 1);
8) C₂₋₅alkynylW²C₁₋₄-alkylQ² (wherein W² and Q² are as defined in claim 1);
9) C₁₋₄alkylQ^{13b}(C₁₋₄alkyl)ⱼ(W²)ₖQ^{14b} (wherein W² is as defined in claim 1, j is 0 or 1, k is 0 or 1, and Q^{13b} and Q^{14b} are each independently selected from hydrogen, C₁₋₃alkyl, cyclopentyl, cyclohexyl and a 5-6-membered saturated or partially unsaturated heterocyclic group with 1-2 heteroatoms, selected independently from O, S and N, which C₁₋₃alkyl group may bear 1 or 2 substituents selected from oxo, hydroxy, halogeno and C₁₋₄alkoxy and which cyclic group may bear 1, 2 or 3 substituents selected from C₂₋₅alkenyl, C₂₋₅alkynyl, C₁₋₆fluoroalkyl, C₁₋₆alkanoyl, aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, C₁₋₆fluoroalkanoyl, carbamoyl, C₁₋₄alkylcarbamoyl, di(C₁₋₄alkyl)carbamoyl, carbamoylC₁₋₆alkyl, C₁₋₄alkylcarbamoylC₁₋₆alkyl, di(C₁₋₄alkyl)carbamoylC₁₋₆alkyl, C₁₋₆alkylsulphonyl, C₁₋₆fluoroalkylsulphonyl, oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄alkyl, C₁₋₄alkylsulphonylC₁₋₄alkyl, C₁₋₄alkoxycarbonyl, C₁₋₄aminoalkyl, C₁₋₄alkylamino, di(C₁₋₄alkyl)amino, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₋₄alkoxy and a grroup —(O—)_{f}(C₁₋₄alkyl)_{g} ring D (wherein f is 0 or 1, g is 0 or 1 and ring D is a 5-6-membered saturated or partially unsaturated heterocyclic group with 1-2 heteroatoms, selected independently from O, S and N, which heterocyclic group may bear one or more substituents selected from C₁₋₄alkyl), with the provisos that Q^{13b} cannot be hydrogen and one or both of Q^{13b} and Q^{14b} must be a 5-6-membered saturated or partially unsaturated heterocyclic group as defined herein which heterocyclic group bears at least one substituent selected from C₂₋₅alkenyl, C₂₋₅alkynyl, C₁₋₆fluoroalkyl, aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylamioC₂₋₆alkanoyl, C₁₋₆fluoroalkanoyl, carbamoyl, C₁₋₄alkylcarbamoyl, di(C₁₋₄alkyl)carbamoyl, carbamoylC₁₋₆alkyl, C₁₋₄alkycarbamoylC₁₋₄alkyl, di(C₁₋₄alkyl)carbamoylC₁₋₆alkyl and C₁₋₆fluoroalkylsulphonyl and which heterocyclic group optionally bears 1 or 2 further substituents selected from those defined herein); and
10) C₁₋₄alkylQ¹³-C(O)-C₁₋₄alkylQ¹⁴ⁿ (wherein Q¹³ and Q¹⁴ⁿ are as defined in claim 1);
(i) Q¹⁵W³- (wherein W³ and Q¹⁵ are defined in claim 1); and
(iii) Q²¹W⁴C₁₋₅alkylX¹ (wherein X¹, W⁴ and Q²¹ are as defined in claim 1);
or a salt thereof.

12. A compound according to claim 11 wherein R^{2b} is Q^{1b}X¹-
wherein X¹ is as defined in claim 1 and Q^{1b} is selected from one of the following ten groups:
1) Q^{2b} (wherein (b is a 5-6membered saturated or partially unsaturated heterocyclic group with 1-2 heteroatoms, selected independeently from O, S and N, which heterocyclic group bears at least one substituent selected from C₁₋₄alkoxyC₁₋₄alkylamioC₂₋₆alkanoyl, C₁₋₄alkylcarbamoylC₁₋₆alkyl and di(C₁₋₄alkyl)carbamoylC₁₋₆alkyl and which heterocyclic group may optionally bear a further 1 or 2 substituents selected from C₂₋₅alkenyl, C₂₋₅alkynyl, C₁₋₆fluoroalkyl, C₁₋₆alkanoyl, aminoC₂₋₆alkanoyl, C₁₋₄alkylaminoC₂₋₆alkanoyl, di(C₁₋₄alkyl)aminoC₂₋₆alkanoyl, C₁₋₄alkoxyC₁₋₄alkylaminoC₂₋₆alkanoyl, C₁₋₆fluoroalkanoyl, carbamoyl, C₁₋₄alkylcarbamoyl, di(C₁₋₄alkyl)carbamoyl, carbamoylG₁₋₆alkyl, C₁₋₄alkylcarbamnoylC₁₋₆alkyl, di(C₁₋₄alkylcarbamoylC₁₋₆alkyl, C₁₋₆alkylsulphonyl, C₁₋₆fluoroalkylsulphonyl, oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄alkyl, C₁₋₄alkysulphonylC₁₋₄alkyl, C₁₋₄alkoxycarbonyl, C₁₋₄aminoalkyl, C₁₋₄alkylamino, di(C₁₋₄alkyl)amino,, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g} ring D (wherein f is 0 or 1, g is 0 or 1 and ring D is a 5-6-membered saturated or partially unsaturated heterocyclic group with 1-2 heteroatoms, selected independently from O, S and N, which cyclic group may bear one or more substituents selected from C₁₋₄alkyl),
or Q^{2b} bears a single substituent selected from methylenediioxy and ethylenedioxy);
2) C₁₋₅alkylW¹Q^{2b} (wherein W¹ is as defined in claim 1 and Q^{2b} is as defined herein);
3) C₁₋₅alkylQ^{2b} (wherein Q^{2b} is as deffned herein);
4) C₂₋₅alkenylQ^{2b} (wherein Q^{2b} is as defined herein);
5) C₂₋₅alkynylQ^{2b} (wherein Q^{2b} is as defined herein);
6) C₁₋₄alkylW²C₁₋₄alkylQ^{2b} (wherein W² is as defined in claim 1 and Q^{2b} is as defined herein);
7) C₂₋₅alkenylW²C₁₋₄alkylQ^{2b} (wherein W² is as defined in claim 1 and Q^{2b} is as defined herein);
8) C₂₋₅alkynlW²C₁₋₄alkylQ^{2b} (wherein W² is as defied in claim 1 and Q^{2b} is as defined herein);
9) C₁₋₄alkylQ^{13b}(C₁₋₄alkyl)ⱼ(W²)ₖQ^{14b} (wherein W² is as defined in claim 1, j is 0 or 1, k is 0 or 1, and Q^{13b} and Q^{14b} are each independently selected from hydrogen, C₁₋₃alkyl, cyclopentyl, cyclohexyl and a 5-6-membered saturated or partially umnasurated heterocyclic group with 1-2 heteroatoms, selected independently from O, S and N, which C₁₋₃alkyl group may bear 1 or 2 substituents selected from oxo, hydroxy, halogeno and C₁₋₄alkoxy and which cyclic group may bear 1, 2 or 3 substituents selected from C₂₋₅alkenyl, C₂₋₅alkynyl, C₁₋₆fluoroalkyl C₁₋₆alkanoyl, aminoC₂₋₆alkanoyl, C₁₋₄akylaminoC₂₋₆akanoyl, di(C₁₋₄alkyl)aminoC₂₋₆alkanoyl, C₁₋₄alkoxyC₁₋₄alkylaminoC₂₋₆alkanoyl, C₁₋₆fluoroalkanoyl, carbamoyl, C₁₋₄alkylcarbaraoyl, di(C₁₋₄alkyl)carbamoyl, carbamoylC₁₋₆alkyl, C₁₋₄alkylcarbamoylC₁₋₆alkyl, di(C₁₋₄alkyl)carbamoylC₁₋₆alkyl, C₁₋₆-alkylsulphonyl, C₁₋₆fluoroalkylsulphonyl, oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄alkyl, C₁₋₄alkylsulphonylC₁₋₄alkyl, C₁₋₄alkoxycarbonyl, C₁₋₄amminoalkyl, C₁₋₄alkylamino, di(C₁₋₄alkyl)amino, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g}ringD (wherein f is 0 or 1, g is 0 or 1 and ring D is a 5-6-membered saturated or partially unsaturated heterocyclic group with 1-2 heteroatoms, selected independently from O, S and N, which heterocyclic group may bear one or more substituents selected from C₁₋₄alkyl), with the provisos that Q^{13b} cannot be hydrogen and one or both of Q^{13b} and Q^{14b} must be a 5-6-membered saturated or partially unsaturated heterocyclic group as defined herein which heterocyclic group bears at least one substituent selected from C₁₋₄alkoxyC₁₋₄alkylaminoC₂₋₆alkanoyl, C₁₋₄alkylcarbamoylC₁₋₆alkyl and di(C₁₋₄alkyl)carbamoylC₁₋₆alkyl and which heterocyclic group optionally bears 1 or 2 further substituents selected from those defined herein); and
10) C₁₋₄alkylQ^{13b}-C(O)-C₁₋₄alklQ^{14b} (wherein Q^{13b} and Q^{14b} are as defined herein and with the provisos that Q^{13b} cannot be hydrogen and one or both of Q^{13b} and Q^{14b} must be a 5-6-membered saturated or partially unsaturated heterocyclic group as defined herein which heterocyclic group bears at least one substituent selected from C₁₋₄alkoxyC₁₋₄alkylaminoC₂₋₆alkanoyl, C₁₋₄alkylcarbamoylC₁₋₆alkyl and di(C₁₋₄alkyl)carbamoylC₁₋₆alkyl and which heterocyclic group optionally bears 1 or 2 fuirther substituents selected from those defined herein).

13. A compound according to claim 1 selected from:
4(4bromo-2-fluoroanilino)-7-({1-[(N-N-dimethylamino)acetyl]piperidin-4-yl}methoxy)-6-methoxyquinazoline,
4-(4-chloro-2-fluoroanilino)-7-({1-[(N,N-diimethylamino)acetyl]piperidin-4ylmethoxy)-6-methoxyquinazoline,
4-(4-chloro-2-fluoroanilino)-6-methoxy-7-{[1-(pyrrolidin-1-ylacetyl)piperidin-4-yl]methoxy}quinazoline,
4-(4chloro-2-fluoroanilino)-6-methoxy-7-[1-(piperidin-1-ylacetyl)piperidin-4-yl]methoxy}quinazoline,
4-chloro-2-fluoroanilino)methoxy-7-{[1-morpholin-4-ylacetyl)piperidin-4-yl]methoxy}quinazoline,
4-(chloro-2-fluoroanilino)-methoxy-7-{1-[(3aR,6aS)-tetrahydro-5H-[1,3]dioxolo[4,5-c]pyrrol-5-ylacetyl]piperidin-4-yl}methoxy)quinazoline,
7-({1-[(4-acetylpiperazin-1-yl)acetyl]piperidine-4-yl}methoxy-4-chloro-2-fluoroanilino)-6-methoxyquinazoline,
(3S)-4-(4-chloro-2-fluoroanilio)-7-({1-[(3-hydroxypyrrolidin-1-yl)acetyl]piperidin-4-yl}methoxy)-6-methoxyquinazoline,
4-(4-chloro-2-fluoroanilino)-6-methoxy-7-[(1-{[N-(2-methoxyethyl)amino]acetyl}piperidin-4-yl)methoxy]quinazolie,
4-(4-chloro-2-fluoroanilino)-6-methoxy-7-({1-[(N-methylamio)acetyl]piperidin-4-yl}methoxy)quinazoline,
4-(4-chloro-2-fluoroanilino)-7-({1-[(3,3-difluoropyrrolidin-1-yl)acetyl]piperidin-4-yl}methoxy)-6-methoxyquinazoline,
4-(4-chloro-2-fluoroanilino)-7-(2-{1-[(N,N-dimethylamino)acetyl]piperidin-4-yl}ethoxy)-6-methoxyquinazoline,
4-(4-bromo-2-fluoroanilino)-7-(2-{1-[(N,N-dimethylamino)acetyl]piperidin-4-yl}ethoxy)-6-methoxyquinazoline,
4-(4-chloro-2-fluoroanilino)7-({(3R)-1-[(N,N-dimethylamino)acetyl]piperidin-3-yl}methoxy)-6-methoxyquinazoline,
4-(4-Chloro-2-fluoroanilino)-7-({(3S)-[(N,N-dimethylamino)acetyl]piperidin-3-yl}methoxy)6-methoxyquinazoline,
4-(4-bromo-2-fluoroanilino-6-methoxy-7-{3-[3aR,6aS)-tetrahydro-5H-[1,3)dioxolo[4,5-c]pyrrol-5-yl]propoxy}quinazoline,
4-(4-bromo-2-fluoroanilino)-6-methoxy-7-{2-[(3aR,6aS)-tetrahydro-5H-[1,3]dioxolo[4,5-c]pyrrol-5-yl]ethoxy}quinazoline,
and salts thereof.

14. A compound according to any one of the preceding claims in the form of a pharmaceutically acceptable salt.

15. A process for the preparation of a compound according to claim 1 of the formula I or salt thereof which comprises:
(a) the reaction of a compound of the formula II: 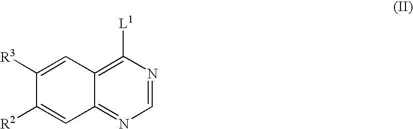
wherein R² and R³ are as defined in claim 1 and L¹ is a displaceable moiety, with a compound of the formula III: 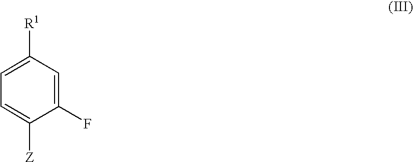
wherein R¹ and Z are as defined in claim 1;
(b) the reaction of a compound of the formula IV: 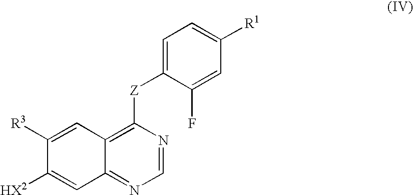
wherein Z, R¹ and R³ are as defined in claim 1 with a compound of formula V:
R⁵-L¹ (V)
wherein R⁵ is Q¹, Q¹⁵ or Q²¹W⁴C₁₋₅alkyl, X² is X¹ or W³ and L¹ is as defined herein and wherein Q¹, Q¹⁵, Q²¹, W⁴, X¹ and W³ are as defined in claim 1;
(c) the reaction of a compound of the formula VI: 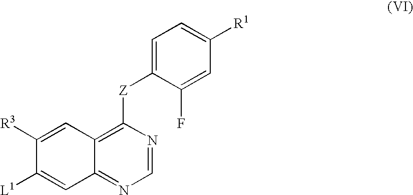
with a compound of the formula VIIa-c:
Q¹-X¹—H (VIIa)
Q¹⁵-W³—H (VIIb)
Q²¹-W⁴-C₁₋₅alkyl-X¹—H (VIIc)
(wherein L¹ is as defined herein and R¹, R³, Z, Q¹, Q¹⁵, Q²¹ W³, W⁴ and X¹ are as defined in claim 1);
(d) the deprotection of a compound of the formula VIII: 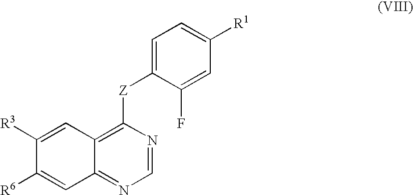
wherein R¹, R³ and Z are all as defined in claim 1, and R⁶ represents a protected R² group wherein R² is as defined in claim 1 but additionally bears one or more protecting groups P²;
(e) the addition of a substituent to a compound of the formula IX: 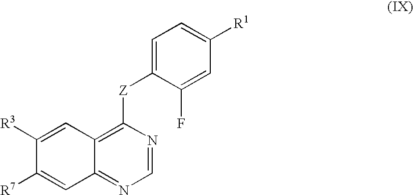
wherein R¹, R³ and Z are as defined in claim 1, and R⁷ represents an R² group which has yet to be substituted with its final substituent;
and when a salt of a compound of formula I is required, reaction of the compound obtained with an acid or base whereby to obtain the desired salt.

16. A pharmaceutical composition which comprises a compound ofthe formula I as defined in claim 1 or a pharmaceutically acceptable salt thereof, in association with a pharmaceutically acceptable excipient or carrier.

17. Use of a compound of the formula I as defined in claim 1 or a pharmaceutically acceptable salt thereof in the manufacture of a medicament for use in the production of an antiangiogenic and/or vascular permeability reducing effect in a warm-blooded animal.

18. A method for producing an antiangiogenic and/or vascular permeability reducing effect in a warm-blooded animal, such as a human being, in need of such treatment which comprises administering to said animal an effective amount of a compound of formula I as defined in claim 1 or a pharmaceutically acceptable salt thereof.
